(19) United States
(12) Patent Application Publication
(10) Pub. No.: US 2006/0073526 A1
(43) Pub. Date: Apr. 6, 2006

(54) Antibodies, assay method by using them and judgment method for pancreas cancer

(75) Inventors: Takatoshi Hamada, Hyogo (JP); Shinji Matsugi, Hyogo (JP); Takumi Tanaka, Hyogo (JP)

Correspondence Address: FOLEY AND LARDNER LLP;SUITE 500, 3000 K STREET NW, WASHINGTON, DC, 20007, US

(73) Assignee: WAKO PURE CHEMICAL INDUSTRIES, LTD

(21) Appl. No.: 10/957,746

(22) Filed: Oct. 5, 2004

(51) Int. Cl. G01N 33/574; C07K 16/40

(52) U.S. Cl. 435/7.23; 530/388.26

(57) ABSTRACT

An antibody which specifically binds to carboxypeptidase A1 (CPA1) and procarboxypeptidase A1 (PCPA1), an antibody which specifically binds to carboxypeptidase A2 (CPA2) and procarboxypeptidase A2 (PCPA2), an antibody which specifically binds to PCPA1, and an antibody which specifically binds to PCPA2, and a hybridoma which produces a monoclonal antibody thereof, are provided. The present invention relates to an antibody which specifically binds to CPA1 and PCPA1, an antibody which specifically binds to CPA2 and PCPA2, an antibody which specifically binds to PCPA1 and an antibody which specifically binds to PCPA2, and a hybridoma which produces a monoclonal antibody thereof; a method for immunologically assaying one or more amount selected from an amount of CPA1, an amount of CPA2, an amount of PCPA1, an amount of PCPA2, and a total amount of two or more amounts selected therefrom (an amount of an object to be assayed); a judging method for pancreas cancer; and a kit therefore. According to the present invention, amounts of the above-described objects to be assayed can be more simply, more quickly and more specifically assayed than in a conventional method. And based on the results assayed, it is possible to judge pancreas cancer or not, especially pancreas cancer at an early stage (stages 1, 2, and the like) or not.

### BACKGROUND OF THE INVENTION

The present invention relates to an antibody which specifically binds to carboxypeptidase A1 and procarboxypeptidase A1, an antibody which specifically binds to carboxypeptidase A2 and procarboxypeptidase A2, an antibody which specifically binds to procarboxypeptidase A1 and an antibody which specifically binds to procarboxypeptidase A2; a method for immunologically assaying one or more amounts selected from an amount of carboxypeptidase A1, an amount of carboxypeptidase A2, an amount of procarboxypeptidase A1, an amount of procarboxypeptidase A2 and a total amounts of two or more amounts selected therefrom, using one or more kinds thereof; and a judgment method for pancreas cancer.

The carboxypeptidase A (hereinafter designates as CPA) is an enzyme, which releases aromatic lipophilic amino acids sequentially from C-terminal in protein, and two types of isozymes of carboxypeptidase A1 (hereinafter designates as CPA1) and carboxypeptidase A2 (hereinafter designates as CPA2) are known (non-patent reference 1: Eur. J. Biochem. 211, 381-389 (1993)).

CPA1 and CPA2 are activated type CPA (CPA1 and CPA2) and are derived from each of proenzymes, i.e. procarboxypeptidase A1 (hereinafter designates as PCPA1) and procarboxypeptidase A2 (hereinafter designates as PCPA2) is synthesized in the pancreas and externally secreted into duodenum and is restrictively cleaved by an action of trypsin to remove a pro-region (Non-patent reference 2: Biochem. J. (1992) 287, 299-303; non-patent reference 3: J. Biol. Chem. Vol. 270, No. 12, Issue of March 24, pp. 6651-6657, 1995).

Since CPA activity in body fluid such as serum and urine is changed due to disease of pancreas such as pancreatitis, CPA activity in body fluid is used as a pancreatic disease (pancreatitis) marker in a clinical laboratory test, and various assay methods for CPA activity have been developed. Assay methods for CPA activity include quantitatively assaying a product generated from substrates decomposed by CPA action as a result of a reaction with CPA and a specific substrate, and are so called based on principle of an enzymatic assay method. (Patent reference 1: JP-A-59-85299; patent reference 2: JP-A-59-66897; patent reference 3: JP-A-61-74600; patent reference 4: JP-A-11-127894; and patent reference 5: JP-A-09-313199).

Recently, in the human serum, since PCPA activity level in serum is found to be significantly higher in serum level of patients with pancreas cancer than in serum level of normal subjects in spite of having almost the same CPA activity level in serum of the patients with pancreas cancer and in serum of normal subjects, utilization of serum level of PCPA activity as a pancreas cancer marker and an assay method for PCPA activity level are proposed. A conventional assay method for PCPA activity is as follows. Pro-region of PCPA is removed by using protease such as trypsin, chymotrypsin, subtilisin, urokinase and clostripain to convert to activated CPA, then total activity of CPA converted from PCPA and originally existed CPA is assayed by an enzymatic method, thereby obtaining PCPA activity by subtracting originally existing CPA activity, which is obtained by assaying similarly without using protease from thus obtained total activity. (Non-patent reference 4: Peterson et al., Biochemistry 22: 3077-3082, 1983; Patent reference 6: Published Japanese translation of PCT international publication for patent application No. 2001-518791; and non-patent reference 5: Clinica Chimica Acta 292: 107-115, 2000).

JP-A-02-49162 (patent reference 7) discloses that an amount of an antibody against CPA (an anti-CPA antibody) in serum can be effectively used as a marker of lung cancer, ovarian cancer, laryngeal cancer, uterine cancer and liver cancer, and an immunoassay method for an anti-CPA antibody in serum using CPA derived from animals other than human is proposed. Further, in JP-A-10-75791 (patent reference 8), internal diagnosis using radioimmunoimaging of lung cancer is proposed wherein using an anti-CPA antibody light chain, which is reacted with CPA derived from bovine or porcine pancreas as well as reacting with lung cancer tissue, an antibody light chain and lung cancer tissue are subjected to direct reaction.

As described above, although relationship between CPA activity and pancreatitis, relationship between PCPA activity and pancreas cancer or relationship between an anti-CPA antibody or CPA and lung cancer is indicated, no report is found on relationship between CPA1, CPA2, PCPA1 or PCPA2 and pancreas cancer.

Further, although various assay methods for CPA and PCPA activities have been reported, there are no reports on specific assay method by differentiating CPA1, CPA2, PCPA1 and PCPA2 each other.

This is due to the fact that because conventional assay methods for CPA activity and PCPA activity are merely based on an enzymatic assay method, it is impossible to assay by differentiating CPA1 and CPA2 (or PCPA1 and PCPA2). In addition, since a conventionally known anti-CPA antibody is merely an antibody bonding to CPA, i.e. an antibody which specifically bonds both CPA1 and CPA2, and thus an antibody which specifically bonds to CPA1 and PCPA1, an antibody which specifically bonds to CPA2 and CPA2, an antibody which specifically bonds to PCPA1 and an antibody which specifically bonds to PCPA2 could not be obtained, therefore differential assaying of these enzymes could not be achieved even by using a conventionally known anti-CPA antibody.

### SUMMARY OF THE INVENTION

An aspect of the present invention is to provide an antibody which specifically binds to CPA1 and PCPA1, an antibody which specifically binds to CPA2 and PCPA2, an antibody which specifically binds to PCPA1 and an antibody which specifically binds to PCPA2, and a hybridoma producing a monoclonal antibody thereof. Another aspect of the present invention is to provide a method for the immunological assay, which can assay in highly precisely and simply one or more amounts selected from the group consisting of an amount of carboxypeptidase A1, an amount of carboxypeptidase A2, an amount of procarboxypeptidase A1, procarboxypeptidase A2 and a total amount of two or more thereof. Further aspect of the present invention is to provide a method for judgment of pancreas cancer highly precisely and simply.

The present invention constitutes the following:
(1) An antibody which specifically binds to CPA1 and PCPA1.
(2) An antibody which specifically binds to CPA2 and PCPA2.
(3) An antibody which specifically binds to PCPA1.
(4) An antibody which specifically binds to PCPA2.
(5) A method for immunologically assaying one or more amounts selected from an amount of CPA1, an amount of CPA2, an amount of PCPA1, an amount of PCPA2 and a total amount of two or more amounts selected therefrom, characterized by using one or more antibodies selected from a group consisting of an antibody which specifically bonds to CPA1 and PCPA1, an antibody which specifically bonds to CPA2 and PCPA2, an antibody which specifically bonds to PCPA1 and an antibody which specifically bonds to PCPA2.
(6) A method for judgment of pancreas cancer which comprises assaying one or more amounts selected from group consisting of an amount of carboxypeptidase A1, an amount of carboxypeptidase A2, an amount of procarboxypeptidase A1 and procarboxypeptidase A2 in a sample, and judging “whether the assay result indicates pancreas cancer or not” on the basis of thus obtained value.
(7) A kit for assaying one or more amounts selected from an amount of CPA1, an amount of CPA2, an amount of PCPA1, an amount of PCPA2 and a total amount of two or more amounts selected therefrom.
(8) A kit for judgment of pancreas cancer comprising one or more antibodies of any of above (1)-(4).

### DESCRIPTION OF THE SEVERAL VIEWS OF THE DRAWINGS

FIG. 1 is a result of Western blotting after electrophoresis of a cultured supernatant of the HEK-293 cell clone, to which PCPA1 gene is introduced, and a cultured supernatant of the HEK-293 cell clone, to which PCPA2 gene is introduced, obtained in Example 1.

FIG. 2 is a result of Western blotting after electrophoresis of a cultured supernatant of the HEK-293 cell clone, to which the base sequence of the pro-region of PCPA1 is introduced, and a cultured supernatant of the HEK-293 cell clone, to which the base sequence of the pro-region of PCPA2 is introduced, obtained in Example 3.

FIG. 3 is a result of evaluation on specificity of each anti-PCPA1 monoclonal antibody by EIA obtained in Example 3.

FIG. 4 is a result of evaluation on specificity of each anti-PCPA2 monoclonal antibody by EIA obtained in Example 3.

FIG. 5 is a calibration curve showing relationship between color development (absorbancy) and an amount of t-CPA1 (ng/ml), and a calibration curve showing relationship between color development (absorbancy) and an amount of PCPA1 (ng/ml) obtained in Example 4.

FIG. 6 is a calibration curve showing relationship between color development (absorbancy) and an amount of t-CPA2 (ng/ml), and a calibration curve showing relationship between color development (absorbancy) and an amount of PCPA2 (ng/ml) obtained in Example 4.

FIG. 7 is a drawing showing correlation between an amount of PCPA1 and an amount of t-CPA1 in each sample obtained in Example 4 and the correlation equation.

FIG. 8 is a drawing showing correlation between an amount of PCPA2 and an amount of t-CPA2 in each sample obtained in Example 4 and the correlation equation.

FIG. 9 is a drawing showing correlation between an amount of PCPA1 and an amount of PCPA2 in each sample obtained in Example 4 and the correlation equation.

FIG. 10 shows results (a graph showing distribution of amounts of PCPA1 by disease type) of comparison among an amount of PCPA1 in sera of healthy subjects, an amount of PCPA1 in sera of patients with chronic pancreatitis, an amount of PCPA1 in sera of patients with pancreas cancer by each stage, and an amount of PCPA1 in sera of patients with pancreas cancer (all patients) obtained in Example 4.

FIG. 11 shows results (a graph showing distribution of amounts of t-CPA1 by disease type) of comparison with an amount of t-CPA1 in sera of healthy subjects, an amount of t-CPA1 in sera of patients with chronic pancreatitis, an amount of t-CPA1 in sera of patients with pancreas cancer by each stage, and an amount of t-CPA1 in sera of patients with pancreas cancer (all atages) obtained in Example 4.

FIG. 12 shows results (a graph showing distribution of amounts of PCPA2 by disease type) of comparison with an amount of PCPA2 in sera of healthy subjects, an amount of PCPA2 in sera of patients with chronic pancreatitis, an amount of PCPA2 in sera of patients with pancreas cancer by each stage, and an amount of PCPA2 in sera of patients with pancreas cancer (all atages) obtained in Example 4.

FIG. 13 shows results (a graph showing distribution of amounts of t-CPA2 by disease type) of comparison with an amount of t-CPA2 in sera of healthy subjects, an amount of t-CPA2 in sera of patients with chronic pancreatitis, an amount of t-CPA2 in sera of patients with pancreas cancer by each stage, and an amount of t-CPA2 in sera of patients with pancreas cancer (all patients) obtained in Example 4.

### PREFERRED EMBODIMENTS OF THE INVENTION

Inventors of the present invention have studied extensively to obtain an antibody which specifically binds to CPA1 and PCPA1, an antibody which specifically binds to CPA2 and PCPA2, an antibody which specifically binds to PCPA1 and an antibody which specifically binds to PCPA2. As a result, we could obtain an antibody which specifically binds to CPA1 and PCPA1 without exhibiting reactivity to CPA2 and PCAP2, an antibody which specifically binds to CPA2 and PCPA2 without exhibiting reactivity to CPA1 and PCAP1, an antibody which specifically binds to PCPA1 without exhibiting reactivity to CPA1, CPA2 and PCPA2, and an antibody which specifically binds to PCPA2 without exhibiting reactivity to CPA1, CPA2 and PCPA1. Further, we have assayed an amount of CPA1, an amount of CPA2, an amount of PCPA1 and an amount of PCPA2 in samples derived from living body (e.g. blood, serum, plasma, etc.) by using these antibodies and studied relationship between these amounts or a total amount of two or more amounts selected therefrom and pancreas cancer, and as a result, we have found that the amount of CPA1, the amount of CPA2, the amount of PCPA1, the amount of PCPA2 and the total amount of two or more amounts selected there from were useful as a marker (indicator) for pancreas cancer, and judgment for “pancreas cancer or not” could be possible by using these amounts, and thereby completed the present invention.

In the present specification, a case “to judge” pancreas cancer or not includes a case “to diagnose” pancreas cancer or not based on the assay result of amounts of the above-described objects to be assayed.

1. An Antibody

1-1. An Antibody of the Present Invention

An antibody which specifically binds to CPA1 and PCPA1 of the present invention (hereinafter designates as a t-CPA1 specific antibody) is an antibody having reactivity to region, which is constant region existing commonly in both CPA1 and PCPA1 and is region not existing in any of CPA2 and PCPA2 (CPA1 specific region), (i.e. recognizing said specific region).

In other words, said antibody is an antibody having reactivity to each of CPA1 and PCPA1 (recognizing both CPA1 and PCPA1) but having substantially no reactivity to CPA2 and PCPA2 (not recognizing both CPA2 and PCPA2).

An antibody which specifically binds to CPA2 and PCPA2 of the present invention (hereinafter designates as a t-CPA2 specific antibody) is an antibody having reactivity to region, which is constant region existing commonly in both CPA2 and PCPA2 and is region not existing in any of CPA1 and PCPA1 (CPA2 specific region), (i.e. recognizing said specific region).

In other words, said antibody is an antibody having reactivity to each of CPA2 and PCPA2 (recognizing both CPA2 and PCPA2) but having substantially no reactivity to CPA1 and PCPA1 (not recognizing both CPA1 and PCPA1).

An antibody which specifically binds to PCPA1 of the present invention (hereinafter designates as PCPA1 specific antibody) is an antibody having reactivity to region, which is pro-region of PCPA1 and is region not existing in any of CPA1, CPA2 and PCPA2 (PCPA1 specific region), (i.e. recognizing said specific region).

In other words, said antibody is an antibody having reactivity to PCPA1 (recognizing PCPA1) but having substantially no reactivity to CPA1, CPA2 and PCPA2 (not recognizing CPA1, CPA2 and PCPA2).

An antibody which specifically binds to PCPA2 of the present invention (hereinafter designates as PCPA2 specific antibody) is an antibody having reactivity to region, which is pro-region of PCPA2 and is region not existing in any of CPA1, CPA2 and PCPA1 (PCPA2 specific region), (i.e. recognizing said specific region).

In other words, said antibody is an antibody having reactivity to PCPA2 (recognizing PCPA2) but having substantially no reactivity to CPA1, CPA2 and PCPA1 (not recognizing CPA1, CPA2 and PCPA1).

A t-CPA1 specific antibody, a t-CPA2 specific antibody, a PCPA1 specific antibody and a PCPA2 specific antibody of the present invention will sometimes be abbreviated totally as “a specific antibody of the present invention” below.

In the present specification, unless otherwise noted, “to have reactivity”, “to react”, “to bind” and “to recognize” are all used as synonym, indicating reactivity of an antibody (that is, to which it can bind). Further, description “a specifically bind” is used to mean only binding to specific object but no binding to others.

Origin of a specific antibody of the present invention is not limited, and can be any of a polyclonal antibody or a monoclonal antibody.

1-2. An Immunogen

CPA1, CPA2, PCPA1 and PCPA2 used as an immunogen for obtaining a specific antibody of the present invention can be obtained from pancreas of animals such as human, porcine, bovine, and the like or pancreatic secretory fluid thereof by known purification methods such as ion-exchange chromatography, hydrophobic interaction chromatography and gel filtration chromatography. Purification of protein can be performed by combining several known chromatographic techniques such as affinity chromatography using Sepharose beads coated with a t-CPA1 specific antibody, a t-CPA2 specific antibody, a PCPA1 specific antibody, a PCPA2 specific antibody, a t-CPA common antibody (an antibody binding to both CPA1 and CPA2) or a PCPA common antibody (an antibody binding to both PCPA1 and PCPA2).

Origin of an immunogen above is not especially limited and human, animals other than human (e.g. monkeys, mice, rats, rabbits, porcine, bovine, sheep, etc.), protista (e.g. nematode) and microorganisms (e.g. yeasts) can be used. Among others, human origin is preferable for an immunogen.

In the case that a t-CPA1 specific antibody and a t-CPA2 specific antibody are prepared, a mature protein corresponding to each is generally used as an immunogen, and in the case that a PCPA1 specific antibody and a PCPA2 specific antibody are prepared, a pro-protein corresponding to each is generally used as an immunogen (i.e. for preparing a t-CPA1 specific antibody, CPA1 is used as an immunogen; for preparing a t-CPA2 specific antibody, CPA2 is used as an immunogen; for preparing a PCPA1 specific antibody, PCPA1 is used as an immunogen; and for preparing a PCPA2 specific antibody, PCPA2 is used as an immunogen). However, when a t-CPA1 specific antibody and a t-CPA2 specific antibody are prepared, use of a pro-protein corresponding to each other is preferable (i.e. for preparing a t-CPA1 specific antibody, PCPA1 is preferably used as an immunogen, and for preparing a t-CPA2 specific antibody, PCPA2 is preferably used as an immunogen). This is due to the fact that because mature proteins of CPA1 and CPA2 are enzymes, there is such risk as to cause inflammatory reaction in an immunizing animal without generating immunoreaction, and further there is such risk as almost not to generate immunoreaction due to tolerance caused by a maturation enzyme (CPA1, CPA2, CPA, etc.) expressed by an immunizing animal itself. In addition, an antibody recognizing three-dimensional structure of a maturation enzyme (CPA1 or CPA2) can be prepared if a t-CPA1 specific antibody and a t-CPA2 specific antibody are prepared by using such a pro-protein as an immunogen.

Recombinant CPA1, recombinant CPA2, recombinant PCPA1 and recombinant PCPA2 obtained by known genetic engineering technique can also be used as an immunogen.

Origin of a recombinant (an immunogen) above is not especially limited, and a recombinant derived from human, animals other than human (e.g. monkeys, mice, rats, rabbits, porcine, bovine, sheep, etc.), protista (e.g. nematode) and microorganisms (e.g. yeasts) can be used. Among others, a recombinant of human origin is preferable, and a recombinant of human origin produced from cells of human origin (e.g. human embryonic kidney cells (HEK-293), human FL cells, HeLa cells (human cervical cancer), etc.) is especially preferable.

In the case that a t-CPA1 specific antibody and a t-CPA2 specific antibody are prepared, a maturation antigen corresponding to each is generally used as an immunogen, and in the case that a PCPA1 specific antibody and a PCPA2 specific antibody are prepared, a recombinant derived from a pro-antigen corresponding to each is generally used as an immunogen (i.e. for preparing a t-CPA1 specific antibody, recombinant CPA1 is used as an immunogen; for preparing a t-CPA2 specific antibody, recombinant CPA2 is used as an immunogen; for preparing a PCPA1 specific antibody, recombinant PCPA1 is used as an immunogen; and for preparing a PCPA2 specific antibody, recombinant PCPA2 is used as an immunogen). However, when a t-CPA1 specific antibody and a t-CPA2 specific antibody are prepared, using a recombinant derived from a pro-protein corresponding to each other is preferable (i.e. for preparing a t-CPA1 specific antibody, recombinant PCPA1 is preferably used as an immunogen, and for preparing a t-CPA2 specific antibody, recombinant PCPA2 is preferably used as an immunogen). This is due to the fact that because mature proteins of CPA1 and CPA2 are enzymes, there is such risk as to cause inflammatory reaction in an immunizing animal without generating immunoreaction, and further there is such risk as almost not to generate immunoreaction due to tolerance caused by a maturation enzyme (CPA1, CPA2, CPA, etc.) expressed by an immunizing animal itself. In addition, an antibody recognizing three-dimensional structure of a maturation enzyme (CPA1 or CPA2) can be prepared if a t-CPA1 specific antibody and a t-CPA2 specific antibody are prepared by using such a pro-protein as an immunogen.

In the case of preparing recombinant CPA1 by the genetic engineering technique, for example PCR is conducted in conventional means by using a reverse primer designed from an optional position selected from 3′-terminal region of the base sequence (SEQ ID NO: 5) of human CPA1 cDNA (GenBank accession No. BT007313) coding CPA1 gene, a forward primer designed from an optional position in region from 5′-terminal to the initiation codon of SEQ ID NO: 5, and a template, which is a cDNA library (human leukocyte cDNA, etc.) containing human CPA1 cDNA (GenBank accession No. BT007313), and DNA fragment of the sequence containing the objective CPA1 gene or the gene fragment thereof (e.g. the base sequence of 331-1260 bp of SEQ ID NO: 5) is amplified, then thus obtained DNA fragment is inserted into a preferable vector DNA by conventional means to obtain recombinant plasmid.

Further, in the case of preparing recombinant CPA2, recombinant PCPA1 or recombinant PCPA2 by the genetic engineering technique, using the base sequence of human CPA2 cDNA (GenBank accession No. BT007403) (SEQ ID NO: 11), the base sequence of human PCPA1 cDNA (GenBank accession No. BT007313) (SEQ ID NO: 5) or the base sequence of human PCPA2 cDNA (GenBank accession No. BT007403) (SEQ ID NO: 11) in place of human CPA1 cDNA, DNA fragment of a sequence containing the objective CPA2 gene or a gene fragment thereof (e.g. the base sequence of 331-1260 bp in SEQ ID NO: 11), DNA fragment of a sequence containing the objective PCPA1 gene or a gene fragment thereof (e.g. the base sequence of 48-1260 bp in SEQ ID NO: 5), or DNA fragment of a sequence containing objective PCPA2 gene or a gene fragment thereof (e.g. the base sequence of 48-1254 bp in SEQ ID NO: 11), which are amplified by the same way as above, is inserted into a preferable vector DNA by conventional means to obtain recombinant plasmid.

A base sequence of DNA fragment inserted in a recombinant vector is analyzed, and the insertion of a sequence containing objective CPA1 gene or the fragment thereof, CPA2 gene or the fragment thereof, PCPA1 gene or the fragment thereof, or PCPA2 gene or the fragment thereof is confirmed.

A DNA fragment (including a sequence containing CPA1 gene, CPA2 gene, PCPA1 gene, PCPA2 gene, or fragments thereof) inserting in a vector can be used as it is, or by optionally digesting with a restriction enzyme, or by adding a linker depending on use object.

An expression vector used herein includes a plasmid vector, phage vector and a viral vector.

Specifically, a plasmid vector such as the p3× FLAG CMV vector series (SIGMA-ALDRICH Co.), the pcDNA3.1/myc-His vector (Invitrogen Corp.), pUC119 (TAKARA BIO INC) and pBluescript II KS+ (Stratagene Inc.), a bacteriophage vector such as λ EMBL 3 (Stratagene Inc.) and λ DASHII (Funakoshi Co.), and a cosmid vector such as Charomid DNA (Wako Pure Chemical Ind.) and Lorist6 (Wako Pure Chemical Ind.) are included.

Further, a plasmid derived from E. coli (e.g. pTrc99A, pKK223 and pET3a), a plasmid derived from B. subtilis (e.g. pUB110, pTP5and pC194), a plasmid derived from yeast (e.g. pSH19 and pSH15), bacteriophage such as λ-phage, animal virus such as retrovirus, vaccinia virus and baculovirus, pA1-11, pXT1, pRc/CMV, pRc/RSV, pcDNA I/Neo, p3× FLAG-CMV-14, etc. can be used.

For simplifying detection and purification, objective CPA1 protein, CPA2 protein, PCPA1 protein or PCPA2 protein may optionally be expressed as fused protein with other tag peptide or protein. Examples of tag peptide to be fused are FLAG-tag, 3× FLAG-tag, His6-tag, etc. Examples of protein are β-galactosidase (β-gal), green fluorescent protein (GFP) and maltose binding protein (MBP).

Practically, a product of PCR obtained by using a primer, in which a sequence coding the above-described tag peptide is designed in both sides of the open reading frame, is subcloned in an expression vector, or a sequence coding a tag peptide is inserted between said gene and an expression vector, or an expression vector containing a sequence previously coding tag peptide or a protein is used, then CPA1 protein, CPA2 protein, PCPA1 protein or PCPA2 protein can be expressed as fused protein with peptide or protein. For example, when the pcDNA3.1/myc-His vector (Invitrogen Inc.), to which myc epitope tag gene and His tag gene are inserted, is used as an expression vector, and a sequence containing CPA1 gene, CPA2 gene, PCPA1 gene, PCPA2 gene or fragment thereof is inserted into the upstream of the tag, expression of CPA1 gene, CPA2 gene, PCPA1 gene or PCPA2 gene in the upstream region can be confirmed by observation of expression of His tag or myc epitope tag.

Using thus obtained recombinant vector, a transformant can be obtained by transforming, for example, Escherichia coli (E. coli) K-12 (American Type Culture Collection, ATCC), E. coli JM109 (K.K. Nippon Gene), DH5α (K.K. Nippon Gene) or VCS257, preferably E. coli K-12.

Transformation can be performed, for example, according to a method of D. M. Morrison (“Method in Enzymology”, 68, 326-331, 1979).

After mass culture of thus obtained transformant, plasmid is recovered from thus obtained transformant by conventional means.

When a transformant, a host of which is E. coli, is cultured, medium used for culturing a transformant is preferably liquid culture medium, and carbon sources, nitrogen sources, inorganic materials, and the like necessary for growth of a transformant can be contained. Examples of carbon sources are glucose, dextrin, soluble starch, sucrose, etc. Examples of nitrogen sources are inorganic or organic substances such as ammonium salts, nitrates, corn steep liquor, peptone, yeast extract, casein, meat extract, soybean cake, potato extract liquid, etc. Examples of inorganic salt are calcium chloride, sodium dihydrogen phosphate, magnesium chloride, etc. Further, vitamins, growth factors, etc. may also be added. Preferable medium pH is pH 5-8.

Examples of medium for culturing a transformant, in which a host of a transformant is E. coli, are LB medium, 2×YT medium and M9 medium (Miller, J. Experiments in Molecular Genetics, 431-433, Cold Spring Harbor Laboratory, New York, 1972). If necessary, an agent such as isopropyl-β-D-thiogalactopyranoside (IPTG) and 3β-indolylacrylate can be added.

Thus obtained expressed plasmid is introduced into a preferable host cell.

Example of a host cell used for obtaining a transformant is preferably animal cells. Especially, cells of human origin are preferable.

Examples of animal cells are cells of human origin such as human embryonic kidney cells (HEK-293), human FL cells, HeLa (human cervical cancer) cells, and cells of animal origin other than human such as African green monkey cell COS-1, African green monkey cell COS-7, Vero, Chinese hamster cell CHO (e.g. CHO-K1), dhfr gene deficient Chinese hamster cell CHO, mouse L cell, mouse AtT-20, mouse myeloma cell, rat GH3, etc. Further examples of cells of human origin include various normal human cells such as liver cells, spleen cells, nerve cells, glial cells, pancreatic β cells, bone marrow cells, mesangial cells, Langerhans cells, epidermal cells, epithelial cells, endothelial cells, fibroblasts, fibrocytes, muscle cells, adipocytes, immunocytes (e.g. macrophage, T cell, B cell, NK cell, mast cell, neutrophil, basophil, eosinophil and monocyte), megakaryocytes, synovial cells, chondrocytes, osteocytes, osteoblasts, osteoclasts, mammary cells, hepatocytes or interstitial cells, or progenitor cells of these cells, stem cells or cancer cells, etc.

For introducing thus obtained plasmid into animal cells, a method, for example, described in “Cell Technology”, Supplement Volume 8, “New cell technology experiments, a protocol”, 263-267, 1995 (published by Shujunsha) and Virology, 52, 456, 1973, can be applied.

Further, using commercially available reagents for transformation, the process may be achieved according to the manufacturer's protocol. Example thereof is an electroporation using GenePulser Xcell (Bio-Rad, Inc.).

As for a method for confirming acquisition of a transformant carrying expression plasmid, in which a sequence containing objective CPA1 gene sequence, CPA2 gene sequence, PCPA1 gene sequence, PCPA2 gene sequence or fragments thereof is integrated, a method utilizing drug resistant gene existing previously in a vector used for obtaining an expression vector, detecting drug resistance of a transformant and confirming the resistance are included. For example, when the pcDNA3.1/myc-His vector is used as an expression vector, the vector has neomycin resistant gene (neo). Consequently, for example, a method includes, wherein cells are cultured in a medium added with Geneticin and the cultured growth cells (neomycin resistant strain) are confirmed as a transformant carrying an introduced sequence integrated with objective CPA1 gene, CPA2 gene, PCPA1 gene, PCPA2 gene or fragments thereof.

For confirming production of objective CPA1 protein, CPA2 protein, PCPA1 protein, PCPA2 protein or fragments thereof (hereinafter designates as “recombinant CPA1 protein”, “recombinant CPA2 protein”, “recombinant PCPA1 protein” or “recombinant PCPA2 protein”) (expression of CPA1 gene, CPA2 gene, PCPA1 gene or PCPA2 gene) in a transformant, when recombinant CPA1 protein, recombinant CPA2 protein, recombinant PCPA1 protein or recombinant PCPA2 protein is secreted into cultured medium of a transformant, cultured medium (cultured supernatant) is, for example, subjected to conventional immunoassay (dot Western blotting, Western blotting, etc.) using an antibody to tag peptide and a transformant, which is confirmed to express tag peptide in the cultured supernatant, is selected, then a transformant expressing objective recombinant CPA1 protein, recombinant CPA2 protein, recombinant PCPA1 protein or recombinant PCPA2 protein can be obtained.

When protein is not secreted in culture medium of a transformant, for example, in the case of expressing recombinant CPA1 protein, recombinant CPA2 protein, recombinant PCPA1 protein or recombinant PCPA2 protein as transmembrane protein, etc., a transformant is treated with conventional cell destruction or lysis (e.g. treatment with a homogenizer, treatment with a membrane lytic agent such as a proper surfactant, etc.) to obtain lysate thereof. The lysate is subjected to conventional immunoassay in the same way as in the case of the above cultured supernatant, and a transformant expressing objective recombinant CPA1 protein, recombinant CPA2 protein, recombinant PCPA1 protein or recombinant PCPA2 protein can be selectively obtained.

Further, cultured supernatant or lysate is subjected to conventional immunoassay such as ELISA, etc. using an anti-t-CPA1 antibody, an anti-t-CPA2 antibody, an anti-PCPA1 antibody, an anti-PCPA2 antibody or an antibody against tag peptide, then a transformant with confirmed existence of recombinant CPA1 protein, recombinant CPA2 protein, recombinant PCPA1 protein or recombinant PCPA2 protein is selected to obtain a transformant which expresses objective recombinant CPA1 protein, recombinant CPA2 protein, recombinant PCPA1 protein or recombinant PCPA2 protein.

CPA1 protein, CPA2 protein, PCPA1 protein, PCPA2 protein or fragments thereof can be obtained by culturing a transformant, in which expression plasmid, integrated with a sequence containing CPA1 gene, CPA2 gene, PCPA1 gene, PCPA2 gene or fragments thereof as obtained above, is introduced, in nutrient medium, and produced recombinant CPA1 protein, recombinant CPA2 protein, recombinant PCPA1 protein or recombinant PCPA2 protein.

When a transformant is cultured in animal cell host, examples of media used are GIT medium (Wako Pure Chemical Ind.), Opti-Mem medium (GIBCO), D-MEM (GIBCO), e.g. MEM medium containing about 5-20% fetal calf serum (FCS) (Science, 122: 501, 1952), Dulbecco modified Eagle's medium (DME medium: Virology, 8: 396, 1959), RPMI-1640 medium (J. Am. Med. Ass. 199: 519, 1967), medium 199 (Proc. Soc. Biol. Med. 73: 1, 1950), etc. Preferable pH is about pH 6-8. Culture is generally performed at about 30° C.-40° C. for about 15-120 hours, under 5% CO₂, and aeration and spinner are added, if necessary.

When CPA1 protein, CPA2 protein, PCPA1 protein or PCPA2 protein are secreted into culture medium, after terminating culture, a transformant and the cultured supernatant are separated by a known method and cultured supernatant was collected. When protein is not secreted in culture medium of a transformant, for example, in the case of expressing recombinant CPA1 protein, recombinant CPA2 protein, recombinant PCPA1 protein or recombinant PCPA2 protein as transmembrane protein, and the like, a transformant is treated with conventional cell destruction or lysis as described above to obtain lysate thereof. If necessary, the lysate is optionally subjected to centrifugation, and the like before purification treatment of recombinant CPA1 protein, recombinant CPA2 protein, recombinant PCPA1 protein or recombinant PCPA2 protein.

Purification of thus obtained recombinant CPA1 protein, recombinant CPA2 protein, recombinant PCPA1 protein or recombinant PCPA2 protein in cultured medium or lysate can be achieved by adequately combining with known separation and purification methods. Examples of known separation and purification methods include a method applying difference in solubility such as salting out and aolvent precipitation, a method applying mainly difference in molecular weight such as dialysis, ultrafiltration, gel filtration and SDS-polyacrylamide gel electrophoresis, a method applying difference in charge such as ion exchange chromatography, a method applying specific affinity such as affinity chromatography, a method applying difference in hydrophobicity such as reverse high performance liquid chromatography, a method applying difference in isoelectric point such as isoelectric focusing, etc.

Existence of thus separated and purified recombinant CPA1 protein, recombinant CPA2 protein, recombinant PCPA1 protein or recombinant PCPA2 protein can be confirmed by assaying with ELISA using an anti-t-CPA1 antibody, an anti-t-CPA2 antibody, an anti-PCPA1 antibody, an anti-PCPA2 antibody or an antibody against tag peptide.

A method for obtaining objective recombinant CPA1 protein will be explained more specifically as follows.

At first, in conventional means, a reverse primer is designed from an optional position selected from 3′-terminal region of the base sequence (SEQ ID NO: 5) of human CPA1 cDNA (GenBank accession No. BT007313) coding CPA1 gene, and a forward primer is designed from an optional position in the region from 5′-terminal to initiation codon of SEQ ID NO: 5. Conventional PCR is conducted by using a reverse primer, a forward primer and a template, which is a cDNA library (human leukocyte cDNA, etc.) containing human CPA1 cDNA (GenBank accession No. BT007313), to amplify the sequence containing the objective CPA1 gene (the base sequence of 331-1260 bp of SEQ ID NO: 5) or gene fragments thereof. Reaction may be performed for 15-40 cycles.

Thus obtained DNA fragments are digested, if necessary, by proper restriction enzymes such as HindIII, NotI, etc. and are inserted into a proper restriction site in an expression vector such as the pcDNA3.1/myc-His vector. Drug resistant gene such as neomycin resistance (neo) may optionally be inserted into an expression vector to confirm whether a transformant integrates objective gene. Further, myc epitope tag gene and sequence coding tag peptide such as His tag gene and tag peptide may be contained in advance.

Using thus obtained recombinant vector, a host such as bacteria, e.g. E. coli, is transformed to obtain a transformant. After mass culture of a transformant, plasmid is recovered by using conventional means such as cesium chloride density-gradient centrifugation and a plasmid purification kit (Qiagen Inc.). Thus obtained expression plasmid is introduced into a proper host cell such as the HEK-293 cell by electroporation using, for example, GenePulser Xcell (Bio-Rad Inc.). Then a transformant integrated with objective gene is selected by using drug resistance and is subjected to cloning by conventional means such as limiting dilution.

Selection of clone consisting of a transformant which purify objective recombinant CPA1 protein can be performed by the following means. In the case that protein is secreted in cultured supernatant of a transformant, a transformant subjecting to cloning is cultured, and the positive clone, which secretes recombinant CPA1 protein in cultured supernatant, is selected by conventional means such as dot Western blotting by using an antibody against tag peptide to obtain cell line which expresses recombinant CPA1 protein.

In the case that recombinant CPA1 protein is not secreted in cultured medium of a transformant as described above, thus obtained transformant is treated by conventional means of cell destruction or lysis as described above to obtain lysate. Lysate is treated by the same way as in the above culture supernatant. For example a positive clone, which produces recombinant CPA1 protein, is selected by an conventional means such as dot Western blotting by using an antibody against tag peptide to obtain cell line which expresses recombinant CPA1 protein.

For obtaining recombinant CPA1 protein in the case of excreting protein in culture supernatant of a transformant, cell strain which can stably express recombinant CPA1 protein is cultured to 100% confluent state in medium such as GIT medium (Wako Pure Chemical Ind.) and, if necessary, medium is changed to other medium such as Opti-MEM medium (GIBCO), with repeating medium exchange to obtain cultured supernatant. Cultured supernatant recovered is subjected to a conventional means for production and concentration of protein such as centrifugation, ultrafiltration, affinity chromatography, etc., then recombinant CPA1 protein can be obtained.

In the case that protein is not secreted in cultured medium of a transformant, cell strain which can stably express recombinant CPA1 protein is cultured, and the cell is treated by conventional means of cell destruction or lysis as described above to obtain lysate. Lysate is treated by the same way as in the above culture supernatant. For example, cultured supernatant recovered is subjected to a conventional means for production and concentration of protein such as centrifugation, ultrafiltration, affinity chromatography, etc., and then recombinant CPA1 protein can be obtained.

For obtaining recombinant CPA2 protein, recombinant PCPA1 protein or recombinant PCPA2 protein, in the above method, the base sequence of Human CPA2 cDNA (GenBank accession No. BT007403) (SEQ ID NO: 11), Human PCPA1 cDNA (GenBank accession No. BT007313) (SEQ ID NO: 5) or Human PCPA2 cDNA (GenBank accession No. BT007403) (SEQ ID NO: 11) is used in place of Human CPA1 cDNA, and a DNA fragment of a sequence containing CPA2 gene or gene fragments thereof (e.g. the base sequence of 331-1260 bp in SEQ ID NO: 11), a DNA fragment of a sequence containing PCPA1 gene or gene fragments thereof (e.g. the base sequence of 48-1254 bp in SEQ ID NO: 5) and a DNA fragment of a sequence containing PCPA2 gene or gene fragments thereof (e.g. the base sequence of 48-1251 bp in SEQ ID NO: 11) amplified by the same way as above are preferably used.

1-3. A Manufacturing Method for an Antibody

A method for preparing a CPA1 specific polyclonal antibody, a CPA2 specific polyclonal antibody, a PCPA1 specific polyclonal antibody and a PCPA2 specific polyclonal antibody (hereinafter sometimes totally designated as “a specific polyclonal antibody of the present invention”) includes a conventional method wherein animals such as equine, bovine, sheep, rabbit, goat, guinea pig, rat, mouse, etc. are immunized by using CPA1, CPA2, PCPA1, PCPA2 or fragments thereof obtained by the above method according to a method described in “Introduction to Immunological Experiments” 2nd Ed. Matsuhashi, N. et al. K.K Gakkai Shuppan Center, 1981.

Further, a method for preparing a CPA1 specific monoclonal antibody, a CPA2 specific monoclonal antibody, a PCPA1 specific monoclonal antibody and a PCPA2 specific monoclonal antibody (hereinafter sometimes totally designated as “a specific monoclonal antibody of the present invention”) includes the following: Immunized cells, for example spleen cells and lymphocytes of animals such as rat and mouse immunized by an immunogen of CPA1, CPA2, PCPA1, PCPA2 or fragments thereof obtained by the above method, and cells having permanently proliferating nature such as myeloma cells are fused by known cell fusion technique developed by Koehler and Milstein (Nature, 256, 495, 1975) to prepare hybridoma, selected hybridoma, which produces a monoclonal antibody specific to assay objects, and hybridoma is cultured in medium or is administered intraperitoneally in animals to produce an antibody in ascites, then an objective monoclonal antibody is yielded from cultured material or ascites; cells, which produce an antibody having the above-described properties, are prepared by a known method applied with gene recombination technique (Eur. J. Immunol., 6, 511, 1976), and cells are cultured to obtain an objective monoclonal antibody.

A method for obtaining a specific monoclonal antibody of the present invention by using recombinant CPA1 protein, recombinant CPA2 protein, recombinant PCPA1 protein or recombinant PCPA2 protein obtained by the method above is exemplified for more specifically as follows.

Recombinant CPA1 protein, recombinant CPA2 protein, recombinant PCPA1 protein or recombinant PCPA2 protein obtained by the method above and an adjuvant such as a complete (or incomplete) Freund adjuvant are mixed to prepare a suspension. The suspension is subcutaneously, intravenously or intraperitoneally administered to animals in an adequate amount, generally 0.1-100 μg in one dose for an animal, preferably 0.1-10 μg as an amount of protein in recombinant CPA1 protein, recombinant CPA2 protein, recombinant PCPA1 protein or recombinant PCPA2 protein, for every 1-5 weeks, preferably for every 2-5 weeks, generally 3-10 times, preferably 3-8 times to immunize the animals. After immunization, blood was collected from the animals, and serum thereof is confirmed to react with CPA1, CPA2, PCPA1 or PCPA2 by a known method such as solid phase enzyme immunoassay (ELISA) using a solid phase binding CPA1, CPA2, PCPA1 or PCPA2 with the insoluble carrier. After confirmation, spleen is extracted from the immunized animals in 3-4 days from the final immunization, and spleen cells are prepared by a conventional method. Thus obtained spleen cells and myeloma cells such as NS-1, Sp2 and X63 are fused according to a conventional method, and the fused cells are selected, as conventionally, by using HAT medium. Selected fused cells are cultured and cultured supernatant is subjected to conventional ELISA, indirect fluorescent an antibody technique, SDS-polyacrylamide electrophoresis, and is then subjected to Western blot immunoassay using a polyvinylidene difluoride (PVDF) membrane to further select cells which produce a specific antibody antibody of the present invention having the above-described properties. Subsequently, cloning with limiting dilution was performed for several times, and cells, which can be recognized to stably produce a highly potential antibody, are selected as a specific monoclonal antibody producing hybridoma strain.

Further, the slected specific antibody of the present invention is confirmed not to react with any protein other than objective protein by a known immunological assay method such as solid phase wnzyme immunoassay (ELISA) using a solid phase binding any protein other than objective protein or a conventional means assaying an antigenic specificity of antibody such as flow cytometry and immunoprecipitation to select as a specific monoclonal antibody producing hybridoma strain.

In the above-described solid phase enzyme immunoassay (ELISA): in the case that a selected specific monoclonal antibody of the present invention is a t-CPA1 specific monoclonal antibody (i.e. in the case that objective protein is CPA1 and PCPA1), a solid phase, in which CPA2 and PCPA2 are bound to an insoluble carrier, is used; in the case of a t-CPA2 specific monoclonal antibody (i.e. in the case that objective protein is CPA2 and PCPA2), a solid phase, in which CPA1 and PCPA1 are bound to an insoluble carrier, is used; in the case of a PCPA1 specific monoclonal antibody (i.e. in the case that objective protein is PCPA1), a solid phase, in which CPA1, CPA2 and PCPA2 are bound to an insoluble carrier, is used; or in the case of a PCPA2 specific monoclonal antibody (i.e. in the case that objective protein is PCPA2), a solid phase, in which CPA1, CPA2 and PCPA1 are bound to an insoluble carrier, is used; and each specific monoclonal antibody should be confirmed not to react with any protein other than objective protein.

Thus obtained hybridoma is intraperitoneally injected by a conventional means to produce a specific antibody of the present invention in ascites. Ascites are collected and purified by conventional purification methods used in this field such as ammonium sulfate salting, dialysis using buffer such as phosphate buffer, DEAE cellulose chromatography and affinity chromatography using CPA1, CPA2, PCPA1 or PCPA2 to obtain a specific monoclonal antibody of the present invention (a t-CPA1 specific monoclonal antibody, a t-CPA2 specific monoclonal antibody, a PCPA1 specific monoclonal antibody or a PCPA2 specific monoclonal antibody of the present invention).

1-4. Effect of a Specific Antibody of the Present Invention

A t-CPA1 specific antibody of the present invention has specifically binding property to CPA1 and PCPA1; a t-CPA2 specific antibody of the present invention has specifically binding property to CPA2 and PCPA2; a PCPA1 specific antibody of the present invention has specifically binding property to PCPA1; and a PCPA2 specific antibody of the present invention has specifically binding property to PCPA2, and these antibodies are novel antibodies which have never been known until now. Consequently, performing an immunoassay using such specific antibodies of the present invention, the following effects can be obtained.
(1) By performing immunoassay using a t-CPA1 specific antibody, a total amount of CPA1 and PCPA1 (t-CPA1 amount) can be simply assayed specifically (differentiating from CPA2 and PCPA2).
(2) By performing immunoassay using a t-CPA2 specific antibody, a total amount of CPA2 and PCPA2 (t-CPA2 amount) can be simply assayed specifically (differentiating from CPA1 and PCPA1).
(3) By performing immunoassay using a PCPA1 specific antibody, an amount of PCPA1 can be simply assayed specifically (differentiating from CPA1, CPA2 and PCPA2).
(4) By performing immunoassay using a PCPA1 specific antibody, an amount of PCPA2 can be simply assayed specifically (differentiating from CPA1, CPA2 and PCPA1).
(5) By performing immunoassay using a t-CPA1 specific antibody and a t-CPA2 specific antibody, a total amount of CPA1, CPA2, PCPA1 and PCPA2 (total t-CPA amount) can be simply assayed.
(6) By performing immunoassay using a PCPA1 specific antibody and a PCPA2 specific antibody, a total amount of PCPA1 and PCPA2 (cumulative amount of PCPA) can be simply assayed specifically (differentiating from CPA1 and CPA2).
(7) By performing immunoassay using a t-CPA1 specific antibody and a PCPA1 specific antibody, an amount of CPA1 can be assayed simply.
(8) By performing immunoassay using a t-CPA2 specific antibody and a PCPA2 specific antibody, an amount of CPA2 can be simply assayed.
(9) By performing immunoassay using a t-CPA1 specific antibody, a t-CPA2 specific antibody, a PCPA1 specific antibody and a PCPA2 specific antibody, a total amount of CPA1 and CPA2 (total CPA amount) can be simply assayed.

Determination of subclass of a monoclonal antibody may preferably be performed by a known method such as double immunodiffusion (“Summary of Clinical Laboratory Tests” 30th Printing, p. 842-843m Kanahara Publ. Co.).

Specific examples of a specific antibody of the present invention includes as follows. Hybridomas which produce such specific antibodies were deposited in the International Patent Organisms Depositary, National Institute of Advanced Industrial Science and Technology, AIST Tsukuba Central 6, 1-1, Higashi 1-Chome, Tsukuba-shi, Ibaraki-ken, 305-8566 Japan, on the following depository date and acceptance No.
a t-CPA1 specific monoclonal antibody: A monoclonal antibody 1-7-5 produced by hybridoma cell strain CPA7-5 (FERM ABP-10128)
a t-CPA1 specific monoclonal antibody: A monoclonal antibody 2-14-76 produced by hybridoma cell strain CPA14-76 (FERM ABP-10129)
a PCPA1 specific monoclonal antibody: A monoclonal antibody 2-15-95 produced by hybridoma cell strain CPA15-95 (FERM ABP-10130)

An antibody can be used in a form of an antibody molecule itself, or if necessary, it may be used in a form of F(ab′)₂, Fab′ or Fab after digesting an antibody by using enzyme such as pepsin, papain, etc.

2. An Assay Method

2-1. An Assay Method of the Present Invention

As described above, by performing immunoassay using one or more antibodies selected from a specific antibody of the present invention, one or more amounts selected from an amount of CPA1, an amount of CPA2, an amount of PCPA1, an amount of PCPA2 and total amount of two or more amounts selected from these amounts (a total amount of CPA1 and PCPA1 (t-CPA1 amount); a total amount of CPA2 and PCPA2 (t-CPA2 amount); a total amount of CPA1, CPA2, PCPA1 and PCPA2 (total t-CPA amount); a total amount of PCPA1 and PCPA2 (tatol PCPA amount); and a total amount of CPA1 and CPA2 (totale CPA amount)) can be assayed.

Relationship between specific antibodies of the present invention and objectives which can be assayed and calculated is shown in Table 1.

TABLE 1

| | An antibody used | | | | |
|---|---|---|---|---|---|
| | t-CPA1 | t-CPA2 | PCPA1 | PCPA2 | |
| | SpAb | SpAb | SpAb | SpAb | Objectives to be assayed |
| 1 | ∘ | — | — | — | Total amount of CPA1 and |
| | | | | | PCPA1 (t-CPA1 amount) |
| 2 | — | ∘ | — | — | Total amount of CPA2 and |
| | | | | | PCPA2 (t-CPA2 amount) |
| 3 | — | — | ∘ | | Amount of PCPA1 |
| 4 | — | — | — | ∘ | Amount of PCPA2 |
| 5 | ∘ | ∘ | — | — | Amount of t-CPA1 & amount |
| | | | | | of t-CPA2; Total amounts |
| | | | | | of CPA1, CPA2, PCPA1 and |
| | | | | | PCPA2 (total t-CPA amount) |
| 6 | — | — | ∘ | ∘ | Amount of PCPA1 & amount |
| | | | | | of PCPA2; Total amounts |
| | | | | | of PCPA1 and PCPA2 (total |
| | | | | | PCPA amount) |
| 7 | ∘ | — | ∘ | — | Amount of t-CPA1 & amount |
| | | | | | of PCPA1; Amount of CPA1 |
| 8 | — | ∘ | — | ∘ | Amount of t-CPA2 & amount |
| | | | | | of PCPA2; Amount of CPA2 |
| 9 | ∘ | ∘ | ∘ | ∘ | Amount of t-CPA1, amount |
| | | | | | of PCPA1, amount of |
| | | | | | t-CPA2 & amount of PCPA2; |
| | | | | | Amount of CPA1 & amount |
| | | | | | of CPA2; Total CPA amount |

SpAb: specific antibody

Assay of the present invention can be performed by a known immunoassay method except that one or more antibodies selected from a specific antibody of the present invention is used. Examples of such immunoassay methods are known immunological methods used in this field concerned, for example: enzyme immunoassay (EIA); enzyme-linked immunosorbent assay (ELISA); radioimmunoassay (RIA); chemiluminescence immunoassay (CLEIA); fluoroimmunoassay (FIA); immuno-polymerase chain reaction (Furuya, D. et al. J. Immunol. Methods, 238, 173-180, 2000); assay method using high-pressure liquid chromatography (HPLC) (JP-A-09-301995); a method using a hybrid enzyme as a labeled substance (JP, 2002-65283,A); a method using fluorescence resonance energy transfer (Kaj, B. et al. Ckin. Chem. 45, 855-861, 1999); an assay method based on turbidity generated by an antigen-antibody reaction (e.g. immunonephelometry, nephelometric immunoassay, latex fixation, etc.) (“Summary of Clinical Laboratory Tests” 30th Ed. 2nd Printing, p. 851-856, Kanahara Publ. Co.; “New Cases of Immunoassay and Application to Development of Diagnostic Reagents and Therapeutic Agents, p. 103-187, Keiei Kyoiku Pub. Co.; JP-A-10-197533; JP, 2002-365296, A; JP-A-58-11575; JP-A-10-123137; JP-A-10-197533), etc. Among others, chemiluminescence immunoassay (CLEIA) and fluoroimmunoassay (FIA) are preferable, and assay can be performed with high sensitivity and simplicity.

Further, known immunoassay includes electrophoresis applying an antigen-antibody reaction such as capillary electrophoresis, capillary chip electrophoresis, capillary column chromatography, immunoassay utilizing surface plasmon resonance, etc. (Jpn. Patent No. 3070418, JP-A-10-512371(1998), WO 02/082083, JP-A-10-090271, etc.)

The above-mentioned principle of assay methods can be any of sandwich immunoassay, a competitive method and a double-antibody technique. Further, assaying under heterogeneous condition with BF separation or assaying under homogeneous condition without BF separation may be possible. Among others, since objects to be assayed can be assayed with high sensitivity and simplicity, immunoassay of sandwich immunoassay using one or more antibodies selected from a specific antibody of the present invention and an antibody which binds to one or more enzymes selected from CPA1, CPA2, PCPA1 and PCPA2 (hereinafter designates as a CPA/PCPA binding antibody) is preferable.

2-2. Sandwich Immunoassay

An example of a homogeneous assay method includes: Using (1) one or more antibodies selected from a specific antibody of the present invention and (2) one or more antibodies selected from a CPA/PCPA binding antibody, and using a labeled antibody prepared from either one of these antibodies labeled with a labeling substance, which can change specific signaling level according to condition whether a complex is formed or not, sandwich immunoassay is conducted according to the above-described assay method to form one or more complexes selected from (i) a complex with (a) an unlabeled antibody, (b) CPA1 in a sample and (c) a labeled antibody, (ii) a complex with (a) an unlabeled antibody, (b) CPA2 in a sample and (c) a labeled antibody, (iii) a complex with (a) an unlabeled antibody, (b) PCPA1 in a sample and (c) a labeled antibody, and (iv) a complex with (a) an unlabeled antibody, (b) PCPA2 in a sample and (c) a labeled antibody, thereby assaying an amount of CPA1, an amount of CPA2, an amount of PCPA1, an amount of PCPA2 and a total amount of two or more amounts selected from these amounts (a total amount of CPA1 and PCPA1 (t-CPA1 amount); a total amount of CPA2 and PCPA2 (t-CPA2 amount); a total amount of CPA1, CPA2, PCPA1 and PCPA2 (total t-CPA amount); a total amount of PCPA1 and PCPA2 (total PCPA amount); and a total amount of CPA1 and CPA2 (total CPA amount)) in a sample, on the basis of the changed signal level depending on the amount of the complex.

In the above assay, any of an antibody (1) and an antibody (2) can be labeled. In the case that an antibody (1) and an antibody (2) are antibodies having the same properties (i.e. in the case of using two types of particular antibodies among specific antibodies of the present invention), these antibodies are different in each epitope.

Further, an example of a heterogeneous assay method using an insoluble carrier includes the following. Using (1) one or more antibodies selected from a specific antibody of the present invention and (2) one or more antibodies selected from a specific antibody, a t-CPA common antibody and a PCPA common antibody, either one of an antibody in these antibodies is used as an immobilized antibody immobilized to an insoluble carrier and the other is used as a labeled antibody labeled with a labeling substance, and sandwich immunoassay is conducted according to the above-described assay method. As a result, an amount of CPA1, an amount of CPA2, an amount of PCPA1, an amount of PCPA2 and a total amount of two or more amounts selected from these amounts (a total amount of CPA1 and PCPA1 (t-CPA1 amount); a total amount of CPA2 and PCPA2 (t-CPA2 amount); a total amount of CPA1, CPA2, PCPA1 and PCPA2 (total t-CPA amount); a total amount of PCPA1 and PCPA2 (total PCPA amount); and a total amount of CPA1 and CPA2 (total CPA amount)) in a sample are assayed on the basis of amounts of labeling substances in one or more complexes selected from thus formed (i) a complex of (a) an immobilized antibody, (b) CPA1 in a sample and (c) a labeled antibody, (ii) a complex of (a) an immobilized antibody, (b) CPA2 in a sample and (c) a labeled antibody, (iii) a complex of (a) an immobilized antibody, (b) PCPA1 in a sample and (c) a labeled antibody, and (iv) a complex of (a) an immobilized antibody, (b) PCPA2 in a sample and (c) a labeled antibody.

In the above assay, any of an antibody (1) and an antibody (2) can be labeled or immobilized. In the case that an antibody (1) and an antibody (2) are antibodies having the same properties (i.e. in the case of using two types of particular antibodies among specific antibodies of the present invention), these antibodies are different in each epitope.

2-3. A CPA/PCPA Binding Antibody

A CPA/PCPA binding antibody used in the above is an antibody having binding property to one or more enzymes selected from CPA1, CPA2, PCPA1 and PCPA2.

Examples of such a CPA/PCPA binding antibody are a specific antibody of the present invention (a t-CPA1 specific antibody, a t-CPA2 specific antibody, a PCPA1 specific antibody and a PCPA2 specific antibody), an antibody which binds to all of CPA1, PCPA1, CPA2 and PCPA2 (hereinafter designates as a t-CPA common antibody) and an antibody which specifically binds to PCPA1 and PCPA2 (hereinafter designates as a PCPA common antibody).

In the above-described CPA/PCPA binding antibody, a t-CPA common antibody is an antibody having reactivity to constant region (t-CPA common region) existing commonly in all of CPA1, CPA2, PCPA1 and PCPA2 (recognizing common region). In other words, said antibody has reactivity to all of CPA1, CPA2, PCPA1 and PCPA2 (recognizing all of CPA1, CPA2, PCPA1 and PCPA2).

A PCPA common antibody is an antibody having reactivity to region commonly existing in pro-region of both PCPA1 and PCPA2 and region not existing in any of CPA1 and CPA2 (PCPA common region)(recognizing common region).

In other words, said antibody has reactivity to each of PCPA1 and PCPA2 (recognizing both PCPA1 and PCPA2), and has substantially no reactivity to CPA1 and CPA2 (not recognizing CPA1 and CPA2).

A specific antibody of the present invention (a t-CPA1 specific antibody, a t-CPA2 specific antibody, a PCPA1 specific antibody and a PCPA2 specific antibody) is described above.

Origin of the above-described t-CPA common antibody and PCPA common antibody (hereinafter sometimes designates as a common antibody of the present invention) is not limited, and can be a polyclonal antibody or a monoclonal antibody. Further a target specific binding molecule prepared by applying evolutionary molecular engineering, for example, a phage antibody and an aptamer having identical properties with a t-CPA common antibody and a PCPA common antibody, can be used in place of the above antibody. These antibodies can optionally be used alone or in combination.

Considering specificity of an antibody having uniform property, a monoclonal antibody is more preferable than a polyclonal antibody.

A method for preparing a t-CPA1 common polyclonal antibody or a PCPA2 common polyclonal antibody includes a conventional method, wherein animals such as equine, bovine, sheep, rabbit, goat, guinea pig, rat, mouse, etc., are immunized by using CPA1, CPA2, PCPA1, PCPA2 or fragments thereof according to a method described in “Introduction to Immunological Experiments” 2nd Ed. Matsuhashi, N. et al. K.K Gakkai Shuppan Center, 1981. Recombinant CPA1, recombinant CPA1, recombinant PCPA1 and recombinant PCPA2 obtained by a known method of genetics can also be used as an immunogen. Further, origin of a recombinant (an immunogen) is not especially limited, and a recombinant derived from human, animals other than human (e.g. monkeys, mice, rats, rabbits, porcine, bovine, sheep, etc.), protista (e.g. nematode) and microorganisms (e.g. yeasts) can be used. Among others, a recombinant of human origin is preferable, and a recombinant of human origin produced from cells of human origin (e.g. human embryonic kidney cells (THE HEK-293), human FL cells, HeLa (human cervical cancer) cells, etc.) is especially preferable.

Further, a method for preparing a t-CPA common monoclonal antibody or a PCPA common monoclonal antibody includes as follows: Using CPA1, CPA2, PCPA1, PCPA2 or fragments thereof as an immunogen, immunized cells such as spleen cells and lymphocytes of animals such as rat and mouse immunized by an immunogen, and cells having permanently proliferating nature such as myeloma cells are fused by known cell fusion technique developed by Koehler and Milstein (Nature, 256, 495, 1975) to prepare a hybridoma, and select a hybridoma which produces a monoclonal antibody specific to assay objects, and a hybridoma is cultured in medium or is administered intraperitoneally in animals to produce an antibody in ascites, then an objective monoclonal antibody is yielded from cultured material or ascites; or cells, which produce an antibody having the above-described properties, are prepared by a known method utilizing gene recombination technique (Eur. J. Immunol., 6, 511, 1976), and cells are cultured to obtain an objective monoclonal antibody.

An antibody can be used in a form of F(ab′)₂, Fab′ or Fab after digesting an antibody by using an enzyme such as pepsin, papain, etc.

As described above, an aptamer can be used in place of an antibody, and such an aptamer can be prepared according to description of U.S. Pat. No. 270,163.

CPA1, CPA2, PCPA1, PCPA2 and fragments thereof used as an immunogen can be obtained from pancreas of human, porcine, bovine, etc. or secretory fluid thereof by known purification methods such as ion-exchange chromatography, hydrophobic interaction chromatography and gel filtration chromatography. Purification of protein can be performed by combining with known chromatographic technique such as affinity chromatography using Sepharose beads coated with a t-CPA1 specific antibody, a t-CPA2 specific antibody, a PCPA1 specific antibody, a PCPA2 specific antibody, a t-CPA common antibody (an antibody binding to both CPA1 and CPA2) or a PCPA common antibody (an antibody binding to both PCPA1 and PCPA2). Recombinant CPA1, recombinant CPA2, recombinant PCPA1 and recombinant PCPA2 obtained by a known method of genetics can also be used as an immunogen. Further, origin of a recombinant (an immunogen) is not especially limited, and a recombinant derived from human, animals other than human (e.g. monkeys, mice, rats, rabbits, porcine, bovine, sheep, etc.), protista (e.g. nematode) and microorganisms (e.g. yeasts) can be used. Among others, a recombinant of human origin is preferable, and a recombinant of human origin produced from cells of human origin (e.g. human embryonic kidney cells (HEK-293), human FL cells, HeLa (human cervical cancer) cells, etc.) is especially preferable.

2-4. A Combination of a Specific Antibody of the Present Invention and a CPA/PCPA Binding Antibody in Sandwich Immunoassay

In Table 2, relationship among combination of an antibody used in the above-described sandwich immunoassay (a combination of an antibody (1): one or more antibodies selected from a specific antibody of the present invention and an antibody (2): one or more antibodies selected from a CPA/PCPA binding antibody (i.e. a specific antibody of the present invention, a t-CPA common antibody and a PCPA common antibody)), a type of formed complex and objects to be directly assayed by it, is shown.

In the case of homogeneous assay, any one of an antibody (1) or an antibody (2) in Table 2 may be labeled, and in the case of heterogeneous assay, either one of an antibody (1) or an antibody (2) may be labeled or immobilized.

In Table 2, a case wherein an antibody (1) and an antibody (2) are an antibody having the same property (i.e. in the case of using two types of specific antibodies in a specific antibody of the present invention), these antibodies are different in their epitope each other.

TABLE 2

| | Combination of | | | |
|---|---|---|---|---|
| | an antibody | | | |
| | Antibody | Antibody | | Object to be |
| | (1) | (2) | Complex to be formed | assayed |
| 1 | t-CPA1 | t-CPA1 | t-CPA1 SpAb/CPA1/t-CPA1 SpAb | Total amount of |
| | SpAb | SpAb | t-CPA1 SpAb/PCPA1/t-CPA1 SpAb | CPA1 and PCPA1 |
| 2 | | t-CPA | t-CPA1 SpAb/CPA1/t-CPA CmAb | (t-CPA1 amount) |
| | | CmAb | t-CPA1 SpAb/PCPA1/t-CPA CmAb | |
| 2 | t-CPA2 | t-CPA2 | t-CPA2 SpAb/CPA2/t-CPA2 SpAb | Total amount of |
| | SpAb | SpAb | t-CPA2 SpAb/PCPA2/t-CPA2 SpAb | CPA2 and PCPA2 |
| | | t-CPA | t-CPA2 SpAb/CPA2/t-CPA CmAb | (t-CPA2 amount) |
| | | CmAb | t-CPA2 SpAb/PCPA2/t-CPA CmAb | |
| 3 | PCPA1 | PCPA1 | PCPA1 SpAb/PCPA1/PCPA1 SpAb | Amount of PCPA1 |
| | SpAb | SpAb | | |
| | | t-CPA1 | PCPA1 SpAb/PCPA1/t-CPA1 SpAb | |
| | | SpAb | | |
| | | PCPA | PCPA1 SpAb/PCPA1/PCPA CmAb | |
| | | CmAb | | |
| | | t-CPA | PCPA1 SpAb/PCPA1/t-CPA CmAb | |
| | | CmAb | | |
| 4 | PCPA2 | PCPA2 | PCPA2 SpAb/PCPA2/PCPA2 SpAb | Amount of PCPA2 |
| | SpAb | SpAb | | |
| | | t-CPA2 | PCPA2 SpAb/PCPA2/t-CPA2 SpAb | |
| | | SpAb | | |
| | | PCPA | PCPA2 SpAb/PCPA2/PCPA CmAb | |
| | | CmAb | | |
| | | t-CPA | PCPA2 SpAb/PCPA2/t-CPA CmAb | |
| | | CmAb | | |
| 5 | t-CPA1 | t-CPA1 | t-CPA1 SpAb/CPA1/t-CPA1 SpAb | Amount of t-CPA1 & |
| | SpAb + t- | SpAb + t- | t-CPA1 SpAb/PCPA1/t-CPA1 SpAb | t-CPA2 |
| | CPA2 | CPA2 | t-CPA2 SpAb/CPA2/t-CPA2 SpAb | Total amount of |
| | SpAb | SpAb | t-CPA2 SpAb/PCPA2/t-CPA2 SpAb | CPA1, CPA2, PCPA1 |
| | | t-CPA | t-CPA1 SpAb/CPA1/t-CPA CmAb | & PCPA2 (Total |
| | | CmAb | t-CPA1 SpAb/PCPA1/t-CPA CmAb | t-CPA amount) |
| | | | t-CPA2 SpAb/CPA2/t-CPA CmAb | |
| | | | t-CPA2 SpAb/PCPA2/t-CPA CmAb | |
| 6 | PCPA1 | PCPA1 | PCPA1 SpAb/PCPA1/PCPA1 SpAb | Amount of PCPA1 & |
| | SpAb + PCPA2 | SpAb + PCPA2 | PCPA2 SpAb/PCPA2/t-CPA2 SpAb | amount of PCPA2 |
| | SpAb | SpAb | | Total amount of |
| | | t-CPA1 | PCPA1 SpAb/PCPA1/t-CPA1 SpAb | PCPA1 & PCPA2 |
| | | SpAb + t- | PCPA2 SpAb/PCPA2/t-CPA2 SpAb | (Total PCPA |
| | | CPA2 | | amount) |
| | | SpAb | | |
| | | PCPA | PCPA1 SpAb/PCPA1/PCPA CmAb | |
| | | CmAb | PCPA2 SpAb/PCPA2/PCPA CmAb | |
| | | t-CPA | PCPA1 SpAb/PCPA1/t-CPA CmAb | |
| | | CmAb | PCPA2 SpAb/PCPA2/t-CPA CmAb | |

Abbreviations:
SpAb: specific antibody
CmAb: common antibody

2-5. An Insoluble Carrier

An insoluble carrier used in the present invention includes any conventionally used carrier, for example, a synthetic polymer such as polystyrene, polypropylene, polyacrylic acid, polymethacrylic acid, polyacrylamide, polyglycidyl methacrylate, polyvinyl chloride, polyethylene, polychlorocarbonate, a silicone resin and silicone rubber, and an inorganic material such as porous glass, ground glass, alumina, silica gel, activated carbon and a metal oxide. These insoluble carriers can be used in various forms such as tubes, beads, discs fragments, micro particles (latex particles) and micro plates. Among others, micro plates, beads and latex particles are especially preferable on the point of washing easiness and operability for simultaneously treating many samples.

A method for immobilizing the above antibody to such an insoluble carrier includes a known immobilizing method, for example, a method for immobilizing by covalent binding or a method for immobilizing by physical adsorption (JP-A-05-41946(1993).

More specifically, a method wherein a solution containing a specific antibody of the present invention, a t-CPA common antibody or a PCPA common antibody, within a range of 1 μg/ml-20 mg/ml, preferably 1 μg/ml -5 mg/ml, and an insoluble carrier are contacted and reacted at proper temperature for specified time to obtain an insoluble carrier (a solid phase) bound with an antibody. In the case of using combination of two or more antibodies, a total amount of antibodies used are designed to be within the above range.

A solvent for preparing a solution containing one or more antibodies selected from a specific antibody of the present invention, a t-CPA common antibody and a PCPA common antibody can be such one as does not inhibit adsorbing or binding of an antibody to an insoluble carrier. For example, a solvent includes purified water phosphate buffer, Tris buffer, preferably Good's buffer, glycine buffer and borate buffer having buffering action at neutral pH, e.g. pH 5.0-10.0, preferably PH 6.5-8.5. Concentration of a buffer agent in the buffer solution is selected from a range from generally 10-500 mM, preferably 10-300 mM. Sugars, salts such as NaCl, surfactants, antiseptics and proteins can be contained in a solution, if amount thereof does not inhibit adsorbing or bonding of an antibody to an insoluble carrier.

Protein blocking performed conventionally in this field, namely treatment wherein an insoluble carrier bound with an antibody, obtained by the method above, is soaked in a solution containing protein, which has no relation to an antibody, for example bovine serum albumin, milk protein such as skim milk and egg albumin, is preferable on the point of preventing non-specific reaction in assaying.

2-6. Labeling

Examples of labeling substances used in the present invention are all labeling substance generally used in this field, for example: enzymes used in enxyme immunoassay (EIA) such as alkaline phosphatase, β-galactosidase, peroxidase, microperoxidase, glucoseoxidase, glucose6-phosphate dehydrogenase, acetylcholine esterase, malic acid dehydrogenase, luciferase, etc.; radioisotopes used in RIA such as ⁹⁹Tc, ¹³¹I, ¹²⁵I, ¹⁴C, ³H, etc.; fluorescent materials used in fluorescence immunoassay (FIA) such as fluorecein, dansyl, fluorescamine, coumarin, naphthylamine fluorecein isothiocyanate, rhodamine, rhodamine-X-isothiocyanate, sulforhodamine 101, lucifer yellow, acridine, acridine isothiocyanate, riboflavine or derivatives thereof, europium (Eu), etc.; luminal derivatives such as isoluminol, luminol, aminoethylisoluminol, aminoethylethylisoluminol, aminopropylisoluminol, aminobutylisoluminol, aminohexylisoluminol, etc.; luminescent materials such as luciferin, bis(2,4,6-trifluorophenyl)oxalate, etc.; ultraviolet absorption materials such as phenol, naphthol, anthracene, or derivatives thereof, etc.; materials having the property of a spin labeling agent represented by oxyl group containing compounds such as 4-amino-2,2,6,6-tetramethylpiperidine-1-oxyl, 3-amino-2,2,5,5-tetramethylpyrrolidine-1-oxyl, 2,6-di-tert-butyl-α-(3,5-di-tert-butyl-4-oxo-2,5-cyclohexadiene-1-ylidene)-p-tolyl oxyl, etc

For binding (labeling) the above exemplified labeling substance to an antibody, a known labeling method generally used in a known EIA, RIA or FIA can be used; for example a method described in “Comprehensive Medical Chemistry Lecture Series” Vol. 8, Yamamura, Y. Ed., 1st Ed., Nakayama Publ. Co. 1971; “Illustrated Fluorescent An antibody Technique” by Kawao, A., 1st Ed. K.K. Soft Science Inc. 1983; and “Enzyme Immunoassay”, Ishikawa, E. et al. Ed., 2nd Ed., Igaku-Shoin, 1982, can be applied. Quite naturally, a conventional labeling method using a reaction with avidin (or streptavidin) and biotin.

In the case of applying a reaction with avidin (or streptavidin) and biotin in a method for bonding biotin to an antibody, various methods can be included such as a commercially available biotinylated reagent, more specifically, succinimide introduced biotin (e.g. NHS-biotin) or a compound bound with N-hydroxysuccinimide (NHS) and biotin through a spacer reacted with amino group in an antibody or an antigen protein (e.g. J. Biol. Chem. 254, 272-279, 1989, etc.); commercially available N-[6-(biotinamide)hexyl]-3′-(2′-pyridyldithio)propionamide (biotin-HPDP) or N-iodoacetyl-N-biotinylhexylenediamin is reacted with a thiol group in an antibody or an antigen (e.g. Ann. N.Y. Acad. Sci., 254, 203, 1975, etc.); and hydrazino group introduced biotin is reacted with an aldehyde group of an antigen or an antibody, which is modified to have aldehyde groups (e.g. J. Biol. Chem., 172, 71, 1948; Biotech. Appl. Biochem., 9, 488-496, 1987, etc.).

A modified level of an antigen or an antibody by biotin is 0.2-10 times molar ratio for an antigen or an antibody, preferably 1-5 times molar ratio. Since in the case of higher modification quantity with biotin, there may be problems of insolubility of an antigen or an antibody is decreased or an antigen-antibody reaction is inhibited, while in the case of lower modification quantity with biotin, there may be a problem that sensitivity does not achieve initial goal, caution is necessary.

Commercially available enzyme labeled avidin or streptavidin can be used as it is, and quality and purification accuracy are not especially limited. Amount of use depends on amount ratio of a bitin modified antigen (or an antibody) to objects to be assayed or items to be assayed and is not especially limited. Generally, concentration in a reaction mixture is selected in a range 0.01-5,000 μg/l, preferably 0.1-1,000 μg/l, more preferably 5-1,000 μg/l.

A stabilizing agent conventionally used in this field such as sugars, proteins, surfactants, and the like can be contained in a solution within concentration used generally in this field.

A method for measuring amount of a label in an antigen-antibody complex generated by an antigen-antibody reaction is different depending on labeling substance type, and measurement can be performed according to a routine method depending on detectable nature of labeling substance by any methods. In the case that a labeling substance is an enzyme, a conventional method of EIA, for example by a method described in “Enzyme immunoassay” (Proteins, Nucleic Acids and Enzymes, Suppl. No 31, Kitagawa, T. et al. Ed., p. 51-63, Kyoritsu Publ. Co., 1987) can be applied. In the case that a labeling substance is a radioisotope, measurement can be performed according to a conventional method of RIA. Namely, a measurement apparatus such as immersion GM counter, a liquid scintillation counter, a well-type scintillation counter, a counter for HPLC, and the like is selectively used depending on type and intensity of radiation emitted by a radioisotope (e.g. “Comprehensive Medical Chemistry Lecture Series” Vol. 8, Yamamura, Y. Ed., 1st Ed., Nakayama Publ. Co. 1971). In the case that a labeling substance is a fluorescent substance, conventional means of FIA using measurement apparatus such as a fluorometer is used according to description in “Illustrated Fluorescent An antibody Technique” by Kawao, A., 1st Ed. K.K. Soft Science Inc. 1983. In the case that the labeling substance is a luminous substance, conventional means using measurement apparatus such as a photocounter (Proteins, Nucleic Acids and Enzymes, Suppl. No 31, Kitagawa, T. et al. Ed., p. 252-263, Kyoritsu Publ. Co., 1987). In the case that a labeling substance is a substance having UV absorption, measurement can be performed by conventional means using measurement apparatus such as a spectrophotometer. In the case that a labeling substance has spin nature, conventional means using measurement apparatus such as electron spin resonance apparatus can be used (“Enzyme immunoassay” (Proteins, Nucleic Acids and Enzymes, Suppl. No 31, Kitagawa, T. et al. Ed., p. 264-271, Kyoritsu Publ. Co., 1987).

In the case that a labeling substance is an enzyme, assay can be performed by a conventional method by reacting a enzyme with a coloring agent to induce a coloring reaction, and measuring a generated amount of a pigment by using a spectrophotometer.

A coloring agent used for such purpose includes one conventionally used in this field such as tetramethylbenzidine, o-phenylenediamie, o-nitrophenyl-galactoside, 2,2′-azinobis(3-ethylbenzothiazoline-6-sulfonicacid) (ABTS), N-ethyl-N-sulfopropyl-m-anisidine(ADPS), p-nitrophenyl phosphate, etc.

For terminating a coloring reaction, a reaction termination method conventionally used in this field can be applied, for example by adding a enzyme inhibitor such as 1-6 N sulfuric acid into a reaction mixture.

2-7. A Separation Improving Substance

In an assay method applying HPLC, to separate clearly an antigen-antibody complex and a free labeled antibody (a labeled CPA1 specific antibody, a labeled CPA2 specific antibody, a labeled PCPA1 specific antibody, a labeled PCPA2 specific antibody, a labeled t-CPA common antibody or a labeled PCPA common antibody), a substance for improving separation of the complex and the free labeled antibody (hereinafter designates as “a separation improving substance”) disclosed in JP-A-07-191027, JP-A-09-901995, WO 02/082083, and the like may optionally be bound to a specific antibody of the present invention (a CPA1 specific antibody, a CPA2 specific antibody, a PCPA1 specific antibody or a PCPA2 specific antibody), a t-CPA common antibody or a PCPA common antibody.

A separation improving substance used for such an object is preferably, for example, nucleic acids such as DNA, RNA, etc.; proteins such as α-chymotrypsinogen, β-galactosidase, lysozyme, cytochrome C, trypsin inhibitor, etc.; peptide containing an amino acid such as phenylalanine, proline, arginine, lysine, aspartic acid, glutamic acid, etc.; halogen atoms such as bromine atom, chlorine atom, iodine atom, etc.; synthetic polymers such as polyethylene glycol,etc.; polyamino acids such as polyglutamic acid, polyaspartic acid, polylysine, polyarginine, polyphenylalanine, polytyrosine, etc.; alkyl chains having 3 to 10 carbon atoms; fatty acids such as palmitic acid, oleic acid, stearic acid, etc.; chemical substances containing a reactive group which enables to bind with protein or protein binding specific sugar chain and having hydrophobic or ionic nature such as N-(ε-maleimidocaproyloxy)succinimide (EMCS), N-succinimidyl-6-maleimidohexanoate, bismaleimido hexane (BMH), octylamine, etc.; peptides containing strong acid residue group such as 4-(p-maleimidophenyl)butyrylAla-(Tyr (SO₃H))₅, 4-(p-maleimidophenyl)butyrylAla-(Tyr(SO₃H))₈, etc disclosed in JP-A-9-301995. A separation improving substance may be selected, as appropriate, considering properties (for example, pH stability, hydrophobicity, solubility in an aqueous solution, isoelectric point, and the like) of a specific antibody of the present invention (a CPA1 specific antibody, a CPA2 specific antibody, a PCPA1 specific antibody or a PCPA2 specific antibody), a t-CPA common antibody or a PCPA common antibody to be used.

Binding of a separation improving substance and the specific antibody of the present invention (a CPA1 specific antibody, a CPA2 specific antibody, a PCPA1 specific antibody or a PCPA2 specific antibody), a t-CPA common antibody or a PCPA common antibody may be performed according to (1) a known binding method for labeling substance and an antibody generally practiced in known EIA (ELISA), RIA, FIA, and the like (for example, Medical Experiment Course, vol. 8, under the supervision of Y. Yamamura, 1st ed., Nakayama Shoten 1971; Illustration Fluorescent Antibody, A. Kawasho, 1st ed., Soft Science Inc., 1983; Method for Measuring Enzyme Immunity, edited by E. Ishikawa, T. Kawaiand K. Miyai, 2nd ed. Igaku Shoin, 1982, etc.) and (2) a known modification and binding method of substances (for example, Chemical Modification of Protein, vol. 1 and vol. 2, edited by I. Uritani, K. Shimura, M. Nakamura and M. Funatsu, 1st ed., Gakkai Shuppan center Inc., 1981; Polyethylene glycol-modified protein, Y. Inada et al., Biochemistry, vol. 62, No. 11, P1351-1362, The Japanese Biochemistry Society, 1990; DNA PROBES, George H. K. and Mark M. M., STOCKTON PRESS, 1989, W002/082083 official gazette, etc.).

2-8. Latex Nephelometry

One example of a homogeneous measuring method other than the above is a method (1)conducting the above described latex nephelometry by using an immobilized antibody in which one or more kinds of antibodies selected from specific antibodies of the present invention are bound to an insoluble carrier such as a latex particle, and measuring degree (level) of agglomeration formed derived from thus formed one or more kinds of a complex selected from (i) a complex of (a) an immobilized antibody and (b) CPA1 in a sample, (ii) a complex of (a) an immobilized antibody and (b) CPA2 in a sample, (iii) a complex of (a) an immobilized antibody and (b) PCPA1 in a sample and (iv) a complex of (a) the immobilized antibody and (b) PCPA2 in a sample, based on change in scattered light, transmitted light, and the like, to determine amounts of CPA1, CPA2, PCPA1, PCPA2 and a total amount of two or more kinds of amount selected from the above amounts [a total amount of CPA1 and PCPA1 (t-CPA1 amount), a total amount of CPA2 and PCPA2 (t-CPA2 amount), a total amount of CPA1, CPA2, PCPA1 and PCPA2 (total t-CPA amount), a total amount of PCPA1 and PCPA2 (total PCPA amount) and a total amount of CPA1 and CPA2 (total CPA amount)].

In the mentioned above, a specific antibody relevant to the present invention to be used is preferably a polyclonal antibody. It is also possible to use in combination with two or more kinds of specific antibodies relevant to the present invention. The combination is similar to a combination of the specific antibody of the present invention and a CPA/PCPA binding antibody in the above sandwich method. However, when two or more kinds of a specific antibody of the present invention having the same property are used (that is, when the two particular ones among specific antibodies of the present invention are used), these antibodies have a different epitope from each other.

While any latex generally used in this field may be used as the above latex particle without special limitation, styrene-based latex such as polystyrene latex and acrylic acid-based latex are preferably used. Among these latex particles, polystyrene latex particles, and the like obtained by an emulsion polymerization without using an emulsifier are particularly preferable, because they have smooth adsorbability of protein or peptide due to strong hydrophobicity of the surface thereof and disperse stably in a solution even without an emulsifier due to repulsion of negative charges at the surface thereof. Various kinds of modified latex (for example, carboxylic acid-modified latex obtained by introducing carboxyl groups into the above polystyrene), magnetic latex (latex incorporated with magnetic particles), and the like can be used, if necessary. Commercial latex particles may also be used.

Average diameter of latex particles is not especially limited as long as it is a size generally used in this field, however, latex with relatively small average diameter (that is, latex having large surface area per unit weight) is preferable, because it can effectively sensitize an antibody. Typically, average diameter is usually 0.05 to 2.4 μm, preferably 0.05 to 1.0 μm and more preferably 0.05 to 0.28 μm. A combined use may be possible of two kinds of latex particles with different average diameter selected from those in the above ranges. A preferable diameter combination is, for example, the one usually in 0.05 to 0.3 μm, preferably 0.05 to 0.18 μm and the other usually in 0.18 to 0.5 μm, preferably in 0.18 to 0.28 μm. Diameter difference between two kinds of latex particles with different average diameter is preferably at least 0.05 nm or more.

An antibody may be immobilized at a latex particle according to a known method, for example, a method comprising suspending an antibody and a latex particle in, for example, a buffer solution, reacting them at 20 to 30° C. for 2 to 3 hours and then subjecting the product to post-treatment such as centrifugal separation and blocking treatment, generally practiced in this field. Use amount of an antibody here may be an amount generally used in this field and the same use amount as in the above described method for immobilizing an antibody at an insoluble carrier.

In the above method, scattered light or transmitted light may be measured by using general-purpose instrument for biochemistry such as an automatic analyzer and a spectrophotometer, as well as instrument exclusive for nephelometry such as a laser nephelometer, according to a manual of each instrument for detail.

2-9. An Electrophoretic Method

One example of a heterogeneous measuring method other than the above is an electrophoretic method that makes use of, for example, an antigen-antibody reaction.

In other words, the method comprises performing an electrophoretic method such as the above described capillary (chip) electrophoretic method using one or more kinds of an antibody selected from a specific antibody of the present invention, forming one or more kinds of a complex selected from (i) a complex of (a) an antibody and (b) CPA1 in a sample, (ii) a complex of (a) an antibody and (b) CPA2 in a sample, (iii) a complex of (a) an antibody and (b) PCPA1 in a sample and (iv) a complex of (a) an antibody and (b) PCPA2 in a sample, separating said complex from a free antibody that is not involved in said complex forming, using an electrophoretic method, if necessary, in the presence of a charged polymer to avoid serum effect and thus determining, based on an amount of a separated complex, the amounts of CPA1, CPA2, PCPA1, PCPA2 or total amount of two or more kinds of an amount selected from the above amounts [a total amount of CPA1 and PCPA1 (t-CPA1 amount), a total amount of CPA2 and PCPA2 (t-CPA2 amount), a total amount of CPA1, CPA2, PCPA1 and PCPA2 (total t-CPA amount), a total amount of PCPA1 and PCPA2 (total PCPA amount) and a total amount of CPA1 and CPA2 (total CPA amount)] in the sample.

In the above method, at least one kind from one or more kinds of antibodies may bind to a substance (a separation improving substance) that can change separation characteristics of an object to be assayed by forming a complex of an object to be assayed, an antibody and a separation improving substance, and binding with an object to be assayed through an antibody, to form a conjugate of an antibody and a separation improving substance. When two or more kinds of antibodies are used, or two or more kinds of conjugates are used, or one or more kinds of antibody and conjugate are used in combination, these antibodies (including antibodies in conjugates) have different epitope from each other.

As at least one kind of antibody or conjugate (antibody or separation improving substance, constituting a conjugate) is usually labeled by a labeling substance in the above method, a labeling substance in a complex or a free labeling substance is measured and an amount of an object to be assayed in a sample is determined based on the above measurement.

A monoclonal antibody is preferable for a specific antibody of the present invention to be used above. It is also possible to use in combination with two or more kinds of a specific antibody of the present invention. The combination is similar o a combination of the specific antibody of the present invention and a CPA/PCPA binding antibody in the above sandwich method. However, when two or more kinds of a specific antibody of the present invention having the same property, are used (that is, when two particular kinds among specific antibodies of the present invention are used), these antibodies have different epitope from each other.

A method for separating a complex from a free antibody (labeled antibody, labeled conjugate) that is not involved in complex formation preferably includes, an electrical separation method such as an isoelectric focusing, an SDS-polyacrylamide electrophoresis, an agarose gel electrophoresis, an acrylamide electrophoresis and a dielectrophoresis, more preferably a capillary (chip) electrophoresis and a dielectrophoresis and still more preferably a capillary chip electrophoresis.

A capillary electrophoresis using micro fluidic device equipped with at least one separation channel having inner diameter of 0.1 to 500 μm is particularly preferable. Such a micro fluidic device includes, for example, a device (chip) described in W002/082083 official gazette, Published Japanese translation of PCT international publication for patent application No. 2000-513813 official gazette, Published Japanese translation of PCT international publication for patent application No. 2000-515630 official gazette, Published Japanese translation of PCT international publication for patent application No. 2002-516343 official gazette, Published Japanese translation of PCT international publication for patent application No. 2001-517794 official gazette, Published Japanese translation of PCT international publication for patent application No. 2001-521622 official gazette, Published Japanese translation of PCT international publication for patent application No. 2002-514300 official gazette, and the like, wherein materials and preparation methods of these devices are also described. Reagents (for example, a polymer having molecular sieve effect and a buffer solution) generally used in this field can also be used for said device (chip).

A charged polymer to be used above includes polyanionic polymers such as poly-dIdC, heparin sulfate, dextran sulfate, poly-tungstic acid, poly-anisole sulfate, polyvinyl sulfate, poly(acrylic acid), chondroitin sulfuric acid, DNA, etc.; polycationic polymers such as poly(allylamine), poly-lysine, poly-histidine, chitosan, protamine, polyethyleneimine, poly-arginine, etc., preferably polyanionic polymers, more preferably heparin sulfate.

A separation improving substance to be used above is a charged molecule, that is, an anionic molecule or a cationic molecule. Specifically, it is a substance having such a property among separation improving substances described above and preferably a nucleotide chain and sulfonated polypeptide, and more preferably a DNA chain. A method for binding said separation improving substances and an antibody is as described above.

A labeling substance includes a fluorescent pigment that can bind to a nucleic acid chain such as an intercalating pigment described in W002/082083 official gazette, and the like, in addition to the above-described labeling substance and a method for binding (labeling) these labeling substances to an antibody or a separation improving substance as described above.

A method for measuring a labeling substance in a separated complex or a free labeling substance is also the same as a method for measuring an amount of a label in an antigen-antibody complex generated by the above antigen-antibody reaction.

2-10. A Typical Assay Method

in a method for immunologically assaying of the present invention, among sandwich immunoassays using an insoluble carrier, a coloring method for various objects to be assayed in a sample is exemplified below.

(1) Assay of a Total Amount of CPA1 and PCPA1 (t-CPA1 Amount)

A sample is contacted with a solid phase, which is an insoluble carrier immobilized with a t-CPA1 specific antibody (the primary antibody) of the present invention, and reacted at 4-40° C. for 3 minutes-16 hours to generate an antigen-antibody complex (a complex-i: two types of complexes including an immobilized t-CPA1 specific antibody/CPA1 complex, and an immobilized t-CPA1 specific antibody/PCPA1 complex) on the insoluble carrier. Subsequently, the complex is reacted with a labeled t-CPA1 specific antibody bound with a labeling substance (the secondary antibody: proviso that epitope is different from the primary antibody) at 4-40° C. for 3 minutes-16 hours to generate a labeled antigen-antibody complex (a complex-ii: two types of complexes including an immobilized t-CPA1 specific antibody/CPA1/a labeled t-CPA1 specific antibody complex, and an immobilized t-CPA1 specific antibody/PCPA1/a labeled t-CPA1 specific antibody complex) on the insoluble carrier, then an amount of the labeling substance in the complex-ii is assayed. Thus obtained amount of labeling substance is applied to a calibration curve showing relationship between an amount of a labeling substance and concentration of CPA1 and PCPA1 (t-CPA1 concentration), which is obtained by performing the same operation by using the same reagent as above on a solution containing previously known concentration of CPA1 and PCPA1 (a t-CPA1 solution), to obtain a total amount of CPA1 and PCPA1 (t-CPA1 amount) (concentration) in the sample. A total amount of CPA1 and PCPA1 (t-CPA1 amount) can also be obtained as relative value based on a proper standard (e.g. pooled serum of healthy subjects) as an amount of 100 AU (arbitrary units)

In the case that a labeled antibody (the secondary antibody) is a biotin labeled antibody, a solid phase, which is an insoluble carrier immobilized with the primary antibody, is reacted with a sample, and a biotin labeled antibody is reacted therewith to generate a biotin labeled antigen-antibody complex consisting of a biotin labeled antibody and an antigen, subsequently it is reacted with enzyme labeled streptavidin, which is labeled with an enzyme such as peroxidase, and a enzyme labeled streptavidin-biotin labeled antigen-antibody complex is generated on a solid phase. Then a substrate for the labeled enzyme is reacted therewith and an amount of the labeled enzyme in the enzyme labeled streptavidin-the biotin labeled antigen-antibody complex on the solid phase is assayed. Subsequently, a total amount of CPA1 and PCPA1 (t-CPA1 amount) in the sample can be determined from a calibration curve by the same method as described above.

A typical example of immunoassay of a total amount of CPA1 and PCPA1 (t-CPA1 amount) of the present invention is outlined below by exemplifying a method for assaying a total amount of CPA1 and PCPA1 (t-CPA1 amount) in a sample by using the peroxidase (POD) labeled secondary antibody as a labeled antibody in a coloring method.

A sample is contacted with a solid phase, which is an insoluble carrier immobilized with a t-CPA1 specific antibody (the primary antibody) of the present invention to generate an antigen-antibody complex (a complex-i: two types of complexes including an immobilized t-CPA1 specific antibody/CPA1 complex, and an immobilized t-CPA1 specific antibody/PCPA1 complex) on an insoluble carrier. Subsequently, the complex is reacted with a POD labeled t-CPA1 specific antibody (the secondary antibody) to generate a POD labeled antigen-antibody complex (a complex-ii: two types of complexes including an immobilized t-CPA1 specific antibody/CPA1/a POD labeled t-CPA1 specific antibody complex, and an immobilized t-CPA1 specific antibody/PCPA1/a POD labeled t-CPA1 specific antibody complex) on the insoluble carrier. Subsequently, an ODP (o-phenylenediamine dihydrochloride) solution (a coloring substance) and a H₂O₂ solution are added. After definite time from reaction start, 2N sulfuric acid is added to terminate the reaction, and then an amount of the labeling substance in the labeled antigen-antibody complex (complex-ii) on the insoluble carrier is assayed as coloring intensity (absorbancy). Thus obtained coloring intensity (absorbancy) is applied to a calibration curve showing relationship between coloring intensity (absorbancy) and an amount of CPA1 and PCPA1 (t-CPA1 amount), which is obtained by performing the same operation by using the same reagent as above on a solution containing previously known concentration of CPA1 and PCPA1 (t-CPA1 solution), to obtain a total amount of CPA1 and PCPA1 (t-CPA1 amount) in the sample.

As described above, in the above method, although assay can be performed in the same manner by using a t-CPA common antibody in place of a t-CPA1 specific antibody used as the primary antibody (refer to Table 2), a t-CPA1 specific antibody which has different epitope from the primary antibody is preferable.

Further, the same assay can be performed by exchanging the primary antibody and the secondary antibody, however, use of a t-CPA1 specific antibody as the primary antibody and a t-CPA1 specific antibody as the secondary antibody is preferable due to forming order of an antigen-antibody complex.

(2) Assay of a Total Amount of CPA2 and PCPA2 (t-CPA2 Amount)

A sample is contacted with a solid phase which is an insoluble carrier immobilized with a t-CPA2 specific antibody (the primary antibody) of the present invention, and reacted at 4-40° C. for 3 minutes-16 hours to generate an antigen-antibody complex (a complex-i: two types of complexes including an immobilized t-CPA2 specific antibody/CPA2 complex, and an immobilized t-CPA2 specific antibody/PCPA2 complex) on the insoluble carrier. Subsequently, the complex is reacted with a labeled t-CPA2 specific antibody bound with a labeling substance (the secondary antibody: proviso that epitope is different from the primary antibody) at 4-40° C. for 3 minutes-16 hours to generate a labeled antigen-antibody complex (a complex-ii: two types of complexes including an immobilized t-CPA2 specific antibody/CPA2/a labeled t-CPA2 specific antibody complex, and an immobilized t-CPA2 specific antibody/PCPA2/a labeled t-CPA2 specific antibody complex) on the insoluble carrier, then an amount of the labeling substance in the complex-ii is assayed. Thus obtained amount of labeling substance is applied to a calibration curve showing relationship between an amount of labeling substance and concentration of CPA2 and PCPA2 (t-CPA2 concentration), which is obtained by performing the same operation by using the same reagent as above on a solution containing previously known concentration of CPA2 and PCPA2 (t-CPA2 solution), to obtain a total amount of CPA2 and PCPA2 (t-CPA2 amount) (concentration) in the sample. Total amount of CPA2 and PCPA2 (t-CPA2 amount) can also be obtained as relative value based on a proper standard (e.g. pooled serum of healthy subjects) as an amount of 100 AU (arbitrary units).

In the case that a labeled antibody (the secondary antibody) is a biotin labeled antibody, a solid phase, which is an insoluble carrier immobilized with the primary antibody, is reacted with a sample, and a biotin labeled antibody is reacted therewith to generate a biotin labeled antigen-antibody complex consisting of a biotin labeled antibody and an antigen, subsequently it is reacted with enzyme labeled streptavidin, which is labeled with an enzyme such as peroxidase, and an enzyme labeled streptavidin-a biotin labeled antigen-antibody complex is generated on the solid phase. Then a substrate for the labeled enzyme is reacted therewith and an amount of a labeled enzyme in the enzyme labeled streptavidin-the biotin labeled antigen-antibody complex on the solid phase is assayed. Subsequently, a total amount of CPA2 and PCPA2 (t-CPA2 amount) in the sample can be determined from a calibration curve by the same method as described above.

A typical example of immunoassay of a total amount of CPA2 and PCPA2 (t-CPA2 amount) of the present invention is outlined below by exemplifying a method for assaying a total amount of CPA2 and PCPA2 (t-CPA2 amount) in a sample by using the peroxidase (POD) labeled secondary antibody as a labeled antibody in a coloring method.

A sample is contacted with a solid phase, which is an insoluble carrier immobilized with a t-CPA2 specific antibody (the primary antibody) of the present invention, to generate an antigen-antibody complex (a complex-i: two types of complexes including an immobilized t-CPA2 specific antibody/CPA2 complex, and an immobilized t-CPA2 specific antibody/PCPA2 complex) on the insoluble carrier. Subsequently, the complex is reacted with a POD labeled t-CPA2 specific antibody (the secondary antibody) to generate a POD labeled antigen-antibody complex (a complex-ii: two types of complexes including an immobilized t-CPA2 specific antibody/CPA2/a POD labeled t-CPA2 specific antibody complex, and an immobilized t-CPA2 specific antibody/PCPA2/a POD labeled t-CPA2 specific antibody complex) on the insoluble carrier. Subsequently, an ODP (o-phenylenediamine dihydrochloride) solution (a coloring substance) and a H₂O₂ solution are added. After definite time from reaction start, 2N sulfuric acid is added to terminate the reaction, and then an amount of the labeling substance in the labeled antigen-antibody complex (complex-ii) on the insoluble carrier is assayed as coloring intensity (absorbancy). Thus obtained coloring intensity (absorbancy) is applied to a calibration curve showing relationship between coloring intensity (absorbancy) and an amount of CPA2 and PCPA2 (t-CPA2 amount), which is obtained by performing the same operation by using the same reagent as above on a solution containing previously known concentration of CPA2 and PCPA2 (t-CPA2 solution), to obtain a total amount of CPA2 and PCPA2 (t-CPA2 amount) in the sample.

As described above, in the above method, although assay can be performed in the same manner by using a t-CPA common antibody in place of a t-CPA2 specific antibody used as the primary antibody (refer to Table 2), a t-CPA2 specific antibody which has different epitope from the primary antibody is preferable.

Further, the same assay can be performed by exchanging the primary antibody and the secondary antibody, however, use of a t-CPA2 specific antibody as the primary antibody and a t-CPA2 specific antibody as the secondary antibody is preferable due to forming order of an antigen-antibody complex.

(3) Assay of an Amount of PCPA1

A sample is contacted with a solid phase, which is an insoluble carrier immobilized with a t-CPA1 specific antibody (the primary antibody) of the present invention, and reacted at 4-40° C. for 3 minutes-16 hours to generate an antigen-antibody complex (a complex-i: an immobilized t-CPA1 specific antibody/PCPA1 complex) on the insoluble carrier. Subsequently, the complex is reacted with a labeled PCPA1 specific antibody bound with a labeling substance at 4-40° C. for 3 minutes-16 hours to generate a labeled antigen-antibody complex (a complex-ii: an immobilized t-CPA1 specific antibody/PCPA1/a labeled PCPA1 specific antibody complex) on the insoluble carrier, then an amount of the labeling substance in the complex-ii is assayed. Thus obtained amount of the labeling substance is applied to a calibration curve showing relationship between an amount of labeling substance and concentration of PCPA1, which is obtained by performing the same operation by using the same reagent as above on a solution containing previously known concentration of PCPA1, to obtain an amount of PCPA1 (concentration) in the sample. The amount of PCPA1 (concentration) may also be obtained as relative value based on a proper standard (e.g. pooled serum of healthy subjects) as an amount of 100 AU (arbitrary units).

In the case that a labeled antibody (the secondary antibody) is a biotin labeled antibody, a solid phase, which is an insoluble carrier immobilized with the primary antibody, is reacted with a sample, and a biotin labeled antibody is reacted therewith to generate a biotin labeled antigen-antibody complex consisting of a biotin labeled antibody and an antigen, subsequently it is reacted with enzyme labeled streptavidin, which is labeled with an enzyme such as peroxidase, and an enzyme labeled streptavidin-a biotin labeled antigen-antibody complex is generated on the solid phase. Then a substrate for the labeled enzyme is reacted therewith and an amount of the labeled enzyme in the enzyme labeled streptavidin-biotin labeled antigen-antibody complex on the solid phase is assayed. Subsequently, an amount of PCPA1 in the sample can be determined from a calibration curve by the same method as described above.

A typical example of immunoassay of an amount of PCPA1 of the present invention is outlined below by exemplifying a method for assaying an amount of PCPA1 in a sample by using a peroxidase (POD) labeled secondary antibody as a labeled antibody in a coloring method.

A sample is contacted with a solid phase, which is an insoluble carrier immobilized with a t-CPA1 specific antibody (the primary antibody) of the present invention, to generate an antigen-antibody complex (a complex-i: an immobilized t-CPA1 specific antibody/PCPA1 complex) on the insoluble carrier. Subsequently, the complex is reacted with a POD labeled PCPA1 specific antibody (the secondary antibody) to generate a POD labeled antigen-antibody complex (a complex-ii: an immobilized t-CPA1 specific antibody/PCPA1/a POD labeled PCPA1 specific antibody complex) on the insoluble carrier. An ODP (o-phenylenediamine dihydrochloride) solution (a coloring substance) and a H₂O₂ solution are added. After definite time from reaction start, 2N sulfuric acid is added to terminate the reaction, and then an amount of the labeling substance in the labeled antigen-antibody complex (complex-ii) on the insoluble carrier is assayed as coloring intensity (absorbency). Thus obtained coloring intensity (absorbency) is applied to a calibration curve showing relationship between coloring intensity (absorbancy) and an amount of PCPA1, which is obtained by performing the same operation by using the same reagent as above on a solution containing previously known concentration of PCPA1, to obtain an amount of PCPA1 in the sample.

As described above, in the above method, although assay can be performed in the same manner by using a PCPA1 specific antibody (proviso that epitope is different from the secondary antibody), a PCPA common antibody or a t-CPA common antibody in place of a t-CPA1 specific antibody used as the primary antibody (refer to Table 2), a t-CPA1 specific antibody is preferable.

Further, the same assay can be performed by exchanging the primary antibody and the secondary antibody, however, use of a PCPA1 specific antibody as the secondary antibody is preferable, especially use of a t-CPA1 specific antibody as the primary antibody and a PCPA1 specific antibody as the secondary antibody is preferable due to forming order of an antigen-antibody complex.

(4) Assay of an Amount of PCPA2

A sample is contacted with a solid phase, which is an insoluble carrier immobilized with a t-CPA2 specific antibody (the primary antibody) of the present invention, and reacted at 4-40° C. for 3 minutes-16 hours to generate an antigen-antibody complex (a complex-i: an immobilized t-CPA2 specific antibody/PCPA2 complex) on the insoluble carrier. Subsequently, the complex is reacted with a labeled PCPA2 specific antibody bound with a labeling substance at 4-40° C. for 3 minutes-16 hours to generate a labeled antigen-antibody complex (a complex-ii: an immobilized t-CPA2 specific antibody/PCPA2/a labeled PCPA2 specific antibody complex) on the insoluble carrier, then an amount of the labeling substance in the complex-ii is assayed. Thus obtained amount of the labeling substance is applied to a calibration curve showing relationship between an amount of labeling substance and concentration of PCPA2, which is obtained by performing the same operation by using the same reagent as above on a solution containing previously known concentration of PCPA2, to obtain an amount of PCPA2 (concentration) in the sample. The amount of PCPA2 (concentration) may also be obtained as relative value based on a proper standard (e.g. pooled serum of healthy subjects) as an amount of 100 AU (arbitrary units).

In the case that a labeled antibody (the secondary antibody) is a biotin labeled antibody, a solid phase, which is an insoluble carrier immobilized with the primary antibody, is reacted with a sample, and a biotin labeled antibody is reacted therewith to generate a biotin labeled antigen-antibody complex consisting of a biotin labeled antibody and an antigen, subsequently it is reacted with enzyme labeled streptavidin, which is labeled with an enzyme such as peroxidase, and an enzyme labeled streptavidin-a biotin labeled antigen-antibody complex is generated on the solid phase. Then a substrate for the labeled enzyme is reacted therewith and an amount of the labeled enzyme in the enzyme labeled streptavidin-a biotin labeled antigen-antibody complex on the solid phase is assayed. Subsequently, an amount of PCPA2 in the sample can be determined from a calibration curve by the same method as described above.

A specific example of immunoassay of an amount of PCPA2 of the present invention is outlined below by exemplifying a method for assaying an amount of PCPA2 in a sample by using the peroxidase (POD) labeled secondary antibody as a labeled antibody in a coloring method.

A sample is contacted with a solid phase, which is an insoluble carrier immobilized with a t-CPA2 specific antibody (the primary antibody) of the present invention, to generate an antigen-antibody complex (a complex-i: an immobilized t-CPA2 specific antibody/PCPA2 complex) on the insoluble carrier. Subsequently, the complex is reacted with a POD labeled PCPA2 specific antibody (the secondary antibody) to generate a POD labeled antigen-antibody complex (a complex-ii: an immobilized t-CPA2 specific antibody/PCPA2/a POD labeled PCPA2 specific antibody complex) on the insoluble carrier. An ODP (o-phenylenediamine dihydrochloride) solution (a coloring substance) and a H₂O₂ solution are added. After definite time from reaction start, 2N sulfuric acid is added to terminate the reaction, and then an amount of the labeling substance in the labeled antigen-antibody complex (complex-ii) on the insoluble carrier is assayed as coloring intensity (absorbency). Thus obtained coloring intensity (absorbancy) is applied to a calibration curve showing relationship between coloring intensity (absorbancy) and an amount of PCPA2, which is obtained by performing the same operation by using the same reagent as above on solution containing previously known concentration of PCPA2, to obtain an amount of PCPA2 in the sample.

As described above, in the above method, although assay can be performed in the same manner by using a PCPA2 specific antibody (proviso that epitope is different from the secondary antibody), a PCPA common antibody or a t-CPA common antibody in place of a t-CPA2 specific antibody used as the primary antibody (refer to Table 2), a t-CPA2 specific antibody is preferable.

Further, the same assay can be performed by exchanging the primary antibody and the secondary antibody, however, use of a PCPA2 specific antibody as the secondary antibody is preferable, especially use of a t-CPA2 specific antibody as the primary antibody and a PCPA2 specific antibody as the secondary antibody is preferable due to forming order of an antigen-antibody complex.

(5) Assay of a Total Amount of CPA1, CPA2, PCPA1 and PCPA2 (Total t-CPA Amount)

Although a total t-CPA amount can be obtained (calculated) by summing up a total amount of CPA1 and PCPA1 (t-CPA amount) obtained in the above (1) and a total amount of CPA2 and PCPA2 (t-CPA2 amount) obtained in the above (2), a total t-CPA amount can be obtained by one time assay accrding to the following method.

A sample is contacted with a solid phase, which is an insoluble carrier immobilized with a t-CPA1 specific antibody and a t-CPA2 specific antibody (the primary antibody) of the present invention, and reacted at 4-40° C. for 3 minutes-16 hours to generate an antigen-antibody complex (a complex-i: four types of complexes including an immobilized t-CPA1 specific antibody/CPA1 complex, an immobilized t-CPA1 specific antibody/PCPA1 complex, an immobilized t-CPA2 specific antibody/CPA2 complex, and an immobilized t-CPA2 specific antibody/PCPA2 complex) on the insoluble carrier. Subsequently, the complex is reacted with a labeled t-CPA1 specific antibody and the labeled t-CPA2 specific antibody bonded with a labeling substance (the secondary antibody: proviso that epitope is different from the primary antibody) at 4-40° C. for 3 minutes-16 hours to generate a labeled antigen-antibody complex (a complex-ii: four types of complexes including an immobilized t-CPA1 specific antibody/CPA1/a labeled t-CPA1 specific antibody complex, an immobilized t-CPA1 specific antibody/PCPA1/a labeled t-CPA1 specific antibody complex, an immobilized t-CPA2 specific antibody/CPA2/a labeled t-CPA2 specific antibody complex, and an immobilized t-CPA2 specific antibody/PCPA2/a labeled t-CPA2 specific antibody complex) on the insoluble carrier, then an amount of the labeling substance in the complex-ii is assayed. Thus obtained amount of labeling substance is applied to a calibration curve showing relationship between an amount of a labeling substance and concentrations of CPA1, PCPA1, CPA2 and PCPA2 (total t-CPA concentration), which is obtained by performing the same operation by using the same reagent as above on a solution containing previously known concentrations of CPA1, PCPA1, CPA2 and PCPA2 (total t-CPA solution), to obtain a total amount of CPA1, PCPA1, CPA2 and PCPA2 (total t-CPA amount) (concentration) in the sample. A total amount of CPA1, PCPA1, CPA2 and PCPA2 (total t-CPA amount) can also be obtained as relative value based on a proper standard (e.g. pooled serum of healthy subjects) as an amount of 100 AU (arbitrary units).

In the case that a labeled antibody (the secondary antibody) is a biotin labeled antibody, a solid phase, which is an insoluble carrier immobilized with the primary antibody, is reacted with a sample, and a biotin labeled antibody is reacted therewith to generate a biotin labeled antigen-antibody complex consisting of a biotin labeled antibody and an antigen, subsequently it is reacted with enzyme labeled streptavidin, which is labeled with an enzyme such as peroxidase, and an enzyme labeled streptavidin-a biotin labeled antigen-antibody complex is generated on the solid phase. Then a substrate for the labeled enzyme is reacted therewith and an amount of the labeled enzyme in the enzyme labeled streptavidin—the biotin labeled an antigen-antibody complex on the solid phase is assayed. Subsequently, a total amount of CPA1, PCPA1, CPA2 and PCPA2 (total t-CPA amount) in the sample can be determined from a calibration curve by the same method as described above.

A specific example of immunoassay of a total amount of CPA1, PCPA1, CPA2 and PCPA2 (total t-CPA amount) of the present invention is outlined below by exemplifying a method for assaying a total amount of CPA1, PCPA1, CPA2 and PCPA2 (total t-CPA amount) in a sample by using the peroxidase (POD) labeled secondary antibody as a labeled antibody in a coloring method.

A sample is contacted with a solid phase, which is an insoluble carrier immobilized with a t-CPA1 specific antibody and a t-CPA2 specific antibody (the primary antibody) of the present invention, to generate an antigen-antibody complex (a complex-i: four types of complexes including an immobilized t-CPA1 specific antibody/CPA1 complex, an immobilized t-CPA1 specific antibody/PCPA1 complex, an immobilized t-CPA2 specific antibody/CPA2 complex and an immobilized t-CPA2 specific antibody/PCPA2 complex) on the insoluble carrier. Subsequently, the complex is reacted with a POD labeled t-CPA1 specific antibody and a POD labeled t-CPA2 specific antibody (the secondary antibody) to generate a POD labeled antigen-antibody complex (a complex-ii: four types of complexes including an immobilized t-CPA1 specific antibody/CPA1/a POD labeled t-CPA1 specific antibody complex, an immobilized t-CPA1 specific antibody/PCPA1/a POD labeled t-CPA1 specific antibody complex, an immobilized t-CPA2 specific antibody/CPA2/a POD labeled t-CPA2 specific antibody complex and an immobilized t-CPA2 specific antibody/PCPA2/a POD labeled t-CPA2 specific antibody complex) on the insoluble carrier. Subsequently, an ODP (o-phenylenediamine dihydrochloride) solution (a coloring substance) and a H₂O₂ solution are added. After definite time from reaction start, 2N sulfuric acid is added to terminate the reaction, and then an amount of the labeling substance in the labeled antigen-antibody complex (complex-ii) on the insoluble carrier is assayed as coloring intensity (absorbancy). Thus obtained coloring intensity (absorbancy) is applied to a calibration curve showing relationship between coloring intensity (absorbancy) and a total amount of CPA1, PCPA1, CPA2 and PCPA2 (total t-CPA amount), which is obtained by performing the same operation by using the same reagent as above on a solution containing previously known concentrations of CPA1, PCPA1, CPA2 and PCPA2 (total t-CPA solution), to obtain a total amount of CPA1, PCPA1, CPA2 and PCPA2 (total t-CPA amount) in the sample.

As described above, in the above method, although assay can be performed in the same manner by using a t-CPA common antibody in place of a t-CPA1 specific antibody and a t-CPA2 specific antibody used as the primary antibody (refer to Table 2), a t-CPA1 specific antibody and a t-CPA2 specific antibody which have different epitope from the primary antibody are preferable.

Further, the same assay can be performed by exchanging the primary antibody and the secondary antibody, however, use of t-CPA common antibody as the primary antibody and a t-CPA1 specific antibody and a t-CPA2 specific antibody as the secondary antibody is preferable due to forming order of an antigen-antibody complex.

(6) Assay of a Total Amount of PCPA1 and PCPA2 (Total PCPA Amount)

Although a total PCPA amount can be assayed (calculated) by summing up an amount of PCPA1 obtained in the above (3) and an amount of PCPA2 obtained in the above (4), a total PCPA amount can be obtained by one time assay according to the following method.

A sample is contacted with a solid phase, which is an insoluble carrier immobilized with a t-CPA1 specific antibody and a t-CPA2 specific antibody (the primary antibody) of the present invention, and reacted at 4-40° C. for 3 minutes-16 hours to generate an antigen-antibody complex (a complex-i: two types of complexes consisting of an immobilized t-CPA1 specific antibody/PCPA1 complex and an immobilized t-CPA2 specific antibody/PCPA2 complex) on the insoluble carrier. Subsequently, the complex is reacted with a labeled PCPA1 specific antibody and the labeled PCPA2 specific antibody bonded with a labeling substance at 4-40° C. for 3 minutes-16 hours to generate a labeled antigen-antibody complex (a complex-ii: two types of complexes consisting of an immobilized t-CPA1 specific antibody/PCPA1/a labeled PCPA1 specific antibody complex and an immobilized t-CPA2 specific antibody/PCPA2/a labeled PCPA2 specific antibody complex) on the insoluble carrier, and then an amount of the labeling substance in the complex-ii is assayed. Thus obtained amount of labeling substance is applied to a calibration curve showing relationship between an amount of labeling substance and concentrations of PCPA1 and PCPA2 (total PCPA amount), which is obtained by performing the same operation by using the same reagent as above on a solution containing previously known concentrations of PCPA1 and PCPA2 (total PCPA solution), to obtain a total amount of PCPA1 and PCPA2 (total PCPA amount) (concentration) in the sample. The total amount of PCPA1 and PCPA2 (total PCPA amount) (concentration) may also be obtained as relative value based on a proper standard (e.g. pooled serum of healthy subjects) as an amount of 100 AU (arbitrary units).

In the case that a labeled antibody (the secondary antibody) is a biotin labeled antibody, a solid phase, which is an insoluble carrier immobilized with the primary antibody is reacted with a sample, and a biotin labeled antibody is reacted therewith to generate a biotin labeled antigen-antibody complex consisting of a biotin labeled antibody and an antigen, subsequently it is reacted with enzyme labeled streptavidin, which is labeled with an enzyme such as peroxidase, and an enzyme labeled streptavidin-a biotin labeled antigen-antibody complex is generated on the solid phase. Then a substrate for the labeled enzyme is reacted therewith and an amount of the labeled enzyme in the enzyme labeled streptavidin-a biotin labeled antigen-antibody complex on the solid phase is assayed. Subsequently, a total amount of PCPA1 and PCPA2 (total PCPA amount) in the sample can be determined from a calibration curve by the same method as described above.

A specific example of immunoassay of a total amount of PCPA1 and PCPA2 (total PCPA amount) of the present invention is outlined below by exemplifying method for assaying a total amount of PCPA1 and PCPA2 (total PCPA amount) in a sample by using the peroxidase (POD) labeled secondary antibody as a labeled an antibody in a coloring method.

A sample is contacted with a solid phase, which is an insoluble carrier immobilized with a t-CPA1 specific antibody and a t-CPA2 specific antibody (the primary antibody) of the present invention, to generate an antigen-antibody complex (a complex-i: two types of complexes consisting of an immobilized t-CPA1 specific antibody/PCPA1 complex and an immobilized t-CPA2 specific antibody/PCPA2 complex) on the insoluble carrier. Subsequently, the complex is reacted with a POD labeled PCPA1 specific antibody and a POD labeled PCPA2 specific antibody (the secondary antibody) to generate a POD labeled antigen-antibody complex (a complex-ii: two types of complexes consisting of an immobilized t-CPA1 specific antibody/PCPA1/a POD labeled PCPA1 specific antibody complex and an immobilized t-CPA2 specific antibody/PCPA2/a POD labeled PCPA2 specific antibody complex) on the insoluble carrier. An ODP (o-phenylenediamine dihydrochloride) solution (a coloring substance) and a H₂O₂ solution are added. After definite time from reaction start, 2N sulfuric acid is added to terminate the reaction, and then an amount of the labeling substance in the labeled antigen-antibody complex (complex-ii) on the insoluble carrier is assayed as coloring intensity (absorbancy). Thus obtained coloring intensity (absorbancy) is applied to a calibration curve showing relationship between coloring intensity (absorbancy) and a total amount of PCPA1 and PCPA2 (total PCPA amount), which is obtained by performing the same operation by using the same reagent as above on a solution containing previously known concentrations of PCPA1 and PCPA2, to obtain a total amount of PCPA1 and PCPA2 (total PCPA amount) in the sample.

As described above, in the above method, although assay can be performed in the same manner by using a combination of a PCPA1 specific antibody and a PCPA2 specific antibody (proviso that epitope is different from the secondary antibody), and a PCPA common antibody or a t-CPA common antibody in place of a t-CPA1 specific antibody and a t-PCA2 specific antibody used as the primary antibody (refer to Table 2), a combination of a t-CPA1 specific antibody and a t-CPA2 specific antibody is preferable.

Further, the same assay can be performed by exchanging the primary antibody and the secondary antibody, however, use of a combination of a PCPA1 specific antibody and a PCPA2 specific antibody as the secondary antibody is preferable, especially a combination of a t-CPA1 specific antibody and a t-CPA2 specific antibody as the primary antibody and a combination of a PCPA1 specific antibody and a PCPA2 specific antibody as the secondary antibody are preferable due to forming order of an antigen-antibody complex.

(7) Assay of an Amount of CPA1

An amount of CPA1 can be obtained (calculated) by subtracting an amount of PCPA1 obtained in the above (3) from a total amount of CPA1 and PCPA1 (t-CPA1 amount) obtained in the above (1).

(8) Assay of an Amount of CPA2

An amount of CPA2 can be obtained (calculated) by subtracting an amount of PCPA2 obtained in the above (4) from a total amount of CPA1 and PCPA2 (t-CPA2 amount) obtained in the above (2).

(9) Assay of a Total Amount of CPA1 and CPA2 (Total CPA Amount)

A total CPA amount can be obtained (calculated) by summing up an amount of CPA1 assayed (calculated) by subtracting an amount of PCPA1 obtained in the above (3) from a total amount of CPA1 and PCPA1 (total t-CPA1 amount) obtained in the above (1) (i.e. value obtained in the above (7)) and an amount of CPA2 assayed (calculated) by subtracting an amount of PCPA2 obtained in the above (4) from a total amount of CPA2 and PCPA2 (total t-CPA2 amount) obtained in the above (2) (i.e. value obtained in the above (8)).

As for the other immunoassay method of the present invention, latex agglutination inhibition assay (TIA) using latex particles is explained by exemplifying an assay method for various objects to be assayed in a sample.

(1) Assay of a Total Amount of CPA1 and PCPA1 (t-CPA1 Amount)

A sample and a solid phase, which is a latex particle immobilized with a t-CPA1 specific antibody (a polyclonal antibody) of the present invention, are reacted at 4-40° C. for 1 minute-24 hours, and thus generated agglutination grade (level) is measured based on changes in scattered light and transmitted light, and the result is applied to a calibration curve showing relationship between agglutination grade and concentrations of CPA1 and PCPA1 (concentration of t-CPA1), which is obtained by performing the same operation by using the same reagent as above on a solution containing previously known concentration of CPA1 and PCPA1 (t-CPA1 solution), and a total amount of CPA1 and PCPA1 (t-CPA1 amount) in the sample can be obtained. Wavelength of absorbancy measurement is generally 340-1000 nm, preferably 500-900 nm. Agglutination grade is not limited to the absorbancy value, and can be any value obtained by a known method such as values measured by using nephelometry, counting immunoassay, etc.

(2) Assay of a Total Amount of CPA2 and PCPA2 (t-CPA2 Amount)

A sample and a solid phase, which is a latex particle immobilized with a t-CPA2 specific antibody (a polyclonal antibody) of the present invention, are reacted at 4-40° C. for 1 minute-24 hours, and thus generated agglutination grade (level) is measured based on changes in scattered light and transmitted light, and the result is applied to a calibration curve showing relationship between agglutination grade and concentrations of CPA2 and PCPA2 (concentration of t-CPA2), which is obtained by performing the same operation by using the same reagent as above on a solution containing previously known concentrations of CPA2 and PCPA2 (t-CPA2 solution), and a total amount of CPA2 and PCPA2 (t-CPA2 amount) in the sample can be obtained. Wavelength of absorbancy measurement is generally 340-1000 nm, preferably 500-900 nm. Agglutination grade is not limited to the absorbancy value, and can be any value obtained by a known method such as values measured by using nephelometry, counting immunoassay, etc.

(3) Assay of an Amount of PCPA1

A sample and a solid phase, which is a latex particle immobilized with a PCPA1 specific antibody (a polyclonal antibody) of the present invention, are reacted at 4-40° C. for 1 minute-24 hours, and thus generated agglutination grade (level) is measured based on changes in scattered light and transmitted light, and the result is applied to a calibration curve showing relationship between agglutination grade and concentration of PCPA1, which is obtained by performing the same operation by using the same reagent as above on a solution containing previously known concentration of PCPA1, and an amount of PCPA1 in a sample can be obtained. Wavelength of absorbancy measurement is generally 340-1000 nm, preferably 500-900 nm. Agglutination grade is not limited to absorbancy value, and can be any value obtained by a known method such as values measured by using nephelometry, counting immunoassay, etc.

(4) Assay of an Amount of PCPA2

A sample and a solid phase, which is a latex particle immobilized with the PCPA2 specific antibody (a polyclonal antibody) of the present invention, are reacted at 4-40° C. for 1 minute-24 hours, and thus generated agglutination grade (level) is measured based on changes in scattered light and transmitted light, and the result is applied to a calibration curve showing relationship between agglutination grade and concentration of PCPA2, which is obtained by performing the same operation by using the same reagent as above on a solution containing previously known concentration of PCPA2, and an amount of PCPA2 in a sample can be obtained. Wavelength of absorbancy measurement is generally 340-1000 nm, preferably 500-900 nm. Agglutination grade is not limited to absorbancy value, and can be any value obtained by a known method such as values measured by using nephelometry, counting immunoassay, etc.

(5) Assay of a Total Amount of CPA1, CPA2, PCPA1 and PCPA2 (Total t-CPA Amount)

A total t-CPA amount can be obtained (calculated) by summing up a total amount of CPA1 and PCPA1 (t-CPA1 amount) obtained in the above (1) and a total amount of CPA2 and PCPA2 (t-CPA2 amount) obtained in the above (2). In addition, using a combination of the same antibody (the primary antibody and the secondary antibody) as described in an assay method (5) for various objects to be assayed in samples by the above-described coloring method, a total t-CPA amount can be assayed by one time assay.

(6) Assay of a Total Amount of PCPA1 and PCPA2 (Total PCPA Amount)

A total PCPA amount can be obtained (calculated) by summing up an amount of PCPA1 obtained in the above (3) and an amount of PCPA2 obtained in the above (4). In addition, using a combination of the same antibody (the primary antibody and the secondary antibody) as described in an assay method (6) for various objects to be assayed in samples by the above-described coloring method, a total t-CPA amount can be assayed by one time assay.

(7) Assay of an Amount of CPA1

An amount of CPA1 can be obtained (calculated) by subtracting an amount of PCPA1 obtained in the above (3) from a total amount of CPA1 and PCPA1 (t-CPA1 amount) obtained in the above (1).

(8) Assay of an Amount of CPA2

An amount of CPA2 can be obtained (calculated) by subtracting an amount of PCPA2 obtained in the above (4) from a total amount of CPA2 and PCPA2 (t-CPA2 amount) obtained in the above (2).

(9) Assay of a Total Amount of CPA1 and CPA2 (Total CPA Amount)

A total CPA amount can be obtained (calculated) by summing up a amount of CPA1 assayed (calculated) by subtracting an amount of PCPA1 obtained in the above (3) from a total amount of CPA1 and PCPA1 (t-CPA1 amount) obtained in the above (1) (i.e. value obtained in the above (7)),and an amount of CPA2 assayed (calculated) by subtracting an amount of PCPA2 obtained in the above (4) from a total amount of CPA2 and PCPA2 (t-CPA2 amount) obtained in the above (2) (i.e. value obtained in the above (8)).

As for the other immunoassay method of the present invention, among immunoassay methods by means of electrophoresis utilizing an antigen-antibody reaction, assay methods for various objects to be assayed in a sample using an antibody and a conjugate (a complex with an antibody and a separation improving substance) and further using micro fluidic device is explained as follows.

(1) Assay of a Total Amount of CPA1 and PCPA1 (t-CPA1 Amount)

A sample is contacted with (i) a t-CPA1 specific antibody of the present invention (the primary antibody) and (ii) a conjugate of a separation improving substance (e.g. nucleic acid chain of 1 pb -1000 kbp) and a t-CPA1 specific antibody (the secondary antibody: proviso that epitope is different from the primary antibody) (a separation improving substance bound t-CPA1 specific antibody), in which at least either one of the primary antibody or the conjugate is labeled by a labeling substance, and reacted at 0-90° C. for 1 sec.-24 hours to form an antigen-antibody complex of an object to be assayed, an antibody and the conjugate (two types of labeled complexes of a labeled t-CPA1 specific antibody/CPA1 complex/a conjugate (a separation improving substance bonded t-CPA1 specific antibody) complex and a labeled t-CPA1 specific antibody/PCPA1/a conjugate (a separation improving substance bound t-CPA1 specific antibody) complex, or two types of labeled complexes of a t-CPA1 specific antibody/CPA1 complex/a labeled conjugate (a labeling separation improving substance bound t-CPA1 specific antibody) complex, and a t-CPA1 specific antibody/PCPA1/a labeled conjugate (a labeled separation improving substance bound t-CPA1 specific antibody) complex). Subsequently, a labeled complex is separated from a free labeled t-CPA1 specific antibody or a labeled conjugate (a labeling separation improving substance bound t-CPA1 specific antibody), which are not involved in complex formation, if necessary, in the presence of a charged polymer (e.g. a polyanion), in a separation channel of micro fluidic device to assay an amount of the labeling substance in the complex. Thus obtained amount of the labeling substance is applied to a calibration curve showing relationship between an amount of the labeling substance and concentrations of CPA1 and PCPA1 (concentration of t-CPA1), which is obtained by performing the same operation by using the same reagent as above on a solution containing previously known concentrations of CPA1 and PCPA1 (t-CPA1 solution), and a total amount of CPA1 and PCPA1 in a sample (t-CPA1 amount) (concentration) can be obtained. Further, a detectable substance (e.g. nucleic acid labeled with a labeling substance, etc.) of known concentration is added as an internal standard in a sample, and a relative total amount of CPA1 and PCPA1 in a sample (t-CPA1 amount) (concentration) is calculated by comparing with an amount of the detectable substance added as the internal standard and an amount of the labeling substance in the complex. A total amount of CPA1 and PCPA1 (t-CPA1 amount) (concentration) can also be obtained as relative value based on a proper standard (e.g. pooled serum of healthy subjects) as an amount of 100 AU (arbitrary units).

In the above method, use of separation device, electric power supply for electrophoresis, reagents such as a buffer solution, fillers, solutions for various treatments, concentration thereof to be used, capillary material, various separation conditions (e.g. pH, temperature, applied voltage, time, etc.) can be determined by known methods.

As described above, in the above method, although assay can be performed in the same manner by using a t-CPA common antibody in place of a t-CPA1 specific antibody used as the primary antibody (refer to Table 2), a t-CPA1 specific antibody which has different epitope from the primary antibody is preferable.

Further, the same assay can be performed by exchanging the primary antibody and the secondary antibody, however, use of a t-CPA1 specific antibody as the primary antibody and a t-CPA1 specific antibody as the secondary antibody is preferable due to forming order of an antigen-antibody complex.

(2) Assay of a Total Amount of CPA2 and PCPA2 (t-CPA2 Amount)

A sample is contacted with (i) a t-CPA2 specific antibody of the present invention (the primary antibody) and (ii) a conjugate of a separation improving substance (e.g. nucleic acid chain of 1 pb -1000 kbp) and a t-CPA2 specific antibody (the secondary antibody: proviso that epitope is different from the primary antibody) (a separation improving substance bound t-CPA2 specific antibody), in which at least one of the primary antibody or a conjugate is labeled by a labeling substance, and reacted at 0-90° C. for 1 sec.-24 hours to form an antigen-antibody complex of an object to be assayed, an antibody and a conjugate (two types of labeled complexes of a labeled t-CPA2 specific antibody/CPA2 complex/a conjugate (a separation improving substance bound t-CPA2 specific antibody) complex and a labeled t-CPA2 specific antibody/PCPA2/a conjugate (a separation improving substance bound t-CPA2 specific antibody complex, or two types of labeled complexes of a t-CPA2 specific antibody/CPA2 complex/a labeled conjugate (a labeled separation improving substance bound t-CPA2 specific antibody) complex, and a t-CPA2 specific antibody/PCPA2/a labeled conjugate (a labeled separation improving substance bound t-CPA2 specific antibody) complex). Subsequently, the labeled complex is separated from a free labeled t-CPA2 specific antibody or a labeled conjugate (a labeling separation improving substance bonded t-CPA2 specific antibody), which are not involved in complex formation, if necessary, in the presence of a charged polymer (e.g. a polyanion), in a separation channel of micro fluidic device to assay an amount of the labeling substance in the complex. Thus obtained amount of the labeling substance is applied to a calibration curve showing relationship between an amount of the labeling substance and concentrations of CPA2 and PCPA2 (concentration of t-CPA2), which is obtained by performing the same operation by using the same reagent as above on a solution containing previously known concentrations of CPA2 and PCPA2 (t-CPA2 solution), and a total amount of CPA2 and PCPA2 in a sample (t-CPA2 amount) (concentration) can be obtained. Further, a detectable substance (e.g. nucleic acid labeled with labeling substance, etc.) of known concentration is added as an internal standard in a sample, and a relative total amount of CPA2 and PCPA2 in a sample (t-CPA2 amount) (concentration) is calculated by comparing with an amount of the detectable substance added as the internal standard and an amount of the labeling substance in the complex. A total amount of CPA2 and PCPA2 (t-CPA2 amount) (concentration) can also be obtained as relative value based on a proper standard (e.g. pooled serum of healthy subjects) as an amount of 100 AU (arbitrary units)

In the above method, separation device used, electric power supply for electrophoresis, reagents such as a buffer solution, fillers, solutions for various treatments, concentration thereof to be used, capillary material, various separation conditions (e.g. pH, temperature, applied voltage, time, etc., can be determined by known methods.

As described above, in the above method, although assay can be performed in the same manner by using a t-CPA common antibody in place of a t-CPA2 specific antibody used as the primary antibody (refer to Table 2), a t-CPA2 specific antibody which has different epitope from the primary antibody is preferable.

Further, the same assay can be performed by exchanging the primary antibody and the secondary antibody, however, use of a t-CPA2 specific antibody as the primary antibody and a t-CPA2 specific antibody as the secondary antibody is preferable due to forming order of an antigen-antibody complex.

(3) Assay of an Amount of PCPA1

A sample is contacted with (i) a t-CPA1 specific antibody of the present invention (the primary antibody) and (ii) a conjugate of a separation improving substance (e.g. nucleic acid chain of 1 pb -1000 kbp) and a PCPA1 specific antibody (a separation improving substance bound PCPA1 specific antibody), in which at least either of the primary antibody or a conjugate is labeled by a labeling substance, and reacted at 0-90° C. for 1 sec.-24 hours to form an antigen-antibody complex of an object to be assayed, an antibody and a conjugate (a complex of a labeled t-CPA1 specific antibody/PCPA1/a conjugate (a separation improving substance bound PCPA1 specific antibody) complex or a t-CPA1 specific antibody/PCPA1/a labeled conjugate (a labeled separation improving substance bound PCPA1 specific antibody) complex) . Subsequently, the labeled complex is separated from a free labeled t-CPA1 specific antibody or a labeled conjugate (a labeled separation improving substance bound PCPA1 specific antibody), which are not involved in complex formation, if necessary, in the presence of a charged polymer (e.g. a polyanion), in a separation channel of micro fluidic device to assay an amount of the labeling substance in the complex. Thus obtained amount of the labeling substance is applied to a calibration curve showing relationship between an amount of the labeling substance and concentration of PCPA1, which is obtained by performing the same operation by using the same reagent as above on a solution containing previously known concentration of PCPA1, and an amount of PCPA1 in the sample (concentration) can be obtained. Further, a detectable substance (e.g. nucleic acid labeled with a labeling substance, etc.) of known concentration is added as an internal standard in the sample, and a relative amount of PCPA1 in the sample (concentration) is calculated by comparing with an amount of the detectable substance added as the internal standard and an amount of the labeling substance in the complex. An amount of PCPA1 (concentration) can also be obtained as relative value based on a proper standard (e.g. pooled serum of healthy subjects) as an amount of 100 AU (arbitrary units).

In the above method, separation device used, electric power supply for electrophoresis, reagents such as a buffer solution, fillers, solutions for various treatments, concentration thereof to be used, capillary material, various separation conditions (e.g. pH, temperature, applied voltage, time, etc., can be determined by known methods.

As described above, in the above method, although assay can be performed in the same manner by using a PCPA1 specific antibody (proviso that epitope is different from the secondary antibody), a PCPA common antibody or a t-CPA common antibody in place of a t-CPA1 specific antibody used as the primary antibody (refer to Table 2), a t-CPA1 specific antibody is preferable.

Further, the same assay can be performed by exchanging the primary antibody and the secondary antibody, however, use of PCPA1 specific antibody as the secondary antibody is preferable, and especially use of a t-CPA1 specific antibody as the primary antibody and a PCPA1 specific antibody as the secondary antibody is preferable due to forming order of an antigen-antibody complex.

(4) Assay of an Amount of PCPA2

A sample is contacted with (i) a t-CPA2 specific antibody of the present invention (the primary antibody) and (ii) a conjugate of a separation improving substance (e.g. nucleic acid chain of 1 pb -1000 kbp) and a PCPA2 specific antibody (a separation improving substance bound PCPA2 specific antibody), in which at least either one of the primary antibody or a conjugate is labeled by a labeling substance, and reacted at 0-90° C. for 1 sec.-24 hours to form an antigen-antibody complex of an object to be assayed, an antibody and a conjugate (a complex of a labeled t-CPA2 specific antibody/PCPA2/a conjugate (a separation improving substance bound PCPA2 specific antibody) complex or a t-CPA2 specific antibody/PCPA2/a labeled conjugate (a labeled separation improving substance bound PCPA2 specific antibody) complex) . Subsequently, the labeled complex is separated from a free labeled t-CPA2 specific antibody or a labeled conjugate (a labeled separation improving substance bound PCPA2 specific antibody), which are not involved in complex formation, if necessary, in the presence of a charged polymer (e.g. a polyanion), in a separation channel of micro fluidic device to assay an amount of the labeling substance in the complex. Thus obtained amount of the labeling substance is applied to a calibration curve showing relationship between an amount of the labeling substance and concentration of PCPA2, which is obtained by performing the same operation by using the same reagent as above on a solution containing previously known concentration of PCPA2, and an amount of PCPA2 in the sample (concentration) can be obtained. Further, a detectable substance (e.g. nucleic acid labeled with a labeling substance, etc.) of known concentration is added as an internal standard in the sample, and a relative amount of PCPA2 in the sample (concentration) is calculated by comparing with an amount of the detectable substance added as the internal standard and an amount of the labeling substance in the complex. A amount of PCPA2 (concentration) can also be obtained as relative value based on a proper standard (e.g. pooled serum of healthy subjects) as an amount of 100 AU (arbitrary units).

In the above method, used separation device, electric power supply foe electrophoresis, reagents such as a buffer solution, fillers, solutions for various treatments, concentration to be used, capillary material, various separation conditions (e.g. pH, temperature, applied voltage, time, etc., can be determined by known methods.

As described above, in the above method, although assay can be performed in the same manner by using a PCPA2 specific antibody (proviso that epitope is different from the secondary antibody), a PCPA common antibody or a t-CPA common antibody in place of a t-CPA2 specific antibody used as the primary antibody (refer to Table 2), a t-CPA2 specific antibody is preferable.

Further, the same assay can be performed by exchanging the primary antibody and the secondary antibody, however, use of PCPA2 specific antibody as the secondary antibody is preferable, and especially use of a t-CPA2 specific antibody as the primary antibody and a PCPA2 specific antibody as the secondary antibody is preferable due to forming order of an antigen-antibody complex.

(5) Assay of a Total Amount of CPA1, CPA2, PCPA1 and PCPA2 (Total t-CPA Amount)

A total t-CPA amount can be obtained (calculated) by summing up a total amount of CPA1 and PCPA1 (t-CPA1 amount) obtained in the above (1) and a total amount of CPA2 and PCPA2 (t-CPA2 amount) obtained in the above (2). In addition, using a combination of the same antibody (the primary antibody and the secondary antibody) as described in an assay method (5) for various objects to be assayed in samples by the above-described coloring method, a total t-CPA amount can be assayed by one time assay.

(6) Assay of a Total Amount of PCPA1 and PCPA2 (Total PCPA Amount)

A total PCPA amount can be obtained (calculated) by summing up an amount of PCPA1 obtained in the above (3) and an amount of PCPA2 obtained in the above (4). In addition, using a combination of the same antibody (the primary antibody and the secondary antibody) as described in an assay method (6) for various objects to be assayed in samples by the above-described coloring method, a cumulative amount of PCPA can be assayed by one time assay.

(7) Assay of an Amount of CPA1

An amount of CPA1 can be obtained (calculated) by subtracting an amount of PCPA1 obtained in the above (3) from a total amount of CPA1 and PCPA1 (t-CPA1 amount) obtained in the above (1).

(8) Assay of an Amount of CPA2

An amount of CPA2 can be obtained (calculated) by subtracting an amount of PCPA2 obtained in the above (4) from a total amount of CPA2 and PCPA2 (t-CPA2 amount) obtained in the above (2).

(9) Assay of a Total Amount of CPA1 and CPA2 (Total CPA Amount)

A total CPA amount can be obtained (calculated) by summing up an amount of CPA1 assayed (calculated) by subtracting an amount of PCPA1 obtained in the above (3) from a total amount of CPA1 and PCPA1 (t-CPA1 amount) obtained in the above (1) (i.e. value obtained in the above (7)), and an amount of CPA2 assayed (calculated) by subtracting an amount of PCPA2 obtained in the above (4) from a total amount of CPA2 and PCPA2 (t-CPA2 amount) obtained in the above (2) (i.e. value obtained in the above (8)).

2-11. A Sample

Examples of a sample used in the present invention are derived from organisms such as various body fluids (e.g. blood, serum, plasma, spinal fluid, pancreatic juice, synovial fluid, lymph, etc.), excretions (e.g. urine, saliva, etc.), lymphocytes, blood cells, various cells, tissue extracts and treated materials prepared by reconstruction thereof dissolved or suspended in water or buffer, commonly used in this field (e.g. Tris buffer, phosphate buffer, veronal buffer, borate buffer, Good's buffer, etc.).

Examples of buffer used in an assay method of the present invention are those generally used for assay methods applying an antigen-antibody reaction, for example Tris buffer, phosphate buffer, veronal buffer, borate buffer, Good's buffer, etc. pH is not limited, if it is within the range for not to inhibit an antigen-antibody reaction, and is generally preferably within pH range of 5-9.

The present invention can be applied sufficiently not only to manual means but also for an assay system using an automatic analyzer, and assay can be made easily and rapidly. A combination of reagents, etc., for use in assay using an automatic analyzer is not especially limited, and most preferable combination of reagents, and the like can be selected properly by considering equipment and other factors.

2-12. Effect of an Assay Method of the Present Invention

An assay method of the present invention is based on immunoassay using a specific antibody of the present invention as described above, and is not based on an assay method of enzyme activity assaying enzymatic activity of CPA using substrates of CPA (i.e. in a method of the present invention, substrates for CPA may not be used). Consequently, an assay method of the present invention has the following effects:
(1) An object to be assayed and a subject other than the object can be discriminated, and the object to be assayed can be specifically and simply assayed. (refer to the above 1-4.)
(2) Comparing with an assay method of enzyme activity, which is easily affected by measurement conditions (e.g. temperature, pH, etc.), a method of the present invention is little affected by such conditions.
(3) Since substrates for CPA are not used, there are no problems on variation in assay values caused by substrate types, and objects to be assayed can be measured easily and highly precisely.
(4) Since assay is performed not for activity of objects to be assayed but for directly assaying an amount thereof, objects to be assayed can be measured in high precision.
(5) CPA, which is denatured in its enzyme activity (e.g. CPA (CPA1 and CPA2) in samples derived from patients after treatment by such as an enzyme inhibitor, etc.), or mutated CPA (mutated CPA1 and mutated CPA1) can also be assayed in high precision.

Conventionally known methods for obtaining PCPA activity (Peterson et al. Biochemistry, 22: 3077-3082, 1983; Published Japanese translation of PCT international publication for patent application No. JP-A-2001-518791; Clinica Chimica Acta 2000, 292: 107-115) is not only impossible to differentiate PCPA1 and PCPA2 but also a complex and complicated method for assaying PCPA (PCPA1 and PCPA2) due to presence of the following three steps.

(1) Pro-region of PCPA (total PCPA: PCPA1 and PCPA2) is removed by using proteinase to convert activated CPA, and total (total t-CPA) activity of CPA converted from PCPA and originally existing CPA (total CPA: CPA1 and CPA2) is assayed.

(2) Assay is performed without using proteinase in the same way as in (1) to assay only originally existing CPA (total CPA: CPA1 and CPA2).

(3) Total CPA (CPA1 and CPA2) activity obtained in the above (2) is subtracted from total t-CPA (PCPA1, PCPA2, CPA1 and CPA2) activity obtained in the above (1) to obtain total PCPA (PCPA1 and PCPA2) activity.

Contrary to that, a method of the present invention is not required such complex and complicated procedures, and generally two steps or less, preferably one step operation is able to provide measurement of objects to be assayed (e.g. PCPA1, PCPA2, t-CPA1, t-CPA2, etc.) simply and easily.

Further, since such a method of the present invention is simple and easy in measurement operation (steps) compared with conventional assay methods, required time from assay initiation to termination thereof is shorter than that in a conventional method, and in addition, sample volume and reagent volume for use are quite smaller as compared with a conventional method, consequently a method of the present invention has economically advantage.

3. A Judgment Method for Pancreas Cancer

3-1. A Judgment Method for Pancreas Cancer of the Present Invention

A judgment method for pancreas cancer of the present invention comprises a method wherein one or more amount selected from an amount of CPA1 in a sample, an amount of CPA2 in a sample, an amount of PCPA1 in a sample or an amount of PCPA2 in a sample is assayed and “whether it is pancreas cancer or not” is judged based on thus obtained value.

Specifically, a method includes the following: (A) a judgment method for pancreas cancer based on difference obtained by comparing (1) one or more amount selected from an amount of CPA1 in a sample, an amount of CPA2 in a sample, an amount of PCPA1 in a sample and an amount of PCPA2 in a sample, and (2) an amount of the same in control; (B) a judgment method for pancreas cancer based on amount ratio of (1) below and a cumulative amount of (2) below obtained by assaying (1) one or more amounts of enzymes selected from an amount of CPA1 in a sample, an amount of CPA2 in a sample, an amount of PCPA1 in a sample and an amount of PCPA2 in a sample, and (2) one or more total amounts of enzymes selected from (a) a total amount of CPA1 and PCPA1 (t-CPA1), (b) a total amount of CPA2 and PCPA2 (t-CPA2), (c) a total amount of CPA1 and CPA2 (cumulative CPA), (d) a total amount of PCPA1 and PCPA2 (cumulative PCPA) and (e) a total amount of CPA1, CPA2, PCPA1 and PCPA2 (cumulative t-CPA); and (C) a judgment method for pancreas cancer based on amount ratio of (1) below and an amount of (2) below, obtained by assaying (1) one type of an amount of an enzyme selected from an amount of CPA1 in a sample, an amount of CPA2 in a sample, an amount of PCPA1 in a sample or an amount of PCPA2 in a sample, and (2) one type of an amount of enzyme selected from residual amounts of enzymes of (1).

3-2. Objects to be Assayed in a Judgment Method of the Present Invention (An Assay Method)

“One kind of an amount selected from an amount of CPA1 in a sample, an amount of CPA2 in a sample, an amount of PCPA1 in a sample or an amount of PCPA2 in a sample” ((1) in (A) to (C) above) assayed in a judgment method of the present invention (an object to be assayed) means an amount of CPA1 in a sample, an amount of CPA2 in a sample, an amount of PCPA1 in a sample or an amount of PCPA2 in a sample, a total amount of CPA1 and PCPA1 (t-CPA1), a total amount of CPA2 and PCPA2 (t-CPA2), a total amount of CPA1 and CPA2 (total CPA), a total amount of PCPA1 and PCPA2 (total pro CPA) and a total amount of CPA1, CPA2, PCPA1 and PCPA2 (total t-CPA). Among them, an amount of CPA1 in a sample, an amount of CPA2 in a sample, an amount of PCPA1 in a sample or an amount of PCPA2 in a sample, a total amount of CPA1 and PCPA1 (t-CPA1) or a total amount of CPA2 and PCPA2 (t-CPA2) are especially preferable.

Objects to be assayed in a judgment method of the present invention as described above can be assayed (calculated) as follows.

(1) A Total Amount of CPA1 and PCPA1 (t-CPA1 Amount)

Assay can be made by immunoassay of the present invention as described above.

(2) A Total Amount of CPA2 and PCPA2 (t-CPA2 Amount)

Assay can be made by immunoassay of the present invention as described above.

(3) An Amount of PCPA1

Assay can be made by immunoassay of the present invention as described above.

(4) An Amount of PCPA2

Assay can be made by immunoassay of the present invention as described above.

(5) A Total Amount of CPA1, CPA2, PCPA1 and PCPA2 (Total t-CPA Amount)

Assay can be made by immunoassay of the present invention as described above. Assay (calculation) can also be made by summing up a total amount of CPA1 and PCPA1 (t-CPA1 amount) obtained in above (1) and a total amount of CPA2 and PCPA2 (t-CPA2 amount) obtained in above (2). Further, assay can be made by immunoassay using a known t-CPA common antibody (an antibody bound to both CPA1 and CPA2).

(6) A Total Amount of PCPA1 and PCPA2 (Total PCPA Amount)

Assay can be made by immunoassay of the present invention as described above. Assay (calculation) can also be made by summing up an amount of PCPA1 obtained in above (3) and an amount of PCPA2 obtained in above (4). Further, assay can be made by immunoassay using a known PCPA common antibody (an antibody bound to both PCPA1 and PCPA2).

(7) An Amount of CPA1

Assay can be made by immunoassay of the present invention as described above.

(8) An Amount of CPA2

Assay can be made by immunoassay of the present invention as described above.

(9) A Total Amount of CPA1 and CPA2 (Total CPA Amount)

Assay can be made by immunoassay of the present invention as described above. Assay (calculation) can also be made by subtracting an amount (total PCPA amount) obtained by immunoassay using a known PCPA common antibody (an antibody bound to both PCPA1 and PCPA2) from an amount obtained by immunoassay using a known t-CPA common antibody (an antibody bound to both CPA1 and CPA2).

Objects to be assayed may be assayed (calculated) separately or simultaneously in one time assay.

3-3. A Indicator for Judgment of Pancreas Cancer

in a judgment method of the present invention, pancreas cancer or not can be judged based on an amount (value) of an object to be assayed obtained by the above method.

That is, in a judgment method of the present invention, an amount (value) of an object to be assayed itself obtained by the above method or amount ratio (proportion) of two objects to be assayed is used as a judgment indicator for pancreas cancer and pancreas cancer or not is judged.

In a judgment method of the present invention, a judgment indicator used for pancreas cancer is typically as follows, as shown in Table 3: (1) One of an object amount selected from an amount of CPA1, an amount of CPA2, an amount of PCPA1, an amount of PCPA2, a total amount of CPA1 and PCPA1 (t-CPA1), a total amount of CPA2 and PCPA2 (t-CPA2), a total amount of CPA1 and CPA2 (total CPA), a total amount of PCPA1 and PCPA2 (total pro CPA) and a total amount of CPA1, CPA2, PCPA1 and PCPA2 (total t-CPA); (2) amount ratio of the same objects among isozymes (CPA1 and CPA2, PCPA1 and PCPA2, or t-CPA1 and t-CPA2); (3) amount ratio of a matured enzyme and proenzyme; (4) amount ratio of total amount of a matured enzyme and proenzyme, and an amount of a matured enzyme; (5) amount ratio of total amount of a matured enzyme and proenzyme, and an amount of proenzyme; (6) amount ratio of total amount of an isozymes and an amount of one isozyme; (7) amount ratio of total amount of a matured isozyme and pro-isozyme (total t-CPA amount) and an amount of one isozyme; or (8) amount ratio of total amount of a matured enzyme and proenzyme and a total amount of isozymes.

TABLE 3
(1) Amount of one object is used as a indicator for judgment:

| | |
|---|---|
| 1 | (7) amount of CPA1 |
| 2 | (8) amount of CPA2 |
| 3 | (3) amount of PCPA1 |
| 4 | (4) amount of PCPA2 |
| 5 | (1) amount of t-CPA1 |
| 6 | (2) amount of t-CPA2 |
| 7 | (9) total CPA amount |
| 8 | (6) total PCPA amount |
| 9 | (5) total t-CPA amount |

(2) Ratio of amount of the same objects among isozymes is used as a
indicator for judgment:

| | |
|---|---|
| 10 | Ratio of (7) amount of CPA1 and (8) amount of CPA2 |
| 11 | Ratio of (3) amount of PCPA1 and (4) amount of PCPA2 |
| 12 | Ratio of (1) amount of t-CPA1 and (2) amount of t-CPA2 |

(3) Ratio of amount of matured enzyme and amount of proenzyme is used
as a indicator for judgment:
a) Same isozyme

| | |
|---|---|
| 13 | Ratio of (7) amount of CPA1 and (3) amount of PCPA1 |
| 14 | Ratio of (8) amount of CPA2 and (4) amount of PCPA2 |

b) Among different isozymes

| | |
|---|---|
| 15 | Ratio of (7) amount of CPA1 and (4) amount of PCPA2 |
| 16 | Ratio of (8) amount of CPA2 and (3) amount of PCPA1 |

c) Total amount of isozymes

| | |
|---|---|
| 17 | Ratio of (9) total CPA amount and (6) total PCPA amount |

(4) Ratio of amount of total amount of matured enzyme and proenzyme
and amount of matured enzyme is used as a indicator for judgment:
a) Same isozyme

| | |
|---|---|
| 18 | Ratio of (7) amount of CPA1 and (1) amount of t-CPA1 |
| 19 | Ratio of (8) amount of CPA2 and (2) amount of t-CPA2 |

b) Among different isozymes

| | |
|---|---|
| 20 | Ratio of (7) amount of CPA1 and (2) amount of t-CPA2 |
| 21 | Ratio of (8) amount of CPA2 and (1) amount of t-CPA1 |

(5) Ratio of amount of total amount of matured enzyme and proenzyme
and amount of proenzyme is used as a marker for judgment:
a) Same isozyme

| | |
|---|---|
| 22 | Ratio of (3) amount of PCPA1 and (1) amount of t-CPA1 |
| 23 | Ratio of (4) amount of PCPA2 and (2) amount of t-CPA2 |

b) Among different isozymes

| | |
|---|---|
| 24 | Ratio of (3) amount of PCPA1 and (2) amount of t-CPA2 |
| 25 | Ratio of (4) amount of PCPA2 and (1) amount of t-CPA1 |

(6) Ratio of an amount of total amount of isozymes and an amount of one
isozyme is used as a marker for judgment:
a) Matured enzyme

| | |
|---|---|
| 26 | Ratio of (7) amount of CPA1 and (9) total CPA amount |
| 27 | Ratio of (8) amount of CPA2 and (9) total CPA amount |

b) Proenzyme

| | |
|---|---|
| 28 | Ratio of (3) amount of PCPA1 and (6) total PCPA amount |
| 29 | Ratio of (4) amount of PCPA2 and (6) total PCPA amount |

c) Among different forms (matured enzyme and proenzyme)

| | |
|---|---|
| 30 | Ratio of (7) amount of CPA1 and (6) total PCPA amount |
| 31 | Ratio of (8) amount of CPA2 and (6) total PCPA amount |
| 32 | Ratio of (3) amount of PCPA1 and (9) total CPA amount |
| 33 | Ratio of (4) amount of PCPA2 and (9) total CPA amount |

(7) a ratio of amount of total amount of matured isozyme and
pro-isozyme (total t-CPA amount) and an amount of one isozyme
is used as a indicator for judgment:
a) Matured isozyme

| | |
|---|---|
| 34 | Ratio of (7) amount of CPA1 and (5) total t-CPA amount |
| 35 | Ratio of (8) amount of CPA2 and (5) total t-CPA amount |

b) Pro-isozyme

| | |
|---|---|
| 36 | Ratio of (3) amount of PCPA1 and (5) total t-CPA amount |
| 37 | Ratio of (4) amount of PCPA2 and (5) total t-CPA amount |

c) Total amount of matured enzyme and proenzyme in an isozyme

| | |
|---|---|
| 38 | Ratio of (1) amount of t-CPA1 and (5) total t-CPA amount |
| 39 | Ratio of (2) amount of t-CPA2 and (5) total t-CPA amount |

d) Total amount of isozyme in matured enzyme and proenzyme

| | |
|---|---|
| 40 | Ratio of (9) cumulative amount of CPA and (5) total t-CPA amount |
| 41 | Ratio of (6) cumulative amount of PCPA and (5) total t-CPA amount |

(8) a ratio of amount of total amount of matured enzyme and proenzyme
and a total amount of isozymes is used as a indicator for judgment:
a) Matured enzyme

| | |
|---|---|
| 42 | Ratio of (1) amount of t-CPA1 and (9) total CPA amount |
| 43 | Ratio of (2) amount of t-CPA2 and (9) total CPA amount |

b) Proenzyme

| | |
|---|---|
| 44 | Ratio of (1) amount of t-CPA1 and (6) total PCPA amount |
| 45 | Ratio of (2) amount of t-CPA2 and (6) total PCPA amount |

Among judgment indicators of the present invention, an amount of PCPA1, an amount of PCPA2, an amount of t-CPA1, an amount of t-CPA2, a total CPA amount and a total PCPA amount are preferable, and an amount of PCPA1, an amount of PCPA2, an amount of t-CPA1 and an amount of t-CPA2 are more preferable, and an amount of PCPA1 and an amount of t-CPA1 are most preferable.

3-4. Judgment of Pancreas Cancer

Judgment of pancreas cancer using the above judgment indicators (amounts (values) of objects to be assayed itself obtained by the above method, or amount ratio (proportion) of two objects to be assayed) can be made, for example, as follows: A judgment indicator obtained by assay and control (standard value) determined based on a judgment indicator in a sample derived from a healthy subject (an amount (value) of an object to be assayed itself obtained by the above method, or amount ratio (proportion) of two objects to be assayed) are compared. Then “difference between a judgment indicator obtained by assay and control (standard value)” (i.e. whether a judgment indicator obtained as a result of assay is significantly different from control (standard value)) is judged, and if significant difference is found (i.e. significantly high (or low)), a case is judged to be pancreas cancer.

(1) An amount of an object to be assayed in samples derived from healthy subjects (i.e. an amount of CPA1, an amount of CPA2, an amount of PCPA1, an amount of PCPA2, a total amount of CPA1 and PCPA1 (t-CPA1), a total amount of CPA2 and PCPA2 (t-CPA2), a total amount of CPA1 and CPA2 (total CPA), a total amount of PCPA1 and PCPA2 (total pro CPA) and a total amount of CPA1, CPA2, PCPA1 and PCPA2 (total t-CPA)) is assayed (calculated) by the above method (preferably immunoassay of the present invention), and the amount (value) itself or amount ratio (proportion) of two objects selected therefrom is used as control (standard value); (2) an amount of an object to be assayed in a sample to be judged is assayed (calculated) by the above method (preferably immunoassay of the present invention), and an amount (value) itself or amount ratio (proportion) of two objects selected therefrom is used as a judgment indicator; and (3) a judgment indicator is determined to be significantly different or not as compared with control (standard value), then a case, indicating significantly high (or low), is judged as having pancreas cancer.

A judgment method of the present invention typically includes the following:
(1) An amount of an object to be assayed in a sample of healthy subjects is assayed according to an assay method of the present invention. The amount (value) itself of the object to be assayed, or ratio (proportion) of two objects to be assayed selected therefrom is set as control (standard value) (100 arbitrary units (AU)). Separately, an amount of an object to be assayed in a sample to be judged is assayed by the same manner, and an amount of the object (value) to be assayed or amount ratio (proportion) of two objects to be assayed selected therefrom (a judgment indicator) is determined. Relative value of a judgment indicator to control (standard value) is calculated. Based on the result, “whether a significant difference between thus obtained relative value and control value (normal value) can be observed or not” is judged, and a case with significantly high (or a case showing low) difference is judged to be pancreas cancer.
(2) An amount of an object to be assayed in a sample of healthy subjects (assayed value of absorbancy, level of luminescence, etc.) is assayed according to an assay method of the present invention. The amount (value) itself of an object to be assayed, or value ratio (proportion) of two objects to be assayed selected therefrom is set as control (standard value). Separately, an amount of an object to be assayed (assayed value of absorbance, level of luminescence, etc.) in a sample to be judged is assayed by the same manner, and value of an object to be assayed or value ratio (proportion) of two objects to be assayed selected therefrom (a judgment indicator) is determined. Based on the results, “whether a significant difference between a judgment marker and control (standard value) can be observed or not” is judged, and a case with significantly high (or a case showing low) difference is judged to be pancreas cancer.
(3) An assay according to the present invention is conducted by using a sample (a standard solution) containing at least one selected from an amount of CPA1, an amount of CPA2, an amount of PCPA1 and an amount of PCPA2 with known concentration. A calibration curve (a calibration curve relating to an amount (value) itself of object to be assayed, or amount ratio (proportion) of two objects to be assayed) is prepared by using thus obtained assay results. Separately, an assay method of the present invention is performed by using samples of healthy subjects. An amount (value) of an object to be assayed or amount ratio (proportion) of two objects to be assayed obtained from a calibration curve is statistically analyzed to obtain normal range. Further, an assay is performed similarly by using a sample to be judged and an amount (value) of object to be assayed or amount ratio (proportion) of two objects to be assayed from a calibration curve is obtained. In the case that thus obtained value is significantly higher (or lower) than that of normal range, the case is judged as pancreas cancer.

Statistical analysis used for judgment of significant difference can be performed, for example, by analysis of variance (ANOVA) using Fisher's least significant difference (Fisher's PLSD) for difference between two groups, and Bartlett's test is used for statistical test of correlation.

In a judgment method of the present invention to judge pancreas cancer, especially pancreas cancer in the initial state, relative value of a judgment indicator to control (standard value) is obtained, then “whether the relative value is significantly different from control (standard value) or not” is judged. Such standard value include; a mean value of a judgment indicator obtained from a group of samples derived from healthy subjects (an amount of object to be assayed or amount ratio); a mean value +2SD; a mean value +3SD; and the maximum value of a judgment indicator obtained from a group of samples derived from healthy subjects (an amount of object to be assayed or amount ratio) +2SD. Especially, mean value +2SD is, for example, 21 (ng/ml) for t-CPA1, 34 (ng/ml) for PCPA1, 4.8 (ng/ml) for t-CPA2 and 4.7 (ng/ml) for PCPA2.

3-5. Effect of a Judgment Method for Pancreas Cancer of the Present Invention

Since a differential assay of CPA1 and CPA2 (or PCPA1 and PCPA2) has been thought impossible up to now, relationship between an amount of CPA1, an amount of CPA2, amount of PCPA1, amount of PCPA2, a total amount of CPA1 and PCPA1 (t-CPA1 amount) and a total amount of CPA2 and PCPA2 (t-CPA2 amount) and pancreas cancer has not been known.

Such relationship is first elucidated clearly as a result that an amount of CPA1, an amount of CPA2, an amount of PCPA1 and an amount of PCPA2 can be separately assayed by an assay method of the present invention, which can distinguish between CPA1 and CPA2 (or PCPA1 and PCPA2) and relationship between thus obtained amount (value) and pancreas cancer can be examined.

Consequently, a judgment method for pancreas cancer of the present invention is able to judge pancreas cancer, especially pancreas cancer at initial stage, i.e. stage 1 and stage 2, highly precisely and simply.

Further, since it is found that CPA level increases (shown high value) in a sample (serum) of a patient with acute pancreatitis, when CPA is used as a judgment indicator, accuracy of differential judgment of acute pancreatitis from pancreas cancer (especially early pancreas cancer at stage 1 and stage 2) may be decreased. Consequently, for distinguishing between them, PCPA, PCPA1 and PCPA2 (especially PCPA1) are preferably used as judgment indicator.

Since a method of the present invention is able to assay PCPA, PCPA1 or PCPA2 simply and easily as well as within short time as compared with the aforesaid complex and complicated conventional method for assaying PCPA activity (Peterson et al., Biochemistry, 22: 3077-3082, 1983; Published Japanese translation of PCT international publication for patent application No. 2001-518791; and Clinica Chimica Acta 292: 107-115, 2000), a differential judgment between acute pancreatitis and pancreas cancer (especially early pancreas cancer in stage 1 or stage 2) can be performed rapidly by using PCPA, PCPA1 or PCPA2 as judgment indicator.

4. A Kit of the Present Invention

4-1. An Assay Kit of the Present Invention

An assay kit of the present invention, i.e. a kit for assaying an amount of CPA1, an amount of CPA2, an amount of PCPA1, an amount of PCPA2, a total amount of CPA1 and PCPA1 (t-CPA1), a total amount of CPA2 and PCPA2 (t-CPA2), a total amount of CPA1 and CPA2 (total CPA), a total amount of PCPA1 and PCPA2 (total pro CPA), or a total amount of CPA1, CPA2, PCPA1 and PCPA2 (total t-CPA), includes, as described above, such one as consists of at least: (1) one or more antibody selected from a specific antibody of the present invention (a t-CPA1 specific antibody, a t-CPA2 specific antibody, a PCPA1 specific antibody and a PCPA2 specific antibody); (2) one or more antibody selected from a specific antibody of the present invention, and one or more antibody selected from a specific antibody of the present invention, a t-CPA common antibody and a PCPA common antibody ; (3) one or more antibody selected from a specific antibody of the present invention, and one or more antibody selected from a specific antibody of the present invention, a t-CPA common antibody and a PCPA common antibody wherein either one is a labeled antibody; (4) one or more antibody selected from a labeled specific antibody of the present invention and one or more antibody immobilized in an insoluble carrier selected from a specific antibody of the present invention, a t-CPA common antibody and a PCPA common antibody; (5) one or more labeled antibody selected from a specific antibody of the present invention, a t-CPA common antibody and a PCPA common antibody and one or more antibody selected from a specific antibody of the present invention immobilized in an insoluble carrier; and (6) one or more antibody selected from a specific antibody of the present invention, and one or more antibody selected from a specific antibody of the present invention, a t-CPA common antibody and a PCPA common antibody wherein either one antibody constitutes a conjugate bound to a separation improving substance and either one non-conjugated antibody or a conjugated antibody is at least labeled with a labeling substance. The constituent features, preferable aspects and typical examples are as described hereinabove.

In an assay kit of the present invention, a standard solution containing at least one of CPA1, CPA2, PCPA1 and PCPA2 in predetermined concentration and a manual including an assay method of the present invention and/or use in a judgment method for pancreas cancer of the present invention as explained hereinbefore may be attached.

The manual hereinabove means a user's manual for the kit, a package insert or a pamphlet (leaflet), in which features, principle, operational procedure, and the like in an assay method of the present invention, or features, principle, operational procedure, and the like in a judgment method for pancreas cancer of the present invention (e.g. use of obtained amount (value) of objects to be assayed itself as a judgment indicator of pancreas cancer or use of amount ratio (proportion) of two objects to be assayed as a judgment indicator of pancreas cancer, or a judgment method using them, etc.) are described substantially in descriptions or illustrations.

Further, the above kit can be used in combination with electrophoretic apparatus, especially capillary electrophoretic apparatus (e.g. micro fluidic device, etc.).

4-2. A Kit for Judgment of Pancreas Cancer of the Present Invention

Examples of a kit for judgment of pancreas cancer of the present invention includes, as described above, such one as consists of at least (1) one or more antibody selected from a specific antibody of the present invention (a t-CPA1 specific antibody, a t-CPA2 specific antibody, a PCPA1 specific antibody and a PCPA2 specific antibody); (2) one or more antibody selected from a specific antibody of the present invention, and one or more antibody selected from a specific antibody of the present invention, a t-CPA common antibody and a PCPA common antibody ; (3) one or more antibody selected from a specific antibody of the present invention, and one or more antibody selected from a specific antibody of the present invention, a t-CPA common antibody and a PCPA common antibody wherein either one is a labeled antibody; (4) one or more antibody selected from a labeled specific antibody of the present invention and one or more antibody, immobilized on an insoluble carrier, selected from a specific antibody of the present invention, at-CPA common antibody and a PCPA common antibody ; (5) one or more labeled antibody selected from a specific antibody of the present invention, a t-CPA common antibody and a PCPA common antibody and one or more antibody selected from a specific antibody of the present invention, immobilized on an insoluble carrier; and (6) one or more antibody selected from a specific antibody of the present invention, and one or more antibody selected from a specific antibody of the present invention, a t-CPA common antibody and a PCPA common antibody wherein either one antibody constitutes a conjugate bound to a separation improving substance and either the non-conjugated antibody or the conjugated antibody is at least labeled with a labeling substance, and the constituent features, preferable aspects and typical examples are as described above.

In a kit for judgment of pancreas cancer of the present invention, a standard solution containing at least one of CPA1, CPA2, PCPA1 and PCPA2 with predetermined concentration and a manual including use in a judgment method for pancreas cancer of the present invention as explained hereinbefore may be attached.

The manual hereinabove means a user's manual for the kit, a package insert or a pamphlet (leaflet), in which features, principle, operational procedure, and the like in a judgment method for pancreas cancer of the present invention (e.g. use of obtained amount (value) of objects to be assayed itself as a judgment indicator of pancreas cancer or use of amount ratio (proportion) of two objects to be assayed as a judgment indicator of pancreas cancer, or a judgment method using them, etc.) or/and features, principle, operational procedure, etc. in the assay method of the present invention are described substantially in descriptions or illustrations.

Further, the above kit can be used in combination with electrophoretic apparatus, especially capillary electrophoretic apparatus (e.g. micro fluidic device, etc.).

The present invention will be explained further in detail by referring to Examples, but the present invention is not construed as limiting to these Examples.

### EXAMPLES

### Example

(Expression of PCPA1 Protein and PCPA2 Protein)

(1) Preparation of a Template cDNA

A cDNA was prepared by a reverse transcription reaction with a template Total RNA Pancreas (Normal) (Lot. A40710) (Biochain Institute). Reverse transcriptase (AMV) (Nippon Gene Co.) was used to prepare a cDNA. DEPC-water (Nippon Gene Co.) of 14 μl containing 1 μg of total RNA was prepared, and a reverse transcription reaction was performed according to an attached protocol. After heat treatment of the reverse transcription reaction at 65° C. for 5 minutes, the reaction mixture was cooled on ice, mixed with 2 μl of 10× RT buffer, 1 μl of 10 μM RT primer of, 1 μl of 10 mM dNTPs, 1 μl of 0.1 M DTT, 0.5 μl of RNase inhibitor and 0.5 μl of Reverse Script II, reacted at 42° C. for 30 minutes and heated at 85° C. for 5 minutes. To thus obtained reaction mixture was added RNaseH 1 μg/μl and treated at 37° C. for 15 minutes. Thus obtained solution was used as template cDNA (pancreatic cDNA).

(2) Cloning of PCPA1 Gene and Construction of Expression Plasmid

Two types of oligonucleotides for a PCR primer (a primer for obtaining full-length base sequence) (ATGCGGGGGTTGCTGGTGTTGAGTGTCCTG: SEQ ID NO: 1) and (TCAGTAGGGGTGATTCAGGGTGTGCTCCAT: SEQ ID NO: 2) were synthesized by a conventional method based on a base sequence information of homo sapiens carboxypeptidase A1 (pancreatic) mRNA, complete cds. GenBank accession BT007313. Using these two types of primers, PCR was performed according to the attached protocol of Pfu polymerase (Straragene).

A reaction mixture of 15 μl, consisting of 1.5 μl of 10× Pfu buffer and 1.2 μl of each 2.5 mM dNTP mixture (Takara Bio Inc.), each 0.4 μl of above two types of primers prepared in concentration of 20 mM, 2 μl of a template cDNA solution, 1 μl of Pfu polymerase and 8.5 μl of sterilized purified water, was used as a sample for PCR.

The sample for PCR 15 μl was pretreated at 95° C. for 2 minutes, then reacted at 95° C. for 30 seconds, 63° C. for 30 seconds and 72° C. for 4 minutes for 42 cycles by using Thermal Cycler (Type: DNA Engine PTC225), MJ Research Inc., and further reacted at 72° C. for 2 minutes for PCR to amplify human PCPA1 full length (prepro) gene (SEQ ID NO: 5).

After PCR, A (adenine) was attached to both ends of the PCR product (obtained fragments) according to the protocol attached to a pCR4-TOPO vector (Invitrogen Corp.) by using Pfu polymerase (Straragene), and the product was separated with agarose gel electrophoresis to cut out an objective band (DNA fragment). Thus obtained DNA fragment was cloned to the PCR4-TOPO vector (Invitrogen Corp.) according to the protocol attached to PCR4-TOPO vector (Invitrogen Corp.). Accordingly, a vector having full-length sequence of PCPA1 gene (SEQ ID NO: 5) could be obtained.

E. coli DH5α was transformed by using the vector containing full length sequence of human PCPA1 gene, and thus obtained transformant was cultured, then plasmid (expressed plasmid) was recovered by using a “SV Minipreps DNA Purification System kit” (Promega Corp.).

A base sequence of the insert fragment was confirmed by using Base Station (MJ Research Inc.), and found to be identical with human PCPA1 base sequence.

PCR was conducted again using two types of oligonucleotides for a PCR primer (a tag sequence attached primer for high expression of protein) (CCGAATTCACATCATCACCATCACCATAAGGAGGACTTTGTGGGGCATCAGGTGCTC: SEQ ID NO: 3) and (CCGAATTCGCGTAGGGTGATTCAGGGTGTGCTCCATGAT: SEQ ID NO: 4) synthesized by the same way based on the identical base sequence information of above, according to the protocol attached to Pfu polymerase (Straragene) as follows.

A reaction mixture of 15 μl consisting of 1.5 μl of 10× Pfu buffer and 1.2 μl of each 2.5 mM dNTP mixture (Takara Bio Inc.), each 0.4 μl of above two types of primers prepared in concentration of 20 mM, 2 μl of a template cDNA solution, 1 μl of Pfu polymerase and 8.5 μl of sterilized purified water, was used for a sample for PCR.

The sample for PCR 15 μl was pretreated at 95° C. for 1 minutes, then reacted at 95° C. for 30 seconds, 64° C. for 30 seconds and 72° C. for 4 minutes for 25 cycles by using “Thermal Cycler” (Type: DNA Engine PTC225), MJ Research Inc., and further heated at 72° C. for 2 minutes for PCR to amplify human PCPA1 gene (the base sequence No. 48-1260 in SEQ ID NO: 5).

Thus obtained PCR fragment was digested with restriction enzyme EcoRI (Nippon Gene Co.), and the product was separated with agarose gel electrophoresis to cut out an objective band (DNA fragment).

Thus obtained DNA fragment was inserted into EcoRI site in the p3× FLAG-CMV-13 vector treated similarly with EcoRI (Nippon Gene Co.) by using a “Ligation kit ver. 2” (Takara Bio Inc.) according to the attached protocol.

E. coli DH5α was transformed by using a vector containing human PCPA1 gene (the base sequence No. 48-1260 in SEQ ID NO: 5), and thus obtained transformant was cultured, then plasmid (expressed plasmid) was recovered by using a “SV Minipreps DNA Purification System kit” (Promega Corp.).

Direction of the inserted DNA fragment sequence (human PCPA1 gene) and sequence were confirmed to have no error, by using “Base Station” (MJ Research Inc.). Accordingly, PCPA1 protein expression plasmid having the following sequence could be obtained: 6× His epitope tag gene was sequenced in the upstream of the PCPA1 gene sequence (the base sequence No. 48-1260 in SEQ ID NO: 5) and 3× FLAG epitope tag gene was sequenced in the downstream thereof.

(3) Cloning of PCPA2 Gene and Construction of Expression Plasmid

Two types of oligonucleotide for a PCR primer (a primer for obtaining full length base sequence) (ATGAGGTTGATCCTGTTTTTTGGTGCCCTT: SEQ ID NO: 7) and (CTAATAGGGGTGGTCTCGCACATGCTCCAT: SEQ ID NO: 8) were synthesized by a conventional method based on the base sequence of the homo sapiens carboxypeptidase A2 (pancreatic) mRNA, complete cds. GenBank accession BT007403. Using these two types of primers, PCR was performed by the same way as above (2), and the human PCPA2 full-length (prepro) gene (SEQ ID NO: 11) was amplified.

Subsequently, after obtaining a vector having full-length sequence of PCPA2 gene (SEQ ID NO: 11) by the same way as above (2), transformation and recovery of plasmid (expression plasmid) were performed.

A base sequence of the insert fragment was confirmed by using Base Station (MJ Research Inc.), and found to be identical with the human PCPA2 base sequence.

PCR was conducted again by the same way as above (2) using two types of oligonucleotide for a PCR primer (a tag sequence attached primer for high expression of protein) (CCGAATTCACATCATCACCATCACCATCTAGAAACATTTGTGGGAGACCAAGTTCTT: SEQ ID NO: 9) and (CCGAATTCGCATAGGGGTGGTCTCGCACATGCTCCATGAT: SEQ ID NO: 10), and the human PCPA2 gene (the base sequence No. 48-1254 in SEQ ID NO: 11) was amplified.

Then digestion with a restriction enzyme, electrophoretic treatment, cutting out of a band (DNA fragment), insertion to the vector and confirmation of the insert fragment were performed by the same way as in the above (2).

Accordingly, the PCPA2 protein expression plasmid having the following sequence could be obtained: 6× His epitope tag gene was sequenced in the upstream of the PCPA2 gene sequence (the base sequence No. 48-1254 in SEQ ID NO: 11) and 3× FLAG epitope tag gene was sequenced in the downstream thereof.

(4) Gene Transfer into Mammalian Cultured Cells

PCPA1 protein expression plasmid obtained in the above (1) was transferred into the HEK-293 cell using “Lipofectamine 2000” (Invitrogen Corp.) according to the attached protocol. Since an objective transfectant was acquired neomycin (Geneticin) resistance, Geneticin (Wako Pure Chemicals Inc.) was added in culture fluid to be final concentration of 0.75 mg/ml after 24 hours of transfection, and Geneticin selective culture was performed for about 2 weeks. Then cloning of Geneticin resistant strain was performed by limiting dilution to obtain Geneticin resistant strain 105 clones, to which PCPA1 protein expression plasmid was transfected.

Using PCPA2 protein expression plasmid obtained in the above (2) and performing cloning by the same way as above, Geneticin resistant strain 120 clones, to which the PCPA2 protein expression plasmid was transfected, could be obtained.

(5) Selection of PCPA1 Protein High Expression Strain and PCPA2 Protein High Expression Strain

Geneticin resistant strain 105 clones, to which PCPA1 protein expression plasmid obtained in the above (4) was transfected, were cultured in “Opti-MEM medium” (Gibco), and the culture supernatant (serum-free medium) each 200 μl was used as a sample, then dot Western blotting was conducted by using an anti-FLAG antibody (Sigma-Aldrich Co.) as the primary antibody, AP (alkaline phosphatase) labeled anti-mouse IgG goat (Sigma-Aldrich Co.) as the secondary antibody and an AP coloring kit (Bio-Rad Laboratories) as a detection reagent to select two clones of the Geneticin resistant strain, to which PCPA1 protein expression plasmid was transfected with strongly confirmed coloring.

Using Geneticin resistant strain 120 clones, to which PCPA2 protein expression plasmid obtained in the above (4) was transfected, similar confirmation to the above was performed to select two clones of Geneticin resistant strain, to which PCPA2 protein expression plasmid was transfected.

After selected four clones were cultured in “Opti-MEM medium” (Gibco), a ten times concentrated solution of the culture supernatant treated with ultrafiltration, each 20 μl, was used as a sample. After the sample was treated with SDS-PAGE, dot Western blotting was conducted by using an anti-FLAG antibody (Sigma-Aldrich Co.) as the primary antibody, AP (alkaline phosphatase) labeled anti-mouse IgG goat (Sigma-Aldrich Co.) as the secondary antibody and an “AP coloring kit” (Bio-Rad Laboratories) as a detection reagent, then the expression of PCPA1 protein and PCPA2 protein was confirmed.

Namely, in the above (2), since 6× His gene (SEQ ID NO: 3) was sequenced in the upstream of the sequence of PCPA1 gene (base sequence No. 48-1260 in SEQ ID NO: 5) and 3× FLAG gene (SEQ ID NO: 4) was sequenced in the downstream, continued from PCPA1 gene sequence, PCPA1 was expressed as the fused protein (6× His-proCPA A1-3× FLAG) which bound to 6× His tag sequence and 3× FLAG sequence.

Further, in the above (3), since 6× His gene (SEQ ID NO: 9) was sequenced in the upstream of the sequence of PCPA2 gene (base sequence No. 48-1254 in SEQ ID NO: 11) and 3× FLAG gene (SEQ ID NO: 10) was sequenced in the downstream, continued from PCPA2 gene sequence, PCPA2 was expressed as the fused protein (6× His-proCPA A2-3× FLAG) which bound to 6× His tag sequence and 3× FLAG sequence.

Consequently, if expression of 3× FLAG sequence and 6× His sequence is confirmed, expression of PCPA1 gene sequence or PCPA2 gene sequence in the upstream and the downstream regions can be said to be confirmed also.

As a control, the p3× FLAG-CMV-13 vector (Sigma-Aldrich) without having inserted PCPA1 protein gene sequence (a base sequence No. 48-1260 in SEQ ID NO: 5) or PCPA2 protein gene sequence (a base sequence No. 48-1254 in SEQ ID NO: 11) was transfected into HEK-293 cells, according to the method of above (4) to obtain Geneticin resistant cells. A ten times concentrated solution of the culture supernatant of the Geneticin resistant cell culture 20 μl was similarly subjected to electrophoresis and Western blotting analysis.

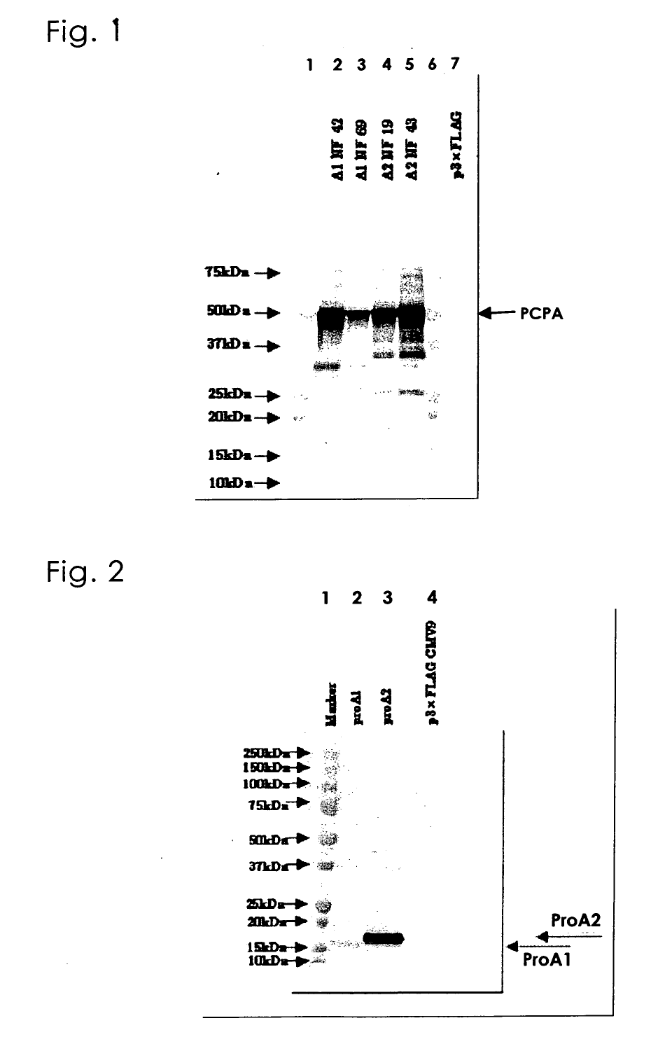

Result is shown in FIG. 1. In FIG. 1, lanes 2 and 3 show the results using 20 μl of ten times concentration of the culture supernatant of two clones, which are high expression and stable strains transfected with PCPA1 protein expression plasmid; and lanes 4 and 5 show the results using 20 μl of a ten times concentrated solution of the culture supernatant of two clones, which are high expression and stable strains transfected with PCPA2 protein expression plasmid. Further, lane 7 shows the result (control) using 20 μl of a ten times concentrated solution of the culture supernatant of the Geneticin resistant cell culture, which was obtained by transfection of the p3× FLAG-CMV-13 vector (Sigma-Aldrich) without having inserted PCPA1 protein gene sequence (the base sequence No. 48-1260 in SEQ ID NO: 5) or PCPA2 protein gene sequence (a base sequence No. 48-1254 in SEQ ID NO: 11) into HEK-293 cells according to the method of above (4). Lanes 1 and 6 show Precision Plus Protein Standards (Bio-Rad Laboratories).

Further, “←” indicates PCPA1 protein (50 kDa) and PCPA2 protein (50 kDa).

As apparent from FIG. 1, among the two clones of high expression and stable strain transfected with PCPA1 protein expression plasmid, a band was strongly confirmed in the clone A1 NF42 (lane No. 2), and among the two clones of high expression and stable strain transfected with PCPA2 protein expression plasmid, a band was strongly confirmed in the clone A2 NF43 (lane No. 5). In the lane 7 (control), which was the Genetic in resistant strain using the p3× FLAG-CMV-13 vector without having inserted PCPA1 protein gene sequence (the base sequence No. 48-1260 in SEQ ID NO: 5) or PCPA2 protein gene sequence (the base sequence No. 48-1254 in SEQ ID NO: 11), no band was recognized.

As described hereinabove, using the HEK-293 cell, cell strain expressing an objective PCPA1 protein (hereinafter designates as recombinant PCPA1 protein) and cell strain expressing an objective PCPA2 protein (hereinafter designates as recombinant PCPA2 protein) could be obtained.

### Example 2

(Preparation of an Anti-PCPA1 Monoclonal Antibody and an Anti-PCPA2 Monoclonal Antibody)

(1) Preparation of an Immunogen (Recombinant PCPA1 Protein and Recombinant PCPA2 Protein)

Recombinant PCPA1 protein expressing the HEK-293 cell strain obtained in Example 1 was cultured in “D-MEM medium” (Gibco, containing 10% bovine serum albumin (BSA)) using 20 pieces of a 500 cm² flask to 100% confluent state, then the medium was replaced to “Opti-MEM medium” (Gibco, serum-free). Culture was continued for 5 days, and the culture supernatant was recovered and collected.

The culture supernatant of 1 L obtained by the above culture was centrifuged at 3000 rpm for 30 minutes to obtain an expressed protein fraction in the supernatant. Thus obtained protein fraction was subjected to affinity chromatography according to the protocol attached to “ANTI-FLAG M2 Affinity Gel Freezer-Safe” (Sigma-Aldrich), and objective protein was purified to obtain a fraction containing recombinant PCPA1 protein.

Using the HEK-293 cell strain expressing recombinant PCPA2 protein obtained in Example 1, a fraction containing recombinant PCPA2 protein was obtained by the same way as above.

(2) Preparation of an Anti-PCPA1 Monoclonal Antibody and an Anti-PCPA2 Monoclonal Antibody

Recombinant PCPA1 protein obtained in the above (1) was mixed with “Adjuvant Complete Freund” (Wako Pure Chemical Industry Co.), and the mixture was administered intraperitoneally into BALB/c mice (female), 3 μg/mouse, for the 1st immunization. After two weeks from the 1st immunization, recombinant PCPA1 protein was mixed with “Adjuvant Complete Freund” (Wako Pure Chemical Industry Co.), and the mixture was administered intraperitoneally into BALB/c mice (female), 10 μg/mouse, for the 2nd immunization, then further after two weeks, the mixture was intraperitoneally administered by the same way as in the 2nd immunization to perform the 3rd immunization. After one month from the 3rd immunization, recombinant PCPA1 protein was mixed with physiological saline, and the mixture was administered into BALB/c mice (female), 20 μg/mouse, for the final immunization.

On day 3 after the final immunization, spleen was excised. Spleen was dispersed on a sterilized stainless steel mesh, suspended in “Daigo T medium” (Wako Pure Chemicals Industry Co.), and washed by repeated centrifugation. Collected spleen cells, 1.5×10⁸ cells, and mouse myeloma cells (P3X63Ag8U1), 3×10⁷ cells, which were washed by the same way as in spleen cells in the “Daigo T medium”, were mixed in “Daigo T medium”, and centrifuged at 1450 rpm for 10 minutes.

To thus obtained precipitate, 50% polyethylene glycol 1540, 1 ml, was gradually added with stirring at 37° C. for 1 minute to make cell fusion, then “Daigo T medium” of 9 ml at 37° C. was further added with stirring for 5 minutes, and centrifuged at 1450 rpm for 10 minutes. Thus obtained fused cells were suspended in “HAT medium” of 60 ml, separately added 0.2 ml into a 96 well microplate, and incubation was continued with exchanging “HAT medium” at intervals of 2 to 3 days. After 8 to 10 days from cell fusion, the culture supernatant was collected, and screening of antibody activity of an anti-PCPA1 antibody was performed by using ELISA, and cells with strong antibody activity in the well were subjected to cloning by applying limiting dilution. After 8 days from cloning, the culture supernatant of one colony/well was collected, and screening of antibody activity of an anti-PCPA1 antibody was performed by using ELISA to obtain a monoclonal antibody producing hybridoma.

Among these clones, an anti-PCPA1 antibody of 18 clones could be selected.

Using recombinant PCPA2 protein obtained in the above (1), immunization and cloning were performed by the same way as in the above to obtain a monoclonal antibody producing hybridoma, and an anti-PCPA2 antibody of 16 clones could be selected.

### Example 3

(Evaluation of Specificity of an Obtained Monoclonal Antibody)

(1) Obtaining Pro-Region Protein

To find a binding site of thus obtained antibodies, PCPA1 pro-area (pro-region: PCPA1 specific region) (amino acid sequence of No. 16-110 in SEQ ID NO: 6) protein, and PCPA2 pro-area (pro-region: PCPA2 specific region) (amino acid sequence of No. 16-110 in SEQ ID NO: 12) protein were attempted to obtain.

A vector containing human PCPA1 full-length gene obtained in Example 1, (2) was used as a template DNA. Using two types of oligonucleotides (CCGAATTCACATCATCACCATCACCATAAGGAGGACTTTGTGGGGCATCAGGTGCTC: SEQ ID NO: 3) and (CCGAATTCTCACCGGGACCGGAAGGCGAACATCTGCTCCTG: SEQ ID NO: 13), PCR was conducted according to a method in Example 1, (2) to amplify sequence (the base sequence 48-330 bp in SEQ ID NO: 5) of pro-area (pro-region) of human PCPA1.

After the amplified fragment was confirmed to be identical with the base sequence (the base sequence 48-330 bp in SEQ ID NO: 5) of pro-area (pro-region) of human PCPA1, digestion with a restriction enzyme, electrophoretic treatment and cutting off a band (DNA fragment) were performed by the same way as in Example 1, (2), and a DNA fragment thus obtained was inserted into the p3× FLAG-CMV9 vector (Sigma-Aldrich) to obtain PCPA1 pro-region protein expression plasmid.

Thus obtained PCPA1 pro-region protein expression plasmid was transfected into the HEK-293 cells by the same means in Example 1, (4) to obtain cells transfected with PCPA1 pro-region protein expression plasmid (the HEK-293 cell strain expressing PCPA1 pro-region protein).

After culturing Thus obtained PCPA1 pro-region protein expressing HEK-293 cell strain by the same way as in Example 2, (1), the culture supernatant containing PCPA1 pro-region protein was obtained by transient expression.

The culture supernatant was subjected to affinity chromatography according to the protocol attached to “ANTI-FLAG M2 Affinity Gel Freezer-Safe” (Sigma-Aldrich) and objective protein was purified, then PCPA1 pro-region protein was obtained.

In addition, PCPA2 pro-region protein was obtained by the same way as above as follows.

PCR was conducted by the same method as above except for using two types of oligonucleotides (CCGAATTCACATCATCACCATCACCATCTAGAAACATTTGTGGGAGACCAAGTTCTT: SEQ ID NO: 9) and (CCGAATTCTCACCTATTAAAAAGCATTTCTTCATTCTCTTT: SEQ ID NO: 14), and a sequence (the base sequence 48-330 bp in SEQ ID NO: 11) of pro-area (pro-region) of human PCPA2 was amplified. After the amplified fragment was confirmed to be identical with the base sequence (the base sequence 48-330 bp in SEQ ID NO: 11) of pro-area (pro-region), digestion with a restriction enzyme, electrophoretic treatment and cutting off a band (DNA fragment) were performed by the same way as above, and a DNA fragment thus obtained was inserted into the p3× FLAG-CMV9 vector (Sigma-Aldrich) to obtain PCPA2 pro-region protein expression plasmid. Thus obtained PCPA2 pro-region protein expression plasmid was transfected into the HEK293 cells by the same means as above to obtain the HEK-293 cell strain expressing PCPA2 pro-region protein. After culturing thus obtained PCPA2 pro-region protein expressing HEK-293 cell strain by the same way as above, the culture supernatant containing PCPA2 pro-region protein was obtained by transient expression. The culture supernatant was purified by using affinity chromatography by the same way as above to obtain PCPA2 pro-region protein.

Using thus obtained cells transfected with PCPA1 pro-region protein expression plasmid (the HEK-293 cell strain expressing PCPA1 pro-region protein) and cells transfected with PCPA2 pro-region protein expression plasmid (the HEK-293 cell strain expressing PCPA2 pro-region protein), culturing, ultrafiltration of the culture supernatant, SDS-PAGE and Western blotting were performed by the same way as in Example 1, (5), and expression of PCPA1 pro-region protein and expression of PCPA2 pro-region protein were confirmed. As a control, the p3× FLAG-CMV9 vector (Sigma-Aldrich) without having inserted base sequence of PCPA1 pro-area (pro-region) (the base sequence of 48-330 bp in SEQ ID NO: 5) or the base sequence of PCPA2 pro-area (pro-region) (the base sequence of 48-330 bp in SEQ ID NO: 11) was transfected into HEK-293 cells by the same method as above, and the culture supernatant of thus obtained cells were used.

Result is shown in FIG. 2. In FIG. 2, lane 2 shows the result using 20 μl of a ten times concentrated solution of the culture supernatant of the HEK-293 cell strain expressing PCPA1 pro-region protein and lane 3 shows the result using 20 μl of a ten times concentrated solution of the culture supernatant of the HEK-293 cell strain expressing PCPA2 pro-region protein. Further, lane 4 shows the result (control) using 20 μl of ten times concentrated solution of the culture supernatant of the HEK-293 cell strain, to which the p3× FLAG-CMV9 vector without having inserted base sequence of PCPA1 pro-region (the base sequence of 48-330 bp in SEQ ID NO: 5) or the base sequence of PCPA2 pro-region (the base sequence of 48-330 bp in SEQ ID NO: 11) was transfected. Lane 1 shows a molecular weight marker (Bio-Rad Laboratories, Precision Plus Protein Standards).

Further, “←Pro A1” indicates PCPA1 pro-region protein (16 kDa) and “←Pro A2” indicates PCPA2 pro-region protein (17 kDa).

As apparent from FIG. 2, expression of PCPA1 pro-region protein was confirmed in the Geneticin resistant strain (lane No. 2), to which PCPA1 pro-region protein expression plasmid was transfected, and expression of PCPA2 pro-region protein was confirmed in the Geneticin resistant strain (lane No. 3), to which PCPA2 pro-region protein expression plasmid was transfected. No band was recognized in lane No. 4 (control) of the Geneticin resistant strain using the p3× FLAG-CMV9 vector without having inserted the base sequence of PCPA1 pro-area (pro-region) (the base sequence of 48-330 bp in SEQ ID NO: 5) or the base sequence of PCPA2 pro-area (pro-region) (the base sequence of 48-330 bp in SEQ ID NO: 11).

As described hereinabove, using the HEK-293 cell, cell strain expressing objective PCPA1 pro-region protein (hereinafter designates as recombinant PCPA1 pro-region protein) and cell strain expressing PCPA2 pro-region protein (hereinafter designates as recombinant PCPA2 pro-region protein) could be obtained.

(2) Evaluation of Antibody Specificity by EIA

Antibody specificity of an anti-PCPA1 monoclonal antibody (18 clones) and an anti-PCPA2 monoclonal antibody (16 clones) obtained in Example 2 was evaluated by EIA with recombinant PCPA1 pro-region protein and recombinant PCPA2 pro-region protein prepared in concentrations of 0 ng/ml, 0.1 ng/ml, 1 ng/ml and 10 ng/ml as an antigen using an EIA plate of “Nunc Immobilizer Amino” (Nunc) according to the attached protocol.

An anti-PCPA1 monoclonal antibody (18 clones) and an anti-PCPA2 monoclonal antibody (16 clones) obtained in Example 2 were used as the primary antibody, and HRP labeled anti-mouse IgG goat (Sigma-Aldrich) was used as the secondary antibody (labeled antibody). An OPD tablet (o-phenylenediamine dihydrochloride 13 mg/tablet, Wako Pure Chemicals Industry Co.) was used for detection, and after 10 minutes from reaction initiation, the reaction was terminated according to the attached protocol, and absorbancy of the reaction mixture was measured.

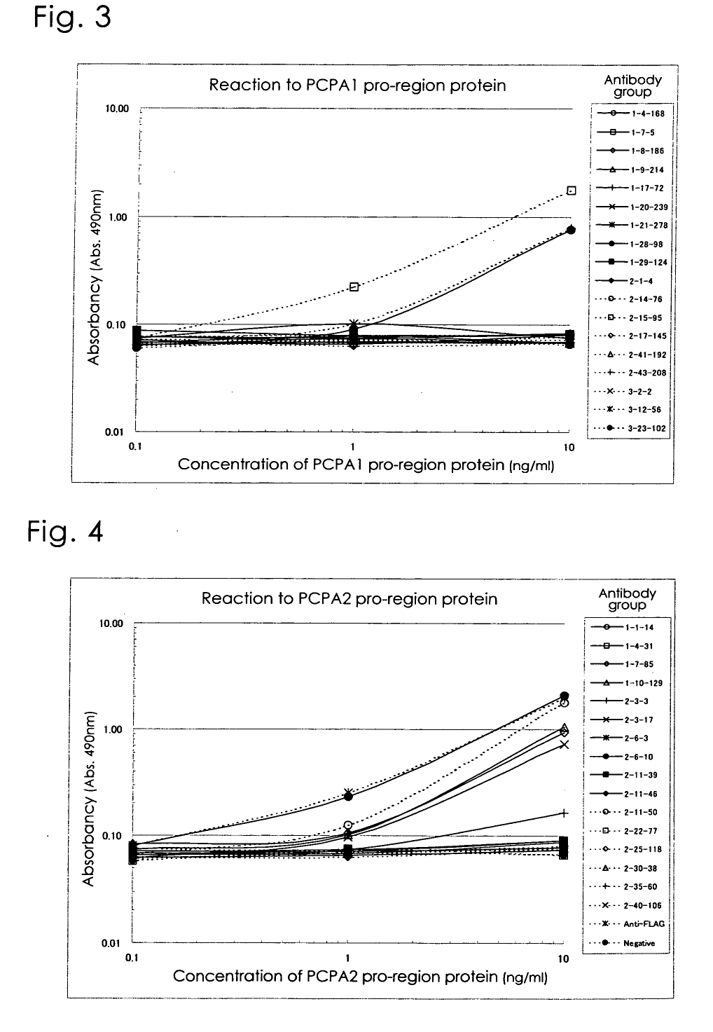

Results using an anti-PCPA1 monoclonal antibody (18 clones) were shown in FIG. 3, and results using an anti-PCPA2 monoclonal antibody (16 clones) were shown in FIG. 4.

In addition, result using an Anti-FLAG antibody (ANTI-FLAG M2 Monoclonal Antibody Unconjugated, Sigma-Aldrich) as the primary antibody in place of the above primary antibody and performing the assay by the same way as the above, and result without using an antigen (recombinant PCPA1 pro-region protein and recombinant PCPA2 pro-region protein) and performing the assay by the same way as the above are set as a control.

In FIG. 3, -∘-: result using clone No. 1-4-168; -□-: result using clone No. 1-7-5; -⋄-: result using clone No. 1-8-186; -Δ-: result using clone No. 1-9-214; -+-: result using clone No. 1-17-72; -×-: result using clone No. 1-20-239; - [symbol] -: result using clone No. 1-21-278; -●-: result using clone No. 1-28-98; -▪-: result using clone No. 1-29-124; -♦-: result using clone No. 2-1-4; ---∘---: result using clone No. 2-14-76; ---□---: result using clone No. 2-15-95; ---⋄---: result using clone No. 2-17-145; ---Δ---: result using clone No. 2-41-192; ---+---: result using clone No. 2-43-208; ---×---: result using clone No. 3-2-2; --- [symbol] ---: result using clone No. 3-12-56; and ---●---: result using clone No. 3-23-102 are shown.

In FIG. 4, -∘-: result using clone No. 1-1-14; -□-: result using clone No. 1-4-31; -⋄-: result using clone No. 1-7-85; -Δ-: result using clone No. 1-10-129; -+-: result using clone No. 2-3-3; -×-: result using clone No. 2-3-17; - [symbol] -: result using clone No. 2-6-3; -●-: result using clone No. 2-6-10; -▪-: result using clone No. 2-11-39; -♦-: result using clone No. 2-11-46; ---∘---: result using clone No. 2-11-50; ---□---: result using clone No. 2-22-77; ---⋄---: result using clone No. 2-25-118; ---Δ---: result using clone No. 2-30-38; ---+---: result using clone No. 2-35-60; ---×---: result using clone No. 2-40-106; --- [symbol] ---: result using anti-FLAG antibody as the primary antibody (control); and ---●---: a result without using antigen (control) are shown.

As apparent from FIG. 3, it could be confirmed that among 18 clones of anti-PCPA1 monoclonal antibodies obtained in Example 2, three clones (clone No. 2-15-95, clone No. 2-43-208 and clone No. 1-28-98) were monoclonal antibodies specifically recognizing the pro-area of PCPA1 (PCPA1 pro-region protein: PCPA1 specific region protein) (amino acid sequence of No. 16-110 in SEQ ID NO: 6) (a PCPA1 specific monoclonal antibody), and 15 clones other than that were monoclonal antibodies recognizing constant region existing commonly in both of CPA1 and PCPA1 (a t-CPA1 specific monoclonal antibody).

Clone No. 2-15-95 was selected from these clones at random as hybridoma producing a PCPA1 specific monoclonal antibody and was named as hybridoma CPA15-95 which produced the monoclonal antibody2-15-95. Further, clone No. 1-7-5 and clone No. 2-14-76 were selected at random as hybridoma producing a t-CPA1 specific monoclonal antibody and were named as hybridoma CPA7-5 which produced the monoclonal antibody 1-7-15, and hybridoma CPA14-76 which produced the monoclonal antibody 2-14-76.

As apparent from FIG. 4, it could be confirmed that among 16 clones of anti-PCPA2 monoclonal antibodies obtained in Example 2, five clones (clone No. 2-6-10, clone No. 2-11-50, clone No. 1-10-129, clone No. 1-7-85 and clone No. 2-3-17) were monoclonal antibodies specifically recognizing pro-area of PCPA2 (PCPA2 pro-region protein: PCPA2 specific region protein) (amino acid sequence of No. 16-110 in SEQ ID NO: 12) (a PCPA2 specific monoclonal antibody), and 11 clones other than those were monoclonal antibodies recognizing constant region existing commonly in both of CPA2 and PCPA2 (a t-CPA2 specific monoclonal antibody).

Clone No. 2-11-50 was selected from these clones at random as hybridoma producing an anti-PCPA2 specific monoclonal antibody and was named as hybridoma CPA11-50 which produced the monoclonal antibody 2-11-50. Further, clone No. 2-25-118 and clone No. 1-1-14 were selected at random as hybridoma producing a t-CPA2 specific monoclonal antibody and were named as hybridoma CPA25-118 which produced the monoclonal antibody 2-25-118, and hybridoma CPA1-14 which produced the monoclonal antibody 1-1-14.

Each hybridoma was deposited at AIST Tsukuba Central 6, 1-1, Higashi 1-chome, Tsukuba-shi, Ibaraki-ken, 305-8566 Japan, International Patent Organism Depositary National Institute of Advanced Industrial Science and Technology on Sep. 16, 2004 with the following deposit numbers.
Hybridoma CA15-95: FERM ABP-10130
Hybridoma CA7-5: FERM ABP-10128
Hybridoma CA14-76: FERM ABP-10129

### Example 4

(Assay of PCPA1, t-CPA1, PCPA2 and t-CPA2 in Serum of Patients with Pancreas Cancer, Patients with Chronic Pancreatitis and Healthy Subjects)

(1) Samples

Following serum were used as samples.
Serum of healthy subjects: 26 samples
Serum of patients with chronic pancreatitis: 20 samples
Serum of patients with pancreas cancer (stage 1): 9 samples
Serum of patients with pancreas cancer (stage 2): 32 samples
Serum of patients with pancreas cancer (stage 3): 18 samples
Serum of patients with pancreas cancer (stage 4): 12 samples
   (2) Preparation of Reagents
   A Micro Plate Immobilized with a t-CPA1 Specific Monoclonal Antibody

A solution, 10 μg/ml, of the t-CPA1 specific monoclonal antibody CPA1-7-5, an antibody specifically binding to CPA1 and PCPA1, was added to a 96 wells micro plate (Corning Inc.), each 100 μl/well, reacted at 4° C. overnight, and was blocked a non-binding area by adding 20% NP-2000 (K.K. Nippi) to obtain a micro plate immobilized with t-CPA1 specific monoclonal antibody CPA1-7-5.

A Micro Plate Immobilized with a t-CPA2 Specific Monoclonal Antibody

A solution, 10 μg/ml, of the t-CPA2 specific monoclonal antibody CPA1-1-14, an antibody specifically binding to CPA2 and PCPA2, was added to a 96 wells micro plate (Corning Inc.), each 100 μl/well, reacted at 4° C. overnight, and was blocked a non-binding area by adding 20% NP-2000 (K.K. Nippi) to obtain a micro plate immobilized with t-CPA2 specific monoclonal antibody CPA1-1-14.

A POD Labeled PCPA1 Specific Monoclonal Antibody

A procedure was performed according to a conventional method described in “Enzyme Immunoassay”, Ishikawa, E. et al. Ed., 2nd Ed., Igaku-Shoin, 1982, etc. Namely, the PCPA1 specific monoclonal antibody CPA2-15-95, an antibody specifically biding to PCPA1, was reacted with 10 molar equivalent amounts of N-succinimidyl 3-[2-pyridyldithio]propionate in a phosphate buffer saline solution (PBS). The reaction mixture was subjected to gel filtration by using Sephadex G-25 to remove residual N-succinimidyl 3-[2-pyridyldithio]propionate, then reduced using DTT to reduce a SH group. Separately, POD was reacted with 10 molar equivalent amounts of m-maleimidobenzyl-N-hydroxysuccinimide ester in PBS, thereafter the residual m-maleimidobenzyl-N-hydroxysuccinimide ester was removed off by Sephadex G-25 gel filtration to obtain m-maleimidobenzyl-N-hydroxysuccinimide introduced POD. An antibody having a reduced SH group and an m-maleimidobenzyl-N-hydroxysuccinimide introduced POD were reacted to obtain the POD labeled PCPA1 specific monoclonal antibody CPA2-15-95.

A POD Labeled t-CPA1 Specific Monoclonal Antibody

Using the t-CPA1 specific monoclonal antibody CPA2-14-76, an antibody specifically binding to CPA1 and PCPA1, the POD labeled t-CPA1 specific monoclonal antibody CPA2-14-76 was obtained by the same way as above.

A POD Labeled PCPA2 Specific Monoclonal Antibody

Using the PCPA2 specific monoclonal antibody CPA2-11-50, an antibody specifically binding to PCPA2, the POD labeled PCPA2 specific monoclonal antibody CPA2-11-50 was obtained by the same way as above.

A POD Labeled t-CPA2 Specific Monoclonal Antibody

Using the t-CPA2 specific monoclonal antibody CPA2-25-118, an antibody specifically binding to CPA2 and PCPA2, the POD labeled t-CPA2 specific monoclonal antibody CPA2-25-118 was obtained by the same way as above.

(3) An Assay Method

Assay of t-CPA1 (CPA1 and PCPA1)

An assay method was a coloring method using known ELISA as follows.

To each well of a micro plate immobilized with the t-CPA1 specific monoclonal antibody CPA1-7-5 obtained in the above was added a sample of 50 μl and incubated at room temperature for 2 hours. After washing with PBS, the POD labeled t-CPA1 specific monoclonal antibody CPA2-14-76, 50 μl, obtained above was added and incubated at room temperature for 2 hours. After the reaction, the microplate was washed with PBS, and an OPD solution (o-phenylenediamine dihydrochloride, 13 mg/Tab., Wako Pure Chemical Industry Co.), 50 μl, was added, then the reaction was terminated after 10 minutes of initiation of the reaction according to the attached protocol, and absorbancy of the reaction mixture was measured using “SAFIRE” (TECAN Co.).

A calibration curve used for obtaining an amount of t-CPA1 (total amount of CPA1 and PCPA1) in a sample from the above obtained absorbancy was prepared as follows.

The t-CPA1 specific monoclonal antibody CPA1-7-5 was immobilized to “NHS-activated Sepharose 4 Fast Flow” (Amersham Bioscience K.K.) according to the attached protocol.

Human PCPA1 gene (the base sequence of 48-1260 bp in SEQ ID NO: 5) was amplified by PCR using primers of SEQ ID NO: 13 and SEQ ID NO: 2 by the same method as in Example 1. Thereafter, digestion with a restriction enzyme, electrophoretic treatment, cutting out a band (DNA fragment), insertion into a vector and confirmation of the insert were performed by the same way as in Example 1.

As a result of this operation, PCPA1 protein expression plasmid having PCPA1 gene (the base sequence of 48-1260 bp in SEQ ID NO: 5) could be obtained.

PCPA1 protein expression plasmid was transfected into the HEK-293 cells by the same way as in Example 1, and the HEK-293 cell strain with transfected PCPA1 protein expression plasmid (a stable expression strain) was cultured. PCPA1 protein (amino acid sequence No. 16-419 in SEQ ID NO: 6) (recombinant PCPA1) was obtained from thus obtained culture supernatant using “NHS-activated Sepharose 4 Fast Flow”, to which the t-CPA1 specific monoclonal antibody CPA1-7-5 was immobilized, and was used for an antigen for a calibration curve.

Thus obtained antigen for the calibration curve was prepared in concentrations of 0.4 ng/ml, 1.5 ng/ml, 12 ng/ml, 50 ng/ml and 100 ng/ml. Absorbance was measured using these samples and the same reagents and apparatus as above to prepare a calibration curve by plotting absorbancy in the ordinate and antigen concentration (ng/ml) in the abscissa.

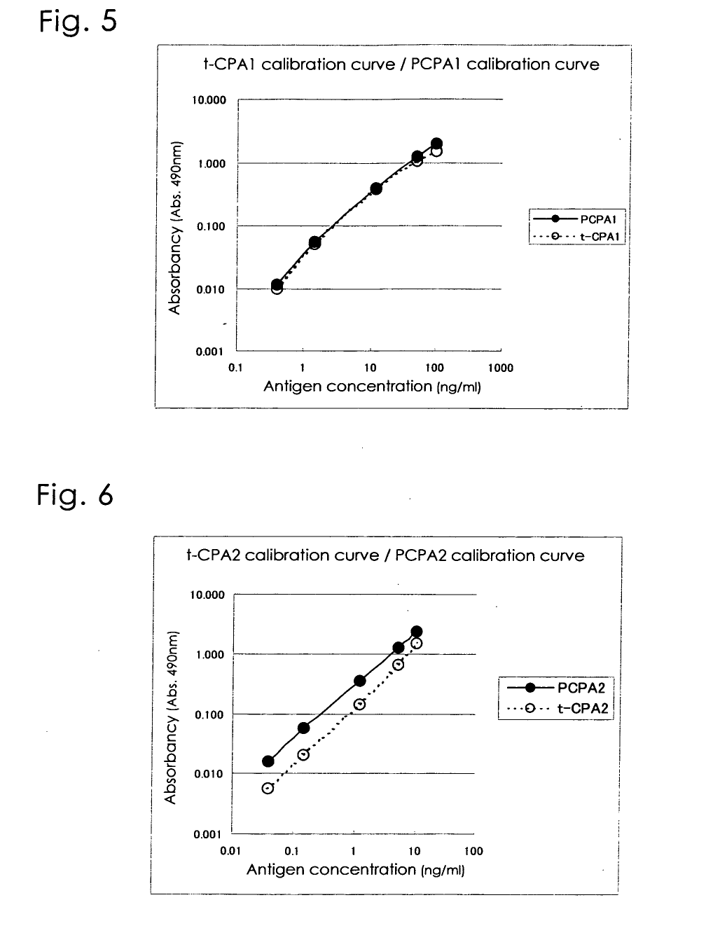

Thus obtained calibration curve is shown in FIG. 5.

Using the calibration curve, amounts of t-CPA1 in serum of patients with pancreas cancer, patients with chronic pancreatitis and healthy subjects were obtained.

Assay of PCPA1

Absorbancy was measured by the same operation using the same reagents and apparatus as in the above assay of t-CPA1, except that the POD labeled PCPA1 specific monoclonal antibody CPA2-15-95 was used in place of the POD labeled t-CPA1 specific monoclonal antibody CPA2-14-76.

A calibration curve used for obtaining amount of PCPA1 in a sample from thus obtained absorbancy was prepared by using the same antigen as the antigen for the calibration curve (recombinant PCPA1 protein) used in preparing the above calibration curve for t-CPA1 (CPA1 and PCPA1) and using the same reagents and apparatus of the above.

Thus obtained calibration curve is shown in FIG. 5.

In FIG. 5, ---∘--- indicates a calibration curve for assaying t-CPA1, and ---●--- indicates a calibration curve for assaying PCPA1.

Using the calibration curve, amounts of PCPA1 in serum of patients with pancreas cancer, patients with chronic pancreatitis and healthy subjects were obtained.

Assay of t-CPA2 (CPA2 and PCPA2)

Absorbancy was measured by the same operation using the same reagents and apparatus as in the above assay of t-CPA1, except that a micro plate immobilized with the t-CPA2 specific monoclonal antibody CPA1-1-14 is used in place of a micro plate immobilized with the t-CPA1 specific monoclonal antibody CPA1-7-5, and the POD labeled t-CPA2 specific monoclonal antibody CPA2-25-118 is used in place of the POD labeled t-CPA1 specific monoclonal antibody CPA2-14-76.

A calibration curve used for obtaining an amount of t-CPA2 (total amount of CPA2 and PCPA2) in a sample from the above-obtained absorbancy was prepared as follows.

The t-CPA2 specific monoclonal antibody CPA2-25-118 was immobilized to “NHS-activated Sepharose 4 Fast Flow” (Amersham Bioscience K.K.) according to the attached protocol.

Human PCPA2 gene (the base sequence of 48-1254 bp in SEQ ID NO: 11) was amplified by PCR using primers of SEQ ID NO: 14 and SEQ ID NO: 8, by the same method as in Example 1. Thereafter, digestion with a restriction enzyme, electrophoretic treatment, cutting out a band (DNA fragment), insertion into a vector and confirmation of the insert were performed by the same way as in Example 1.

As a result of this operation, PCPA2 protein expression plasmid having PCPA2 gene (the base sequence of 48-1254 bp in SEQ ID NO: 11) could be obtained.

PCPA2 protein expression plasmid was transfected into the HEK-293 cells by the same way as in Example 1, and the HEK-293 cell strain with transfected PCPA2 protein expression plasmid (stable expression strain) was cultured. The PCPA2 protein (amino acid sequence No. 16-417 in SEQ ID NO: 12) (recombinant PCPA2) was obtained from thus obtained culture supernatant by using “NHS-activated Sepharose 4 Fast Flow”, to which the t-CPA2 specific monoclonal antibody CPA2-25-118 was immobilized, and was used for an antigen for a calibration curve.

Thus obtained antigen for a calibration curve was prepared in concentrations of 0.04 ng/ml, 0.15 ng/ml, 1.2 ng/ml, 5 ng/ml and 10 ng/ml. Absorbance was measured using these samples and the same reagents and apparatus as above to prepare a calibration curve by plotting absorbancy in the ordinate and antigen concentration (ng/ml) in the abscissa.

Thus obtained calibration curve is shown in FIG. 6.

Using the calibration curve, amounts of st-CPA2 in serum of patients with pancreas cancer, patients with chronic pancreatitis and healthy subjects were obtained.

Assay of PCPA2

Absorbancy was measured by the same operation using the same reagents and apparatus in the above assay of t-CPA2, except that the POD labeled PCPA2 specific monoclonal antibody CPA2-11-50 was used in place of the POD labeled t-CPA1 specific monoclonal antibody CPA2-25-118.

A calibration curve used for obtaining an amount of PCPA2 in a sample from thus obtained absorbancy was prepared using the same antigen as the antigen for a calibration curve (recombinant PCPA2 protein) used in preparing the above calibration curve for t-CPA2 (CPA2 and PCPA2) and using the same reagents and apparatus of the above.

Thus obtained calibration curve is shown in FIG. 6.

In FIG. 6, ---∘--- indicates a calibration curve for assaying t-CPA2, and ---●--- indicates a calibration curve for assaying PCPA2.

Using the calibration curve, amounts of serum PCPA2 in patients with pancreas cancer, patients with chronic pancreatitis and healthy subjects were obtained.

(4) Results

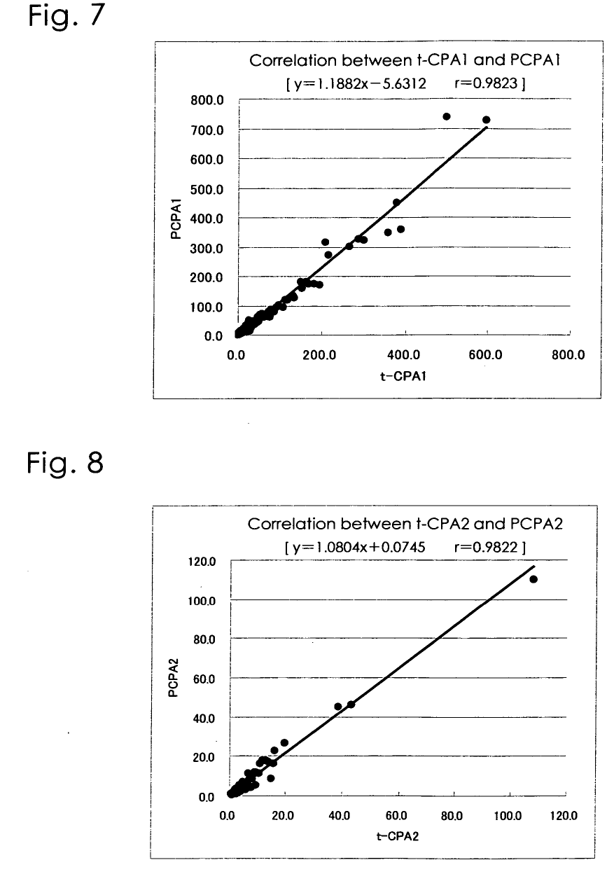

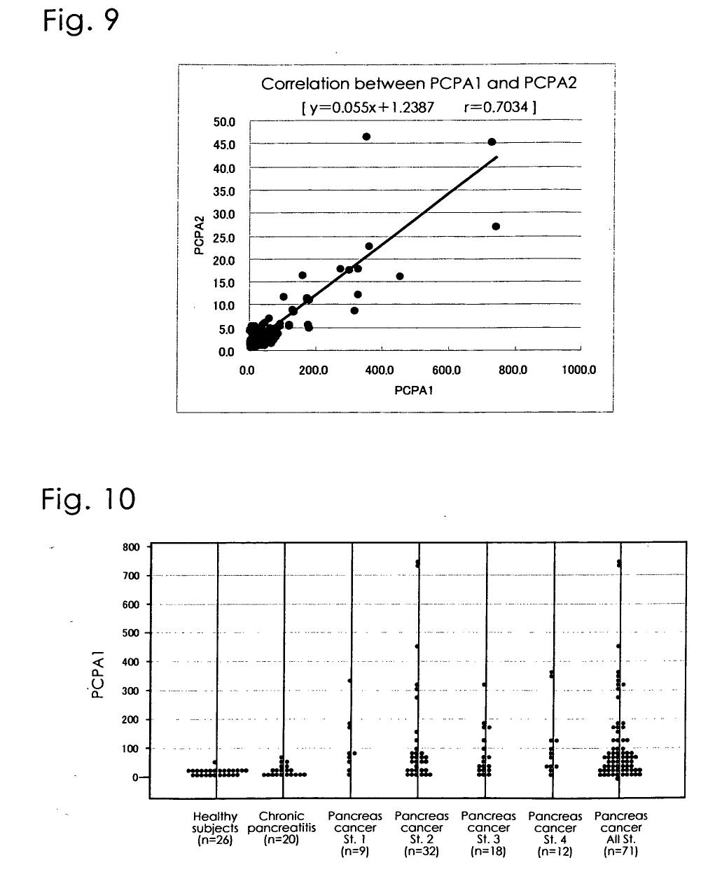

Correlation formula and correlation diagram of an amount of PCPA1 and an amount of t-PCA1 in each sample obtained above are shown in FIG. 7; correlation formula and correlation diagram of an amount of PCPA2 and an amount of t-PCA2 in each sample obtained above are shown in FIG. 8; and correlation formula and correlation diagram of an amount of PCPA1 and an amount of PPCA2 in each sample obtained above are shown in FIG. 9.

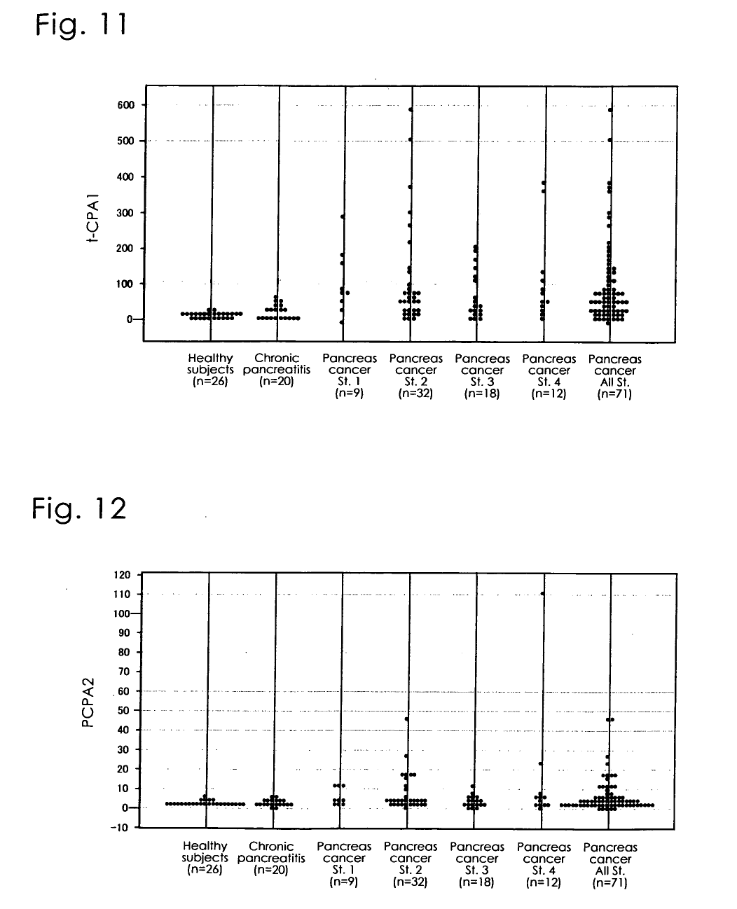

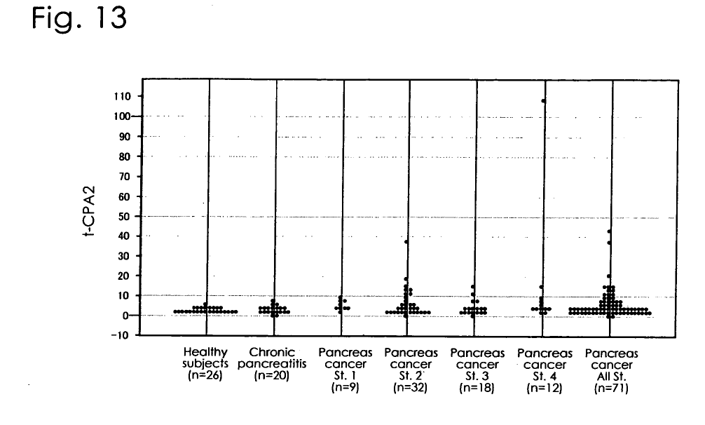

Relationship among an amount of PCPA1 in serum of patients with pancreas cancer, an amount of PCPA1 in serum of patients with chronic pancreatitis, and an amount of PCPA1 in serum of healthy subjects (a distribution diagram of amount of PCPA1 depending on diseases) is shown in FIG. 10. Relationship among an amount of t-CPA1 in serum of patients with pancreas cancer, an amount of t-CPA1 in serum of patients with chronic pancreatitis, and an amount of t-CPA1 in serum of healthy subjects (a distribution diagram of amount of t-CPA1 depending on diseases) is shown in FIG. 11. Relationship among an amount of PCPA2 in serum of patients with pancreas cancer, an amount of PCPA2 in serum of patients with chronic pancreatitis, and an amount of PCPA2 in serum of healthy subjects (a distribution diagram of amount of PCPA2 depending on diseases) is shown in FIG. 12. Relationship among an amount of t-CPA2 in serum of patients with pancreas cancer, an amount of t-CPA2 in serum of patients with chronic pancreatitis, and an amount of t-CPA2 in serum of healthy subjects (a distribution diagram of amount of t-CPA2 depending on diseases) is shown in FIG. 13.

As can be seen from FIG. 7 to FIG. 9, correlation coefficient between PCPA1 and t-CPA1, r=0.9823; the correlation coefficient between PCPA2 and t-CPA2, r=0.9822; and correlation coefficient between PCPA1 and PCPA2, r=0.7034 were derived. Namely, it is suggested that there exists concentration ratio between PCPA1 and PCPA2, and correlation is approved in the concentration ratio.

As apparent from FIG. 10 to FIG. 13, in comparison with levels of PCPA1, t-CPA1, PCPA2 and t-CPA2 in serum of healthy subjects and of patients with chronic pancreatitis, levels of PCPA1, t-CPA1, PCPA2 and t-CPA2 in serum of patients with pancreas cancer (in all stages) exhibit higher. Moreover, since such levels in patients with stage 1 and stage 2 pancreas cancer, that is early stage cancer, exhibit significantly high level, it can be understood that assaying PCPA1, t-CPA1, PCPA2 or t-CPA2 in serum can provide judgment between pancreas cancer, especially early stage pancreas cancer and pancreatitis and normal condition.

When standard value is set as mean+2SD of amount of PCPA1 obtained by using serum of healthy subjects (34 ng/ml), sensitivity for total pancreas cancer is 66%; sensitivity for stage 1 pancreas cancer is 78%; sensitivity for stage 2 pancreas cancer: 66%; sensitivity for stage 3 pancreas cancer is 50%; and sensitivity for stage 4 pancreas cancer is 83%. Specificity for healthy subjects is 96% and specificity for chronic pancreatitis is 75%.

When standard value is set as mean+2SD of amount of t-CPA1 obtained by using serum of healthy subjects (21 ng/ml), sensitivity for total pancreas cancer is 79%; sensitivity for stage 1 pancreas cancer is 89%; sensitivity for stage 2 pancreas cancer is 78%; sensitivity for stage 3 pancreas cancer is 72%; and sensitivity for stage 4 pancreas cancer is 83%. Specificity for healthy subjects is 96% and specificity for chronic pancreatitis is 50%.

When standard value is set as mean+2SD of amount of PCPA2 obtained by using serum of healthy subjects (4.7 ng/ml) is set as a standard value, sensitivity for total pancreas cancer is 38%; a sensitivity for stage 1 pancreas cancer is 44%; sensitivity for stage 2 pancreas cancer is 34%; sensitivity for stage 3 pancreas cancer is 33%; and sensitivity for stage 4 pancreas cancer is 50%. Specificity for healthy subjects is 92% and specificity for chronic pancreatitis is 90%.

When a mean+2SD of amount of t-CPA2 obtained by using serum of the healthy subjects (4.8 ng/ml) is set as a standard value, a sensitivity for total pancreas cancer: 35%; a sensitivity for stage 1 pancreas cancer: 44%; a sensitivity for stage 2 pancreas cancer: 38%; a sensitivity for stage 3 pancreas cancer: 22%; and a sensitivity for stage 4 pancreas cancer: 42%. Specificity for healthy subjects: 96% and specificity for chronic pancreatitis: 90%.

As can be seen from the above, when known immunoassay such as ELISA, EIA, and the like was conducted by using a t-CPA1 specific antibody, a PCPA1 specific antibody, a t-CPA2 specific antibody and a PCPA2 specific antibody of the present invention, it can be understood that t-CPA1, PCPA1, t-CPA2 or PCPA2 in samples can be assayed specifically and simply.

It is suggested to be useful that an amount of t-CPA1, an amount of PCPA1, an amount of t-CPA2 or an amount of PCPA2 in samples obtained by immunoassay using a t-CPA1 specific antibody, a PCPA1 specific antibody, a t-CPA2 specific antibody and a PCPA2 specific antibody of the present invention in samples (i.e. an amount of t-CPA1 in serum assayed using the t-CPA1 specific monoclonal antibody CPA1-7-5 and the t-CPA1 specific monoclonal antibody CPA2-14-76; an amount of PCPA1 in serum assayed using the t-CPA1 specific monoclonal antibody CPA1-7-5 and the PCPA1 specific monoclonal antibody CPA2-15-95; an amount of t-CPA2 in serum assayed using the t-CPA2 specific monoclonal antibody CPA1-1-14 and the t-CPA2 specific monoclonal antibody CPA2-25-118; an amount of PCPA2 in serum assayed using the t-CPA2 specific monoclonal antibody CPA1-1-14 and the PCPA2 specific monoclonal antibody CPA2-11-50) is useful as a judgment (diagnosis) marker of pancreas cancer, especially early stage pancreas cancer.

Effect of the Invention:

The present invention provides an antibody which specifically binds to CPA1 and PCPA1, an antibody which specifically binds to CPA2 and PCPA2, an antibody which specifically binds to PCPA1, an antibody which specifically binds to PCPA2 and a hybridoma which produces a monoclonal antibody thereof; a method for immunologically assaying an amount of at least one kind selected from an amount of CPA1, an amount of CPA2, an amount of PCPA1, an amount of PCPA2 and a total amount of at least two kinds selected therefrom by using at least one kind selected from these antibodies; and a judging method for pancreas cancer. By conducting an immunological assay method employing at least one antibody selected from said antibodies, at least one kind of an amount (an amount of an assay object to be assayed) selected from an amount of CPA1, an amount of CPA2, an amount of PCPA1, an amount of PCPA2 and a total amount of at least two kinds selected therefrom [a total amount of CPA1 and PCPA1 (t-CPA1 amount), a total amount of CPA2 and PCPA2 (t-CPA2 amount), a total amount of CPA1, CPA2, PCPA1 and PCPA2 (total t-CPA amount), a total amount of PCPA1 and PCPA2 (total PCPA amount), a total amount of CPA1 and CPA2 (total CPA amount),] in a sample derived from a living body, can be more simply, more quickly and more specifically assayed than in a conventional method.

And by assaying an amount of an object to be assayed as described above, in a sample derived from a living body, by employing an assay method according to the present invention, and based on the assay results, it is possible to judge pancreas cancer or not, especially pancreas cancer at an early stage (stage 1, 2, and the like) or not.

1. An antibody which specifically binds to carboxypeptidase A1 and procarboxypeptidase A1.

2. An antibody which specifically binds to carboxypeptidase A2 and procarboxypeptidase A2.

3. An antibody which specifically binds to procarboxypeptidase A1.

4. An antibody which specifically binds to procarboxypeptidase A2.

5. The antibody according to anyone of claim 1, wherein the antibody is a monoclonal antibody.

6. A hybridoma which produces the monoclonal antibody according to claim 5.

7. A method for immunologically assaying one or more amount selected from an amount of carboxypeptidase A1, an amount of carboxypeptidase A2, an amount of procarboxypeptidase A1, an amount of procarboxypeptidase A2 and a total amount of two or more amounts selected therefrom which comprises;
using one or more antibody selected from an antibody which specifically binds to carboxypeptidase A1 and procarboxypeptidase A1, an antibody which specifically binds to carboxypeptidase A2 and procarboxypeptidase A2, an antibody which specifically binds to procarboxypeptidase A1 and an antibody which specifically binds to procarboxypeptidase A2.

8. The method according to claim 7, wherein the amount is a total amount of carboxypeptidase A1 and procarboxypeptidase A1 (t-CPA1), and the antibody is an antibody which specifically binds to carboxypeptidase A1 and procarboxypeptidase A1.

9. The method according to claim 7, wherein the amount is a total amount of carboxypeptidase A2 and procarboxypeptidase A2 (t-CPA2), and the antibody is an antibody which specifically binds to carboxypeptidase A2 and procarboxypeptidase A2.

10. The method according to claim 7, wherein the amount is an amount of procarboxypeptidase A1, and the antibody is an antibody which specifically binds to procarboxypeptidase A1.

11. The method according to claim 7, wherein the amount is an amount of procarboxypeptidase A2, and the antibody is an antibody which specifically binds to procarboxypeptidase A2.

12. The method according to claim 7, wherein the amount is a total amount of carboxypeptidase A1, carboxypeptidase A2, procarboxypeptidase A1 and procarboxypeptidase A2 (total t-CPA), and the antibody is an antibody which specifically binds to carboxypeptidase A1 and procarboxypeptidase A2 and an antibody which specifically binds to carboxypeptidase A2 and procarboxypeptidase A2.

13. The method according to claim 7, wherein the amount is a total amount of procarboxypeptidase A1 and procarboxypeptidase A2 (total PCPA), and the antibody is an antibody which specifically binds to procarboxypeptidase A1 and an antibody which specifically binds to procarboxypeptidase A2.

14. The method according to claim 7, wherein the amount is an amount of procarboxypeptidase A1 (CPA 1), and the antibody is an antibody which specifically binds to carboxypeptidase A1 and procarboxypeptidase A1 and an antibody which specifically binds to procarboxypeptidase A1.

15. The method according to claim 7, wherein the amount is an amount of procarboxypeptidase A2 (CPA2), and the antibody is an antibody which specifically binds to carboxypeptidase A2 and procarboxypeptidase A2 and an antibody which specifically binds to procarboxypeptidase A2.

16. The method according to claim 7, wherein the amount is a total amount of carboxypeptidase A1 and carboxypeptidase A2 (total CPA), and the antibody is an antibody which specifically binds to carboxypeptidase A1 and procarboxypeptidase A1, an antibody which specifically binds to carboxypeptidase A2 and procarboxypeptidase A2, an antibody which specifically binds to procarboxypeptidase A1 and an antibody which specifically binds to procarboxypeptidase A2.

17. The method according to claim 7, wherein the antibody is a monoclonal antibody.

18. The method according to claim 7, further using in combination of an antibody which binds to at least one selected from carboxypeptidase A1, carboxypeptidase A2, procarboxypeptidase A1 and procarboxypeptidase A2.

19. The method according to claim 18, wherein the antibody is a monoclonal antibody.

20. The method according to claim 18, wherein either of the antibodies is labeled with a labeling substance.

21. The method according to claim 18, wherein one of the antibodies is immobilized at an insoluble carrier, and the other is labeled with a labeling substance.

22. The method according to claim 21, wherein the method is performed by
forming one or more complexs selected from (i) a complex of an antibody immobilized on an insoluble carrier, carboxypeptidase A1 in the sample and an antibody labeled by a labeling substance, (ii) a complex of an antibody immobilized on an insoluble carrier, carboxypeptidase A2 in the sample and an antibody labeled by a labeling substance, (iii) a complex of an antibody immobilized on an insoluble carrier, procarboxypeptidase A1 in the sample and an antibody labeled by a labeling substance and (iv) a complex of an antibody immobilized on an insoluble carrier, procarboxypeptidase A2 in the sample and an antibody labeled by a labeling substance, and
assaying one or more amounts selected from an amount of carboxypeptidase A1, an amount of carboxypeptidase A2, an amount of procarboxypeptidase A1, procarboxypeptidase A2 and a total amount of two or more selected therefrom in the sample on the basis of an amount of the labeling substance in said complex.

23. The method according to claim 21, wherein the method is performed by
(i) contacting an antibody immobilized on an insoluble carrier with an sample to form one or more complex (complex-i) selected from (a) a complex of said immobilized antibody and carboxypeptidase A1 in the sample, (b) a complex of said immobilized antibody and carboxypeptidase A2 in the sample, (c) a complex of said immobilized antibody and procarboxypeptidase A1 in the sample and (d) a complex of said immobilized antibody and procarboxypeptidase A2 in the sample,
(ii) contacting the complex-i with an antibody labeled by a labeling substance to form a complex (complex-ii) of said complex-i and said labeled antibody, and
(iii) assaying one or more amounts selected from an amount of carboxypeptidase A1, an amount of carboxypeptidase A2, an amount of procarboxypeptidase A1, procarboxypeptidase A2 and a total amount of two or more selected therefrom in the sample on the basis of an amount of the labeling substance in said complex-ii.

24. A method for judgment of pancreas cancer which comprises;
assaying one or more amounts selected from an amount of carboxypeptidase A1, an amount of carboxypeptidase A2, an amount of procarboxypeptidase A1 and procarboxypeptidase A2 in a sample, and
judging whether the assay result indicates pancreas cancer or not“on the basis of thus obtained value.

25. The method according to claim 24, wherein the one or more amounts selected from an amount of carboxypeptidase A1, an amount of carboxypeptidase A2, an amount of procarboxypeptidase A1 and procarboxypeptidase A2 in a sample is assayed.

26. The method according to claim 24, wherein the judgment is performed on the basis of a difference obtained by comparing (1) the one or more amounts selected from an amount of carboxypeptidase A1, an amount of carboxypeptidase A2, an amount of procarboxypeptidase A1 and procarboxypeptidase A2 in a sample with (2) a control.

27. The method according to claim 26, wherein one or more amount selected from an amount of carboxypeptidase A1, an amount of carboxypeptidase A2, an amount of procarboxypeptidase A1 and an amount of procarboxypeptidase A2 in a sample is assayed.

28. The method according to claim 24, wherein the method is performed by
assaying (1) one or more amounts selected from an amount of carboxypeptidase A1, an amount of carboxypeptidase A2, an amount of procarboxypeptidase A1 and procarboxypeptidase A2 in the sample, and (2) one or more amounts selected from (a) a total amount of carboxypeptidase A1 and procarboxypeptidase A1 (t-CPA1), (b) a total amount of carboxypeptidase A2 and procarboxypeptidase A2 (t-CPA2), (c) a total amount of carboxypeptidase A1 and carboxypeptidase A2 (total CPA), (d) a total amount of procarboxypeptidase A1 and procarboxypeptidase A2 (total PCPA) and (e) a total amount of carboxypeptidase A1, carboxypeptidase A2, procarboxypeptidase A1 and procarboxypeptidase A2 (total t-CPA) in the sample, and
judging whether the assay result indicates pancreas cancer or not” on the basis of a ratio between said amount obtained by (1) and said total amount obtained by (2).

29. The method according to claim 28, wherein the one or more amount selected from an amount of carboxypeptidase A1, an amount of carboxypeptidase A2, an amount of procarboxypeptidase A1 and an amount of procarboxypeptidase A2 in a sample, and/or the one or more amounts selected from said (a) to (e) is assayed.

30. The method according to claim 24, wherein the one or more amount selected from an amount of carboxypeptidase A1, an amount of carboxypeptidase A2, an amount of procarboxypeptidase A1 and an amount of procarboxypeptidase A2 in a sample, is an amount of carboxypeptidase A1, an amount of carboxypeptidase A2, an amount of procarboxypeptidase A1, an amount of procarboxypeptidase A2, a total amount (t-CPA 1) of carboxypeptidase A1 and procarboxypeptidase A1, or a total amount (t-CPA2) of carboxypeptidase A2 and procarboxypeptidase A2 in a sample.

31. A kit for assaying one or more selected from an amount of carboxypeptidase A1, an amount of carboxypeptidase A2, an amount of procarboxypeptidase A1, an amount of procarboxypeptidase A2 and a total amount of two or more amounts selected therefrom comprising one or more antibody according to claim 1.

32. The kit according to claim 31, wherein the antibody is a monoclonal antibody.

33. The kit according to claim 31, further comprising an antibody which binds to one or more selected from carboxypeptidase A1, carboxypeptidase A2, procarboxypeptidase A1 and procarboxypeptidase A2.

34. The kit according to claim 33, wherein the antibody is a monoclonal antibody.

35. The kit according to claim 33, wherein either of the antibodies is labeled with a labeling substance.

36. The kit according to claim 33, wherein one of the antibodies is immobilized at an insoluble carrier and the other is labeled with a labeling substance.

37. A kit for judging of pancreas cancer which comprises one or more antibody according to claim 1.


SEQUENCE LISTING (14 sequences)

SEQ ID NO: 1 - DNA, 30 bp, Artificial
  Oligonucleotide Primer
atgcgggggttgctggtgttgagtgtcctg

SEQ ID NO: 2 - DNA, 30 bp, Artificial
  Oligonucleotide Primer
tcagtaggggtgattcagggtgtgctccat

SEQ ID NO: 3 - DNA, 57 bp, Artificial
  Oligonucleotide Primer
ccgaattcacatcatcaccatcaccataaggaggactttgtggggcatcaggtgctc

SEQ ID NO: 4 - DNA, 40 bp, Artificial
  Oligonucleotide Primer
ccgaattcgcgtaggggtgattcagggtgtgctccatgat

SEQ ID NO: 5 - DNA, 1260 bp, HUMAN
  CDS (1)..(1257)
atgcgggggttgctggtgttgagtgtcctgttgggggctgtctttggcaaggaggactttgtggggcatcaggtgctccgaatctctgtagccgatgaggcccaggtacagaaggtgaaggagctggaggacctggagcacctgcagctggacttctggcgggggcctgcccaccctggctcccccatcgacgtccgagtgcccttccccagcatccaggcggtcaagatctttctggagtcccacggcatcagctatgagaccatgatcgaggacgtgcagtcgctgctggacgaggagcaggagcagatgttcgccttccggtcccgggcgcgctccaccgacacttttaactacgccacctaccacaccctggaggagatctatgacttcctggacctgctggtggcggagaacccgcaccttgtcagcaagatccagattggcaacacctatgaagggcgtcccatttatgtgctgaagttcagcacggggggcagtaagcgtccagccatctggatcgacacgggcatccattcccgggagtgggtcacccaggccagtggggtctggtttgcaaagaagatcactcaagactatgggcaggatgcagctttcaccgccattctcgacaccttggacatcttcctggagatcgtcaccaaccctgatggctttgccttcacgcacagcacgaatcgcatgtggcgcaagactcggtcccacacagcaggctccctctgtattggcgtggaccccaacaggaactgggacgctggctttgggttgtccggagccagcagtaacccctgctcggagacttaccacggcaagtttgccaattccgaagtggaggtcaagtccattgtagactttgtgaaggaccatgggaacatcaaggccttcatctccatccacagctactcccagctcctcatgtatccctatggctacaaaacagaaccagtccctgaccaggatgagctggatcagctttccaaggctgctgtgacagccctggcctctctctacgggaccaagttcaactatggcagcatcatcaaggcaatttatcaagccagtggaagcactattgactggacctacagccagggcatcaagtactccttcaccttcgagctccgggacactgggcgctatggcttcctgctgccagcctcccagatcatccccacagccaaggagacgtggctggcgcttctgaccatcatggagcacaccctgaatcacccctactga

SEQ ID NO: 6 - protein, 419 aa, HUMAN
MRGLLVLSVLLGAVFGKEDFVGHQVLRISVADEAQVQKVKELEDLEHLQLDFWRGPAHPGSPIDVRVPFPSIQAVKIFLESHGISYETMIEDVQSLLDEEQEQMFAFRSRARSTDTFNYATYHTLEEIYDFLDLLVAENPHLVSKIQIGNTYEGRPIYVLKFSTGGSKRPAIWIDTGIHSREWVTQASGVWFAKKITQDYGQDAAFTAILDTLDIFLEIVTNPDGFAFTHSTNRMWRKTRSHTAGSLCIGVDPNRNWDAGFGLSGASSNPCSETYHGKFANSEVEVKSIVDFVKDHGNIKAFISIHSYSQLLMYPYGYKTEPVPDQDELDQLSKAAVTALASLYGTKFNYGSIIKAIYQASGSTIDWTYSQGIKYSFTFELRDTGRYGFLLPASQIIPTAKETWLALLTIMEHTLNHPY

SEQ ID NO: 7 - DNA, 30 bp, Artificial
  Oligonucleotide Primer
atgaggttgatcctgttttttggtgccctt

SEQ ID NO: 8 - DNA, 30 bp, Artificial
  Oligonucleotide Primer
ctaataggggtggtctcgcacatgctccat

SEQ ID NO: 9 - DNA, 57 bp, Artificial
  Oligonucleotide Primer
ccgaattcacatcatcaccatcaccatctagaaacatttgtgggagaccaagttctt

SEQ ID NO: 10 - DNA, 40 bp, Artificial
  Oligonucleotide Primer
ccgaattcgcataggggtggtctcgcacatgctccatgat

SEQ ID NO: 11 - DNA, 1254 bp, HUMAN
  CDS (1)..(1251)
atgaggttgatcctgttttttggtgccctttttgggcatatctactgtctagaaacatttgtgggagaccaagttcttgagattgtaccaagcaatgaagaacaaattaaaaatctgctacaattggaggctcaagaacatctccagcttgatttttggaaatcacccaccaccccaggggagacagcccacgtccgagttcccttcgtcaacgtccaggcagtcaaagtgttcttggggtcccagggaattgcctattccatcatgattgaagacgtgcaggtcctgttggacaaagagaatgaagaaatgctttttaataggagaagagaacggagtggtaacttcaattttggggcctaccataccctggaagagatttcccaagaaatggataacctcgtggctgagcaccctggtctagtgagcaaagtgaatattggctcttcttttgagaaccggcctatgaacgtgctcaagttcagcaccggaggagacaagccagctatctggctggatgctgggatccatgctcgagagtgggttacacaagctacggcactttggacagcaaataagattgtttctgattatggaaaggacccatccatcacttccattctggacgccctggatatcttcctcctgccagtcacaaaccctgatggatacgtgttctctcaaaccaaaaatcgtatgtggcggaagacccggtccaaggtatctggaagcctctgtgttggtgtggatcctaaccggaactgggatgcaggttttggaggacctggagccagcagcaacccttgctctgattcataccacggacccagtgccaactctgaagttgaagtgaaatccatagtggacttcatcaagagtcatggaaaagtcaaggccttcattaccctccacagctattcccagctgctgatgttcccctatgggtacaaatgtaccaagttagatgactttgatgagctgagtgaagtggcccaaaaggctgcccaatctctgagaagcctgcatggcaccaagtacaaagtgggaccaatctgctctgtcatctaccaagccagtggaggaagcattgactggtcctatgattatggcatcaagtactcatttgcctttgaactgagagacacagggcgctacggcttcctcttgccagcccgtcagatcctgcccacagccgaggagacctggcttggcttgaaggcaatcatggagcatgtgcgagaccacccctattag

SEQ ID NO: 12 - protein, 417 aa, HUMAN
MRLILFFGALFGHIYCLETFVGDQVLEIVPSNEEQIKNLLQLEAQEHLQLDFWKSPTTPGETAHVRVPFVNVQAVKVFLGSQGIAYSIMIEDVQVLLDKENEEMLFNRRRERSGNFNFGAYHTLEEISQEMDNLVAEHPGLVSKVNIGSSFENRPMNVLKFSTGGDKPAIWLDAGIHAREWVTQATALWTANKIVSDYGKDPSITSILDALDIFLLPVTNPDGYVFSQTKNRMWRKTRSKVSGSLCVGVDPNRNWDAGFGGPGASSNPCSDSYHGPSANSEVEVKSIVDFIKSHGKVKAFITLHSYSQLLMFPYGYKCTKLDDFDELSEVAQKAAQSLRSLHGTKYKVGPICSVIYQASGGSIDWSYDYGIKYSFAFELRDTGRYGFLLPARQILPTAEETWLGLKAIMEHVRDHPY

SEQ ID NO: 13 - DNA, 41 bp, Artificial
  Oligonucleotide Primer
ccgaattctcaccgggaccggaaggcgaacatctgctcctg

SEQ ID NO: 14 - DNA, 41 bp, Artificial
  Oligonucleotide Primer
ccgaattctcacctattaaaaagcatttcttcattctcttt